(19) United States
(12) Patent Application Publication
(10) Pub. No.: US 2024/0180485 A1
(43) Pub. Date: Jun. 6, 2024

(54) METHOD AND DEVICE FOR INDUCING HYPOMETABOLIC STATE IN SUBJECT WITH DISEASE

(71) Applicants: UNIVERSITY OF TSUKUBA, Ibaraki (JP); RIKEN, Saitama (JP)

(72) Inventors: Takeshi SAKURAI, Ibaraki (JP); Genshiro SUNAGAWA, Saitama (JP)

(73) Assignees: University of Tsukuba, Ibaraki (JP); RIKEN, Saitama (JP)

(21) Appl. No.: 18/553,441

(22) PCT Filed: Mar. 30, 2022
(86) PCT No.: PCT/JP2022/015819

§ 371 Date: Sep. 29, 2023

(30) Foreign Application Priority Data
Mar. 31, 2021 (JP) 2021-059600

(51) Int. Cl. A61B 5/00; A61B 5/01; A61B 5/083; A61N 1/05; A61N 1/36

(52) U.S. Cl. CPC A61B 5/4836; A61B 5/01; A61B 5/0833; A61B 5/7275; A61B 5/7282; A61B 5/7405; A61B 5/742; A61B 5/746; A61N 1/0534; A61N 1/36121; A61N 1/36135; A61B 2560/0242; A61B 2562/0271

(57) ABSTRACT

The present invention provides a method for inducing a hibernation-like state and an apparatus therefor. According to the present invention, provided is a method for decreasing a theoretical setpoint temperature of body temperature and a feedback gain of heat generation in a subject or a method for inducing a hibernation-like state in a subject, the method being a chemical and/or physical method including providing an excitatory stimulus to a pyroglutamylated RFamide peptide (QRFP)-producing neuron. According to the present invention, an apparatus used to practice this method is also provided.

### TECHNICAL FIELD

The present invention provides a method for inducing a hypometabolic state in a subject having a disease, and an apparatus therefor.

### BACKGROUND ART

Inducing a hypometabolic state (for example, hibernation-like state) in a subject having an acute serious disease can slow progression of the disease or prolong the time until death of the subject from the disease. Separately from this, hypothermic therapy has been developed for a disease. For example, the effectiveness of hypothermic therapy for neonatal hypoxic ischemic encephalopathy has been disclosed⁴¹. Currently, this is the only hypothermic therapy that has been proven effective. In contrast, a study has shown that hypothermic therapy after cardiac arrest does not improve prognosis compared with targeted temperature management (maintaining 36° C.)⁴². Because of this study, hypothermic therapy in an adult has been less actively carried out.

Examples of the hypometabolic state include a hibernation-like state. A homoiothermic animal, a bird, and a mammal consume most of their body energy for heat production in order to maintain the internal body temperature (T_{B}) within a narrow range higher than the ambient temperature (T_{A}). However, some mammals actively decrease their metabolism and enter a state known as hibernation in order to survive a winter food shortage. The animals return to a normal state without obvious tissue damage^{1,2}. A mouse does not hibernate, but exhibits a short-term hypometabolic state known as daily torpor when the mouse can benefit from a decrease in basal metabolism. The reduction in energy consumption in both hibernation and daily torpor is mainly achieved by a decrease in metabolism, which is influenced by two major factors, that is, the theoretical setpoint temperature (TR) and the negative feedback gain (H) of heat generation. In a mouse undergoing daily torpor, TR remains close to normal, but H decreases to nearly one-tenth of normal, resulting in a TB much lower than TR³. In contrast, in hibernation, both TR and H significantly decrease, allowing the maintenance of a hypometabolic state that is more efficient than daily torpor and can respond to a fluctuation in outside air temperature^{4,5}. It has been established by many experiments that such active hypometabolism is regulated by the central nervous system⁶. However, the neural mechanism remains entirely unclear. Clarification of the mechanisms of daily torpor and/or hibernation is a step that is necessary for developing a method for artificially inducing an artificial hibernation-like hypometabolic state in a non-hibernating animal including a human^{1,7}; further, the clarification will also be beneficial in long-range space exploration in the future. Here, it has been found that excitatory manipulation of a novel chemically defined neuron population in the hypothalamus causes a hypometabolic/hypothermic state over a very long period of time in a mouse. In this state, the metabolic rate decreases to one-third or less, but unlike in an anesthetized state, the mouse still reacts to a change in ambient temperature. Further, the mouse recovered naturally from this state without an obvious abnormality. This discovery is an important finding for the hibernation mechanism and the development of a method for inducing an artificial hibernation-like state. Artificial induction of a hibernation-like state in a rodent such as a mouse and a rat has recently been reported⁴³.

### SUMMARY OF INVENTION

The present invention provides a method for inducing a hypometabolic state (preferably a hibernation-like state) in a subject having a disease and an apparatus therefor.

The present inventors have found that by applying an excitatory stimulus to a pyroglutamylated RFamide peptide (QRFP) gene-expressing neuron in a region including the anteroventral periventricular nucleus (AVPe), the medial preoptic area (MPA), and the periventricular nucleus (Pe) of the hypothalamus in the brain of a living, non-hibernating animal, a hibernation-like state can be induced in the subject. The present inventors have also found that when a subject has a disease, induction of a hibernation-like state that can decrease overall vital activity delays the progression and exacerbation of the disease and improves the survival rate of the subject. The inventors have also found a method for distinguishing between a hypometabolic state (preferably a hibernation-like state) and death by oxygen consumption.

According to the present invention, for example, the following inventions are provided.

(1) An apparatus that monitors a hypometabolic state in a subject having a disease in a controlled hypometabolic state (preferably a hibernation-like state), the apparatus comprising:
a measurement unit that measures an oxygen concentration of each exhaled air and inhaled air from the subject;
a computation unit that calculates an oxygen consumption of the subject from an oxygen concentration difference between the exhaled air and the inhaled air from the subject; and
an oxygen monitoring unit that monitors the oxygen consumption of the subject over time; and optionally further comprising:
a display unit that shows a result of oxygen monitoring, or optionally further comprising:
a hypometabolic state monitoring unit that estimates that the subject is in a hypometabolic state (preferably a hibernation-like state) when the oxygen consumption decreases compared with before the hypometabolic state, and estimates that the subject is dead or may be dead when the oxygen consumption is zero.
   (1′) An apparatus that, in a subject having a disease in a controlled hypometabolic state (preferably a hibernation-like state), monitors the hypometabolic state (preferably a hibernation-like state), the apparatus comprising:
a measurement unit that measures an oxygen concentration of each exhaled air and inhaled air from the subject;
a computation unit that calculates an oxygen consumption of the subject from an oxygen concentration difference between the exhaled air and the inhaled air from the subject;
a hypometabolic state (preferably a hibernation-like state) possibility estimation unit that estimates that the subject has entered a hypometabolic state (preferably a hibernation-like state) or may have entered a hypometabolic state (preferably a hibernation-like state) at least based on a decrease in oxygen consumption (and preferably theoretical setpoint temperature) of the subject compared with an oxygen consumption (and preferably theoretical setpoint temperature) of the subject before entering the hypometabolic state (preferably a hibernation-like state); and
a death possibility estimation unit that estimates that the subject is dead or may be dead when the oxygen consumption is zero.
   (1″) An apparatus that, in a subject having a disease in a hibernation-like state, monitors the hibernation-like state, the apparatus comprising:
a measurement unit that measures an oxygen concentration of each exhaled air and inhaled air from the subject;
a computation unit that calculates an oxygen consumption of the subject from an oxygen concentration difference between the exhaled air and the inhaled air from the subject;
a hibernation-like state possibility estimation unit that estimates that the subject has entered a hibernation-like state or may have entered a hibernation-like state at least based on a decrease in oxygen consumption (and preferably theoretical setpoint temperature) of the subject compared with an oxygen consumption (and preferably theoretical setpoint temperature) of the subject before entering the hibernation-like state; and a death possibility estimation unit that estimates that the subject is dead or may be dead when the oxygen consumption is zero.
   (1′″) An apparatus that, in a subject having a disease in a hibernation-like state, monitors the hibernation-like state, the apparatus comprising:
a measurement unit that measures an oxygen concentration of each exhaled air and inhaled air from the subject;
a computation unit that calculates an oxygen consumption of the subject from an oxygen concentration difference between the exhaled air and the inhaled air from the subject;
a hibernation-like state possibility estimation unit that estimates that the subject has entered a hibernation-like state or may have entered a hibernation-like state at least based on a decrease in both oxygen consumption and theoretical setpoint temperature of the subject compared with an oxygen consumption and a theoretical setpoint temperature of the subject before entering the hibernation-like state; and
a death possibility estimation unit that estimates that the subject is dead or may be dead when the oxygen consumption is zero
{preferably, optionally further comprising an output device (preferably a display device, preferably a display or a speaker) that outputs an estimation result obtained by each of the estimation units}.
   (2) The apparatus according to (1) above, wherein the controlled hypometabolic state is a hibernation-like state.
   (3) The apparatus according to (1) or (2) above, wherein the apparatus further comprises a determination unit that records a fluctuation in oxygen consumption for a certain period of time in the subject in a stable hypometabolism state, sets a specified range based on the fluctuation in oxygen consumption for a certain period of time, and determines whether the oxygen consumption is within or outside the specified range.
   (4) The apparatus according to any one of (1) to (3) above, wherein the apparatus further comprises an output device, and the apparatus, when the oxygen consumption exceeds an upper limit of the specified range, outputs to the output device an indication that the subject may be waking up from the controlled hypometabolic state; when the oxygen consumption decreases below a lower limit of the specified range, outputs to the output device (preferably a display device, preferably a display) an indication that the subject may be transitioning from the controlled hypometabolic state to greater hypometabolism; and when the oxygen consumption is within the specified range, outputs to the output device (preferably a display device, preferably a display or a speaker) an indication that the subject is in the controlled hypometabolism state.
   (5) The apparatus according to any one of (1′″) and (2) to (4) above, wherein the apparatus further comprises a deep body thermometer, a body temperature storage unit that stores a body temperature measured by the thermometer, a computation unit that calculates the theoretical setpoint temperature, and a computation unit that calculates a feedback gain (H) of heat generation.
   (6) An apparatus that stimulates a pyroglutamylated RFamide peptide (QRFP)-producing neuron within one or more regions selected from the group consisting of an anteroventral periventricular nucleus (AVPe), a medial preoptic area (MPA), and a periventricular nucleus (Pe) in a brain of a living subject, wherein the subject preferably has a disease, and
the apparatus comprises:
a control unit that transmits a control signal controlling voltage generation;
a voltage generation unit that receives the control signal from the control unit and generates a voltage;
a stimulation probe electrically connected proximally to the voltage generation unit and comprising an electrical stimulation electrode distally wherein the stimulation probe has a length sufficient to access the QRFP-producing neuron from a brain surface, and generates an electrical stimulus at a distal electrical stimulation electrode with the voltage from the voltage generation unit;
an outside air temperature gauge;
a deep body thermometer;
an exhaled gas analysis unit that measures an oxygen concentration of exhaled gas;
a recording unit that records a measured outside air temperature and at least one numerical value selected from the group consisting of a deep body temperature and the oxygen concentration;
a computation unit that calculates an oxygen consumption of the subject from an oxygen concentration difference between exhaled air and inhaled air from the subject;
a hibernation possibility determination unit that determines that the subject has entered a hypometabolic state (preferably a hibernation-like state) or may have entered a hypometabolic state (preferably a hibernation-like state) at least based on a decrease in both oxygen consumption and theoretical setpoint temperature compared with an oxygen consumption and a theoretical setpoint temperature of the subject before entering the hypometabolic state (preferably a hibernation-like state); and
a death possibility determination unit that determines that the subject is dead or may be dead at least based on an oxygen consumption of zero.
   (7) An apparatus that stimulates a pyroglutamylated RFamide peptide (QRFP)-producing neuron within one or more regions selected from the group consisting of an anteroventral periventricular nucleus (AVPe), a medial preoptic area (MPA), and a periventricular nucleus (Pe) in a brain of a living subject, wherein the subject preferably has a disease, and
the apparatus comprises:
a control unit that transmits a control signal controlling release of a QRFP-producing neuron stimulating compound;
a storage unit for the compound;
a compound export unit that receives the control signal from the control unit and exports the compound from the storage unit for the compound;
a guide comprising a compound release port and a channel for the compound to the release port and delivering the compound to the QRFP-producing neuron;
an outside air temperature gauge;
a deep body thermometer;
an exhaled gas analysis unit that measures an oxygen concentration of exhaled gas;
a recording unit that records a measured outside air temperature and at least one numerical value selected from the group consisting of a deep body temperature and the oxygen concentration;
a computation unit that calculates an oxygen consumption of the subject from an oxygen concentration difference between exhaled air and inhaled air from the subject;
a hibernation possibility estimation unit that estimates that the subject has entered a hibernation-like state or may has entered a hibernation-like state at least based on a decrease in both oxygen consumption and theoretical setpoint temperature compared with an oxygen consumption and a theoretical setpoint temperature of the subject in a non-hibernation-like state; and
a death possibility estimation unit that estimates that the subject is dead or may be dead at least based on an oxygen consumption of zero.
   (8) The apparatus according to (6) or (7) above, wherein the apparatus further comprises a determination unit that determines whether the subject is in a hypothermic state from the outside air temperature and the deep body temperature recorded in the recording unit.
   (9) The apparatus according to any one of (6) to (8) above, wherein the apparatus further comprises a determination unit that determines whether or not the subject is in a hypometabolic state from the outside air temperature, the deep body temperature, and the oxygen concentration recorded in the recording unit.
   (10) The apparatus according to any one of (6) to (9) above, wherein the hibernation possibility determination unit further comprises a determination unit that determines whether or not the subject is in a hibernation-like state based on the outside air temperature, the deep body temperature, and the oxygen concentration recorded in the recording unit.
   (11) The apparatus according to any one of (8) to (10) above, wherein the control unit transmits a control signal for stimulating the QRFP-producing neuron continuously or intermittently until it is determined that the subject is in any one state selected from the group consisting of a hypothermic state, a hypometabolic state, and a hibernation-like state.
   (12) A method for decreasing a theoretical setpoint temperature of a body temperature in a mammalian subject, the method comprising providing an excitatory stimulus to a pyroglutamylated RFamide peptide (QRFP)-producing neuron, wherein the mammal suffers from a disease.
   (13) The method according to (12) above, wherein the QRFP-producing neuron is a neuron in one or more regions selected from the group consisting of an anteroventral periventricular nucleus (AVPe), a medial preoptic area (MPA), and a periventricular nucleus (Pe).
   (14) The method according to (12) or (13) above, wherein the excitatory stimulus is a stimulus selected from the group consisting of a chemical stimulus, a magnetic stimulus, and an electrical stimulus.
   (15) A method for screening for a substance that provides an excitatory stimulus to a pyroglutamylated RFamide peptide (QRFP)-producing neuron present within regions of an anteroventral periventricular nucleus (AVPe), a medial preoptic area (MPA), and a periventricular nucleus (Pe) in a mammalian animal, preferably a mammalian animal having a disease, the method comprising:
contacting a test compound with the QRFP-producing neuron;
measuring excitation of the QRFP-producing neuron; and
selecting a test compound that provides an excitatory stimulus to the QRFP-producing neuron.
   (15a) A method for screening for a substance that specifically provides an excitatory stimulus to a pyroglutamylated RFamide peptide (QRFP)-producing neuron present within regions of an anteroventral periventricular nucleus (AVPe), a medial preoptic area (MPA), and a periventricular nucleus (Pe) in a mammalian animal, preferably a mammalian animal having a disease, the method comprising:
causing a cell to express a receptor specifically expressed in the QRFP-producing neuron;
contacting a test compound with the cell;
measuring excitation of the QRFP-producing neuron; and
selecting a test compound that provides an excitatory stimulus to the QRFP-producing neuron.
   (15b) A method for testing a substance, or a composition comprising the substance (for example, pharmaceutical formulation and pharmaceutical composition), that specifically provides an excitatory stimulus to a pyroglutamylated RFamide peptide (QRFP)-producing neuron present in regions of an anteroventral periventricular nucleus (AVPe), a medial preoptic area (MPA), and a periventricular nucleus (Pe) in a mammalian animal, preferably a mammalian animal having a disease, the method comprising:
providing a pyroglutamylated RFamide peptide (QRFP)-producing neuron;
contacting a test compound or the composition with the cell;
measuring excitation of the QRFP-producing neuron; and
determining whether the test compound or the composition provides an excitatory stimulus to the QRFP-producing neuron by comparing the excitation of the QRFP-producing neuron before and after the contact with the test compound.
   (15c) A method for testing a substance, or a composition comprising the substance (for example, pharmaceutical formulation and pharmaceutical composition), that specifically provides an excitatory stimulus to a pyroglutamylated RFamide peptide (QRFP)-producing neuron present within regions of an anteroventral periventricular nucleus (AVPe), a medial preoptic area (MPA), and a periventricular nucleus (Pe) in a mammalian animal, preferably a mammalian animal having a disease, the method comprising:
administering a test compound to the regions of the anteroventral periventricular nucleus (AVPe), the medial preoptic area (MPA), and the periventricular nucleus (Pe);
measuring excitation (for example, potential) of the QRFP-producing neuron; and
determining whether the test compound or the composition provides an excitatory stimulus to the QRFP-producing neuron by comparing the excitation of the QRFP-producing neuron before and after contact with the test compound or the composition.
   (15d) A method for testing a test compound, or a composition comprising the compound (for example, pharmaceutical formulation and pharmaceutical composition), that induces hibernation in a mammalian animal, preferably a mammalian animal having a disease, the method comprising:
administering the test compound or the composition to regions of an anteroventral periventricular nucleus (AVPe), a medial preoptic area (MPA), and a periventricular nucleus (Pe) of the mammalian animal (for example, non-human mammalian animal); and
confirming that the mammalian animal hibernates.
   (15e) The method according to (15d) above, wherein
from a correlation between a deep body temperature (for example, intestinal temperature) and an oxygen consumption of the mammalian animal (for example, non-human mammalian animal), the deep body temperature (theoretical setpoint temperature) when the oxygen consumption is assumed to be 0 and ΔVO₂/ΔT_{B} (feedback gain of heat generation) are estimated; and
a decrease in both theoretical setpoint temperature and negative feedback gain of heat generation by the administration of the test compound or the composition comprising the compound (for example, pharmaceutical formulation and pharmaceutical composition) compared with before the administration shows that the mammalian animal has hibernated.
   (15f) A method for testing whether or not a test compound, or a composition comprising the compound (for example, pharmaceutical formulation and pharmaceutical composition), induces hibernation in a mammalian animal such as a human, preferably a mammalian animal having a disease, the method comprising:
providing an estimated value of a theoretical setpoint temperature and an estimated value of a feedback gain of heat generation of a human in whom the test compound or the composition has been administered to regions of an anteroventral periventricular nucleus (AVPe), a medial preoptic area (MPA), and a periventricular nucleus (Pe), and an estimated value of the theoretical setpoint temperature and an estimated value of the feedback gain of heat generation of the human before the administration; and
confirming whether both the estimated value of the theoretical setpoint temperature and the estimated value of the feedback gain of heat generation after the administration decrease compared with the estimated value of the theoretical setpoint temperature and the estimated value of the feedback gain of heat generation before the administration of the test compound or the composition, wherein
a decrease in both the estimated value of the theoretical setpoint temperature and the estimated value of the feedback gain of heat generation after the administration, compared with before the administration, shows that the mammalian animal has hibernated.
   (15g) A method for determining (predicting, estimating, computationally calculating) whether or not a test compound, or a composition comprising the compound (for example, pharmaceutical formulation and pharmaceutical composition), induces hibernation in a mammalian animal such as a human, preferably a mammalian animal having a disease, the method comprising:
providing an estimated value of a theoretical setpoint temperature and an estimated value of a feedback gain of heat generation of a human in whom the test compound or the composition has been administered to regions of an anteroventral periventricular nucleus (AVPe), a medial preoptic area (MPA), and a periventricular nucleus (Pe), and an estimated value of the theoretical setpoint temperature and an estimated value of the feedback gain of heat generation of the human before the administration; and
confirming whether both the estimated value of the theoretical setpoint temperature and the estimated value of the feedback gain of heat generation after the administration decrease compared with the estimated value of the theoretical setpoint temperature and the estimated value of the feedback gain of heat generation before the administration of the test compound or the composition, wherein
a decrease in both the estimated value of the theoretical setpoint temperature and the estimated value of the feedback gain of heat generation after the administration, compared with before the administration, shows that the mammalian animal has hibernated.
   (15h) A method for determining (predicting, estimating, computationally calculating) whether or not a test compound, or a composition comprising the compound (for example, pharmaceutical formulation and pharmaceutical composition), induces hibernation in a mammalian animal such as a human, preferably a mammalian animal having a disease, the method comprising:
recording an oxygen consumption and a deep body temperature under at least two different surrounding environment temperature conditions each of before administration and after administration of the test compound or the composition in a mammalian animal such as a human in whom the test compound or the composition has been administered to regions of an anteroventral periventricular nucleus (AVPe), a medial preoptic area (MPA), and a periventricular nucleus (Pe);
estimating a correlation between the oxygen consumption and the deep body temperature each of before the administration and after the administration; and
determining whether or not an extent of a decrease in oxygen consumption when the deep body temperature decreases after the administration compared with before the administration, and determining whether or not an estimated value of the deep body temperature when the oxygen consumption is assumed to be 0 decreases after the administration compared with before the administration, from the estimated correlation,
wherein a decrease in the extent of a decrease in oxygen consumption when the deep body temperature decreases, after the administration compared with before the administration, and a decrease in the estimated value of the deep body temperature when the oxygen consumption is assumed to be 0, after the administration compared with before the administration, show that the mammalian animal has hibernated.
   (16) A method for determining (testing, predicting, estimating, computationally calculating) whether or not a test compound, or a composition comprising the compound (for example, pharmaceutical formulation and pharmaceutical composition), induces hibernation in a mammalian animal such as a human, preferably a mammalian animal having a disease, the method comprising:
providing (or recording) an oxygen consumption and a deep body temperature recorded under each of at least two different surrounding environment temperature conditions each of before administration and after administration of the test compound or the composition in a mammalian animal such as a human in whom the test compound or the composition has been administered to regions of an anteroventral periventricular nucleus (AVPe), a medial preoptic area (MPA), and a periventricular nucleus (Pe);
estimating a correlation between the oxygen consumption and the deep body temperature each of before the administration and after the administration; and
determining whether or not an extent of a decrease in oxygen consumption when the deep body temperature decreases after the administration compared with before the administration, and determining whether or not an estimated value of the deep body temperature when the oxygen consumption is assumed to be 0 decreases after the administration compared with before the administration, from the estimated correlation,
wherein a decrease in the extent of a decrease in oxygen consumption when the deep body temperature decreases, after the administration compared with before the administration, and a decrease in the estimated value of the deep body temperature when the oxygen consumption is assumed to be 0, after the administration compared with before the administration, show that the mammalian animal has hibernated.
   (17) An apparatus that monitors a hibernating state, the apparatus comprising:
a recording unit that records an oxygen consumption and a deep body temperature recorded under each of at least two different surrounding environment temperature conditions each of before administration and after administration of a test compound, or a composition comprising the compound (for example, pharmaceutical formulation and pharmaceutical composition) in a mammalian animal such as a human, preferably a mammalian animal having a disease, in whom the test compound or the composition has been administered to regions of an anteroventral periventricular nucleus (AVPe), a medial preoptic area (MPA), and a periventricular nucleus (Pe); and
a computation unit that estimates a correlation between the oxygen consumption and the deep body temperature each of before the administration and after the administration, and determines whether or not an extent of a decrease in oxygen consumption when the deep body temperature decreases after the administration compared with before the administration, and determines whether or not an estimated value of the deep body temperature when the oxygen consumption is assumed to be 0 decreases after the administration compared with before the administration, from the estimated correlation; and
comprising:
a determination unit that determines that the mammalian animal has hibernated in the case of a decrease in the extent of a decrease in oxygen consumption when the deep body temperature decreases, after the administration compared with before the administration, and a decrease in the estimated value of the deep body temperature when the oxygen consumption is assumed to be 0, after the administration compared with before the administration.
   (18) A method for treating a mammalian animal having a disease, the method comprising:
inducing a controlled hypometabolic state in the animal.
   (19) The method according to (18) above, wherein the inducing a controlled hypometabolic state in the mammalian animal is carried out in such a way as to slow progression of the disease in the mammalian animal.
   (20) The method according to (18) above, wherein
the inducing a controlled hypometabolic state in the mammalian animal is carried out in such a way as to decrease mortality of the mammalian animal.
   (21) The method according to any one of (18) to (20) above, wherein the controlled hypometabolic state is a hibernation-like state.
   (22) The method according to any one of (18) to (21) above, wherein the inducing a controlled hypometabolic state is carried out by stimulating a QRFP neuron.
   (23) The method according to any one of (18) to (22) above, wherein the disease is a serious acute disease.
   (24) The method according to any one of (18) to (23) above, wherein the method further comprises following up the animal.
   (25) The method according to (24) above, wherein the following up comprises measuring a change in oxygen consumption of the animal over time.
   (26) The method according to (24) or (25) above, wherein the following up comprises observing progression of the disease in the animal.
   (27) The method according to any one of (24) to (26) above, wherein the following up the animal is carried out during transport of the animal to a medical facility or in an intensive care unit of a medical facility.
   (28) A method for testing effectiveness of a treatment (for example, a treatment that induces a hypometabolic state), the method comprising:
subjecting a mammalian animal having a disease to the treatment;
following up the treated mammalian animal; and
determining any selected from whether or not the treatment decreases the rate of progression of the disease, whether or not the treatment decreases mortality, and whether or not the treatment prolongs survival time, compared with a group of a subject not receiving the treatment.
   (29) The apparatus according to any one of (1) to (6), (7) to (11), and (17) above, or the method according to any one of (12) to (16) and (18) to (28) above, wherein the disease is an acute disease.
   (30) The apparatus according to any one of (1) to (6), (7) to (11), and (17) above, or the method according to any one of (12) to (16) and (18) to (28) above, wherein the disease is a chronic disease.
   (31) The apparatus or the method according to (29) above, wherein the disease is a serious disease.
   (32) The apparatus or the method according to (30) above, wherein the disease is a serious disease.
   (33) A vehicle comprising the apparatus according to any one of (1) to (6), (7) to (11), and (17) above.
   (34) A vehicle for ambulance transport comprising the apparatus according to any one of (1) to (6), (7) to (11), and (17) above.

### BRIEF DESCRIPTION OF DRAWINGS

FIGS. 1a to 1h relate to activation of a Qrfp-iCre neuron that decreases hypothalamic body temperature and energy consumption. FIG. 1a shows a strategy for chemogenetic excitation of an iCre-positive neuron in a Qrfp-iCre mouse.

FIG. 1b Chemical excitation of an iCre-positive cell in Qrfp-iCre mice was found to induce hypothermia as a result of measurement by infrared thermography. Heterozygous (Q-het) or homozygous (Q-homo) Qrfp-iCre mice having a heterozygous Rosa26^{dreaddm3} (M3) and/or Rosa26^{dreaddm4} (M4) allele were subjected to an experiment. FIG. 1c shows a distribution of Q-neurons in the Qrfp-iCre mice. FIG. 1c is depicted by the expression of GFP after injecting AAV10-DIO-GFP into the medial basal hypothalamus. Scale bar (horizontal image), 500 μm; insertion portion, 100 μm; coronal image, 200 μm. Pe: periventricular nucleus, AVPe: anteroventricular Pe, MPA: medial preoptic area, LPO: lateral preoptic area, AHA: anterior hypothalamus, VMH: ventromedial hypothalamus, LHA: lateral hypothalamus, SON: supraoptic nucleus, DMH: dorsomedial hypothalamus, TMN: tuberomammillary nucleus, MM: medial mammillary nucleus, SCN: suprachiasmatic nucleus, VOLT: vascular organ of the lamina terminalis; TC, tuber cinereum; ARC, arcuate nucleus; opt, optic tract 3V, third ventricle.

FIG. 1d shows representative body temperature measurement results showing the surface body temperature of a Q-hM3D mouse. CNO was intraperitoneally injected at 0 hours. Note that the temperature of the tail rises at 0.5 hours (arrow).

FIG. 1e shows Fos immunostaining of sliced specimens from Q-hM3D mice 90 minutes after CNO IP. Scale bar, 100 μm.

FIG. 1f shows the procedure for metabolic analysis by chemogenetic activation of a Q-neuron in a Q-hM3D mouse.

FIG. 1g shows the progression of hypothermia/hypometabolism over time after DREADD-mediated activation of a Cre-positive neuron. Purple line, Q-hM3D mice; yellow line, Qrfp-iCre mice injected with AAV10-DIO-hM3Dq-mCherry into the lateral hypothalamus; black line, Orfp-iCre mice injected with AAV-DIO-mCherry into the medial basal hypothalamus (negative control).

FIG. 1h Q-neuron-induced hypometabolism (QIH) can last for several days and can be induced again by a CNO injection. The lines and shading in b and g represent the average value and the standard deviation, respectively, of each group.

FIGS. 2a to 2l show results of histological and functional analyses of Q-neuron projections. FIG. 2a shows a strategy for visualizing an axonal projection pattern of Q-neurons visualized by expressing GFP in the Q-neurons by injecting AAV-DIO-GFP into a Qrfp-iCre mouse.

FIG. 2b shows distributions of GFP-positive Q-neurons in AVPe, MPA, and Pe. Scale bar, 100 μm. FIG. 2c shows distributions of axons generated from Q-neurons. Scale bar, 100 μm.

FIG. 2d A cropped image of an image of the brain captured by a ScaleS method was clarified by the Scales method, and Q-neurons in AVPe and fibers in DMH are shown.

FIG. 2e shows an in situ hybridization analysis showing that a population of Q-neurons expresses Vgat and/or Vglut2 in a Q-hM3D mouse. Scale bar, 100 μm. FIG. 2f shows a high-magnification image of the rectangular region shown in FIG. 2e.

FIG. 2g shows a monochrome image of the rectangular region in FIG. 2e.

FIG. 2h shows high-magnification images of the rectangular regions 1 to 3 shown in FIG. 2f, which show Q-neurons expressing Vgat, Vglut2, or both. (1) Vgat⁺mCherry⁺; (2) Vglt2⁺mCherry⁺; (3) Vgat⁺Vglt2⁺mCherry⁺.

FIG. 2i shows the proportion of Vgat-positive neurons (1291 out of 1997 cells), Vglut2 (359 out of 1997 cells), and (115 out of 1197 cells) in mCherry-expressing cells (counted in four sections prepared from two mice). Other mCherry-expressing cells do not express Vgat or Vglut2.

FIG. 2j shows a strategy for optogenetic excitation of Q-neurons or their axons in DMH and RPa; scale bar, 100 μm.

FIG. 2k shows changes in body temperature measured with a thermographic camera during optogenetic excitation in DMH or RPa of Cre-positive cells or their axons in AVPe/MPA. Four shots of light stimulation are shown by blue arrowheads. The lines and shading show the average value and the standard deviation, respectively, of each group. The lower panels show representative thermographic images obtained by excitation of Q-neurons (AVPe/MPA). Note that the tail exhibits heat release after 5 minutes from the first light stimulation (arrow).

FIG. 2l shows an estimated Tₛ 30 minutes after the fourth light stimulation. It should be noted that the effect of DMH fiber stimulation on Ts is almost comparable to the effect of excitation of a cell body in AVPe/MPA. Pe, periventricular nucleus; AVPe, anteroventricular Pe; VOLT, vascular organ of the lamina terminalis; MPA, medial preoptic area; VLPO, ventrolateral preoptic area; PVN, paraventricular hypothalamic nucleus; SON, supraoptic nucleus; DMH, dorsomedial hypothalamus; TMN, tuberomammillary nucleus; MM, medial mammillary nucleus; LC, locus coeruleus; PAG, periaqueductal grey; LPB, lateral parabrachial nucleus; RVLM, rostral ventrolateral medulla; RPa, raphe pallidus nucleus; 3V, third ventricle.

FIG. 3a Hypometabolism induced by Q-neurons is accompanied by a decreased setpoint of body temperature. Changes in T_{B} and VO₂ in QIH at various T_{A}s. QIH was induced in Q-hM3D mice by CNO injection. The lines and shading show the average value and the standard deviation, respectively, of each group.

FIG. 3b shows the minimum T_{B} (left) and VO₂ (right) under normal and QIH conditions.

FIG. 3c shows a schematic view of heat production and heat loss pathways in a mammal. Heat loss is proportional to a difference between T_{A} and T_{B} at factor G. Heat production is governed by a difference between T_{R} and T_{A} at factor H.

FIG. 3d shows the relationship between T_{B}−T_{A} and VO₂ at various TAS. The slope of the curve represents G. The dots are recorded data, thick lines are drawn from the median of posterior G, and thin lines are curves drawn from 500 G randomly selected from posterior samples.

FIG. 3e shows a posterior distribution of estimated G (e) and a difference in G from QIH to the normal state (f).

FIG. 3f shows a posterior distribution of estimated G (e) and a difference in G from QIH to the normal state (f).

FIG. 3g shows the relationship between T_{B} and VO₂ at various TAS. The negative slope of the curve represents H, and the x-intercept represents T_{R}. See FIG. 3d for a description of the dots and the lines.

FIG. 3h shows a distribution of estimated H (h) and a difference in H from QIH to the normal state (i).

FIG. 3i shows a distribution of estimated H (h) and a difference in H from QIH to the normal state (i).

FIG. 3j shows a distribution of estimated TR (j) and a difference in TR from QIH to the normal state (k).

FIG. 3k shows a distribution of estimated TR (j) and a difference in TR from QIH to the normal state (k).

FIG. 3l shows a metabolic transition of QIH within an individual. The upper row shows transition of animal posture at various TAS during QIH. The second row is timewise magnification of the third row, both showing metabolic transitions in one representative animal. Note that the mouse shows a curled-up posture during FIT at T_{A}=28° C. (B) and shows an extended posture during QIH at T_{A}=28° C. (D). Even during QIH when T_{A} was lowered to 12° C., the animal assumed a curled-up posture, as in FIT (E), showing that the animal assumed a posture for avoiding heat loss.

FIGS. 4a to 4g show that Q-neurons serve to induce fasting-induced torpor in mice. FIG. 4a shows a strategy for suppressing the function of Q-neurons. Left panel, experimental procedure. Right panels, expression of TeTxLC-eYFP in AVPe/MPA shown by immunostaining with anti-GFP antibody. Scale bar, 100 μm.

FIG. 4b shows a schematic view of an FIT experiment.

FIG. 4c shows that normal FIT was no longer caused by expressing TeTxLC in a Q-neuron. Note that a rapid oscillatory decrease in metabolism was not seen in these mice.

FIG. 4d The Minimum VO₂ during 24 to 36 hours and 36 to 48 hours was compared between control mice and TeTxLC mice. Suppression of a Q-neuron blocked the decrease in VO₂ normally seen in FIT. The estimated difference in minimum VO₂ between the control group and the TeTxLC mice was [0.01, 0.80] ml/g/h during 24 to 36 hours and [0.36, 1.16] ml/g/h during 36 to 48 hours. The smaller SD of T_{B} and VO₂ in the TeTxLC mice shows that a Q-neuron is involved in an abrupt change in metabolism including an oscillatory change during FIT. The “>” and “<” symbols represent whether either the 89% HPDI of the estimated difference in the minimum value or the standard deviation from TeTxLC to the control mice is negative or positive, respectively.

FIG. 4e shows that FIT was induced in the control, Qrfp-iCre heterozygous mice and homozygous mice, showing that lack of a QRFP peptide did not affect FIT.

FIG. 4f shows the procedure for visualizing input neurons that come into monosynaptic contact with Q-neurons by using a recombinant rabies virus vector.

FIG. 4g shows distributions of input neurons of Q-neurons. Arrows show starter cells. Scale bar, 100 μm.

FIG. 4h shows brain regions including input neurons. Scale bar, 100 μm. Pe, periventricular nucleus; AVPe, anteroventricular Pe; MPA, medial preoptic area; VOLT, vascular organ of the lamina terminalis; MnPO, median preoptic area; VMPO, ventromedial preoptic area; VLPO, ventrolateral preoptic area; PVN, paraventricular hypothalamic nucleus; TC, tuber; opt, optic tract; ac, anterior commissure; f, fornix; 3V, third ventricle.

FIG. 5 shows an overview of the apparatus according to a first embodiment.

FIG. 6 shows an overview of the apparatus according to the first embodiment.

FIG. 7 shows an overview of the apparatus according to a second embodiment.

FIG. 8 shows an overview of additional configurations of the apparatuses of the first and second embodiments.

FIG. 9A shows a scheme of an experiment involving observing a sepsis model animal for 48 hours with or without induction of hibernation (QIH).

FIG. 9B shows changes over time in survival rates of sepsis model animals in which hibernation (QIH) was induced and sepsis model animals in which hibernation was not induced.

FIG. 10 shows oxygen consumption graphs in a sepsis model, a hypometabolism-induced sepsis model, and a typical QIH (hibernation) model.

FIG. 11A shows a scheme of an experiment involving observing an acute renal failure model animal for 48 hours with or without induction of hibernation (QIH).

FIG. 11B shows changes over time in survival rates of acute renal failure model animals in which hibernation (QIH) was induced and acute renal failure model animals in which hibernation was not induced.

FIG. 12A shows an example of the configuration of the apparatus (C) of the present invention.

FIG. 12B shows an example of the configuration of the apparatus (C) of the present invention.

FIG. 12C shows a flow chart for determining the possibility of a hibernation-like state and determining a dead state in the present invention.

FIG. 12D shows a determination flow chart for determining whether, when a specified range is determined from a fluctuation in oxygen consumption in a subject in a stable metabolic state, the oxygen consumption of the subject is within or outside the range, in the present invention.

FIG. 12E shows an example of the configuration of the apparatus (C) of the present invention.

### SPECIFIC DESCRIPTION OF INVENTION

As used herein, the “subject” refers to a human and a non-human mammalian animal, for example, a rodent such as a mouse, a rat, a guinea pig, a gerbil, or a hamster, a laboratory animal such as a ferret, a rabbit, a dog, or a minipig, and a non-human primate such as a monkey, a gorilla, a chimpanzee, an orangutan, and a bonobo.

In an embodiment, the subject has a disease. In an embodiment, the subject has a chronic disease. In an embodiment, the subject has an acute disease. In an embodiment, the disease, the chronic disease, or the acute disease is a serious disease. In all embodiments of the invention, the subject is preferably a subject having a disease. The acute disease refers to a disease in which a symptom develops abruptly and progresses rapidly. Examples of the acute disease include sepsis, acute renal failure, acute pneumonia, myocardial infarction, cerebral infarction, cerebral hemorrhage, and hemorrhagic shock. The chronic disease refers to a disease other than an acute disease. Examples of the chronic disease include a tumor, an autoimmune disease, and aging.

As used herein, the “hypothalamus” is a center that is present in the diencephalon and regulates endocrine and autonomic functions. As used herein, the “Q-neuron” is a neuron that is present in the medial regions of the hypothalamus, that is, the anteroventral periventricular nucleus (AVPe), the medial preoptic area (MPA), and the periventricular nucleus (Pe), and this neuron produces pyroglutamylated RFamide peptide (QRFP). The pyroglutamylated RFamide peptide (QRFP) is a neuropeptide identified as an endogenous ligand for the GPR103 receptor. QRFP is strongly expressed in the hypothalamus, and is thought to be involved in the regulation of sleep and wakefulness as QRFP has been shown to have the effect of enhancing the wakefulness system.

As used herein, “T_{A}” means the ambient environment temperature (° C.) of the subject, “T_{B}” means a deep body temperature (° C.), and “T_{R}” means a theoretical setpoint temperature (° C.). “VO₂” means the oxygen consumption of the subject. T_{R} is the body temperature determined as T_{B} when the correlation between T_{B} and VO₂ when T_{A} is varied is determined and VO₂ is zero. T_{B} is not the body surface temperature, which is affected by the outside air temperature, but the internal body temperature. For example, T_{B} in a human can be defined by rectal, intraesophageal, intravesical, or intrapulmonary blood temperature. The negative feedback gain (H) of heat generation shows heat generation efficiency and is determined by H=ΔVO₂/ΔT_{B}.

As used herein, the “hypometabolic state” is a controlled hypometabolic state and includes a hibernation-like state. A controlled hypometabolic state can be induced by hypometabolic therapy. Hypometabolic therapy induces a state in which metabolism is decreased by decreasing the oxygen consumption required by a tissue. In addition, the hypometabolic state is preferably controlled by the brain. The hypometabolic state controlled by the brain can be induced by stimulating a Q-neuron in the brain. In addition, the hypometabolic state controlled by the brain can be induced by the method of the present invention. Examples of the hypometabolic therapy include the induction of natural torpor (for example, daily torpor, hibernation, or Q-neuron-induced hypometabolism (QIH)) and a state similar thereto. Examples of a method for inducing a short-term hypometabolic state (torpor) include a method involving limiting the amount of food (for example, starvation under a condition of an outside air temperature of 12 to 24° C. (for example, withdrawing food for 24 hours)). A short-term hypometabolic state can preferably be induced in a rodent such as a mouse or a Syrian hamster. Examples of a method for inducing a longer-term hypometabolic state (hibernation) include a method involving stimulating a Q-neuron. Stimulation of a Q-neuron can be effective in order to induce a hypometabolic state in a mammalian animal that does not hibernate in nature (non-hibernating mammalian animal). Hypothermia is secondary in hypometabolic therapy. In contrast, hypothermic therapy decreases body temperature by a method that directly decreases body temperature. Hypothermic therapy is a therapy that directly decreases body temperature, for example, by rapid intravenous injection of an agent cooled to 4° C., and thus is essentially different from hypometabolic therapy. In particular, a mammal tries to keep the body temperature thereof constant, thus cooling the body as in hypothermic therapy requires extra metabolism and rather tends to increase metabolism, unless a muscle relaxant is used to suppress shivering. Therefore, hypothermic therapy is essentially different from hypometabolic therapy.

As used herein, “hibernation” is a hypothermic and hypometabolic state found in a mammalian animal. “Daily torpor” is a short-term hypometabolic state. Hibernation and daily torpor are different from each other in that in daily torpor, a decrease in H occurs with almost no decrease in T_{R}, whereas in hibernation, both T_{R} and H significantly decrease. As used herein, the “hibernation-like state” means a state in which both T_{R} and H significantly decrease with a decrease in T_{A}. As used herein, the “non-hibernating animal” refers to an animal that does not have a habit of hibernating in winter or during fasting.

As used herein, “exhaled air” is breath exhaled by a subject. As used herein, oxygen concentration is an index representing the amount of oxygen per volume. The unit of oxygen concentration can be, for example, % or mmHg. As used herein, “oxygen consumption” (VO₂) is the amount of oxygen consumed per hour calculated from the concentration of oxygen included in exhaled air and inhaled air. Oxygen consumption varies with body weight, and thus may be corrected and calculated per unit body weight (for example, per kg and per g). Oxygen consumption can be calculated per unit time (for example, per minute or per hour).

The present inventors have found that by applying an excitatory stimulus to a pyroglutamylated RFamide peptide (QRFP)-producing neuron in one or more regions selected from the group consisting of the anteroventral periventricular nucleus (AVPe), the medial preoptic area (MPA), and the periventricular nucleus (Pe) of the hypothalamus in the brain of a living, non-hibernating animal, a hibernation-like state can be induced in the subject. The present inventors have also found that when a subject has a disease, induction of a hypometabolic state (preferably a hibernation-like state) improves the survival rate of the subject. By inducing a hypometabolic state (preferably a hibernation-like state), the metabolism of a subject can be decreased, thereby slowing all vital activities, including progression of the disease. Therefore, in the present invention, the subject is a subject having a disease, particularly a disease affecting the survival rate (for example, serious disease).

As used herein, “induction of a hypometabolic state in a peripheral tissue” refers to inducing a hypometabolic state in a peripheral tissue to decrease oxygen demand and nutrient demand in the peripheral tissue. The induction of a hypometabolic state in an individual in the invention can be accompanied by induction of a hypometabolic state in a peripheral tissue. For example, QIH is accompanied by induction of a hypometabolic state in a peripheral tissue, and thus in QIH, the oxygen consumption in an individual decreases, the respiration decreases, and the heart rate also decreases. Without being bound by theory, this decrease in respiration and heart rate can be achieved by the brain controlling the respiration and the heart rate to meet the oxygen and nutrient demand of a peripheral tissue. In contrast, anesthetic treatment suppresses the entire brain and causes the brain to lose control itself, and thus anesthesia is completely different in the controlled state of the body (and a peripheral tissue) by the brain from the hypometabolic state in QIH. In contrast, in hypothermic therapy, the body temperature of a mammalian animal is forcibly decreased. Hypothermic therapy can cause a hypometabolic state due to forced hypothermia induction to occur in a peripheral tissue, but cannot decrease metabolism as much as hypometabolic therapy. In addition, it is known that hypothermic therapy instead causes shivering in muscle tissue and rather increases metabolism in the absence of a muscle relaxant. In contrast, the hypometabolic state due to hypometabolic therapy results in decreased oxygen consumption in a peripheral tissue, which causes the brain to slow down respiration and circulation and can decrease metabolism beyond what is possible with hypothermic therapy. As described above, in hypometabolic therapy, active control by the brain produces a hypometabolic state and a hypothermic state in a peripheral tissue, and in this respect, the hypometabolic state due to hypometabolic therapy is different from the hypometabolic state due to anesthesia or hypothermic therapy without such control by the brain.

Therefore, according to the present inventors, provided is a method for inducing a hibernation-like state in a subject that is a living, non-hibernating animal, the method including applying an excitatory stimulus to a pyroglutamylated RFamide peptide (QRFP)-producing neuron in one or more regions selected from the group consisting of the anteroventral periventricular nucleus (AVPe), the medial preoptic area (MPA), and the periventricular nucleus (Pe) of the hypothalamus. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The serious disease is a disease that is life-threatening if treatment thereof is delayed. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

An excitatory stimulus can be caused by stimulating by using a deep brain electrode or by stimulating by using an activating agent for a QRFP-producing neuron.

According to the present invention, provided is an apparatus that stimulates a pyroglutamylated RFamide peptide (QRFP)-producing neuron in one or more regions selected from the group consisting of the anteroventral periventricular nucleus (AVPe), the medial preoptic area (MPA), and the periventricular nucleus (Pe) in the brain of a living subject (hereinafter sometimes referred to as the “apparatus of the present invention”).

(A1) The apparatus of the present invention can include:
a control unit that transmits a control signal controlling voltage generation;
a voltage generation unit that receives a control signal from the control unit and generates a voltage; and
a stimulation probe electrically connected proximally to the voltage generation unit and including an electrical stimulation electrode distally wherein the stimulation probe has a length sufficient to access a QRFP-producing neuron from a brain surface, and generates an electrical stimulus at a distal electrical stimulation electrode with the voltage from the voltage generation unit. Thereby, the apparatus of the present invention can electrically provide an excitatory stimulus to a QRFP-producing neuron. Alternatively, from the viewpoint of providing a chemical stimulus instead of an electrical stimulus,
   (A2) the apparatus of the present invention can include:
a control unit that transmits a control signal controlling release of a QRFP-producing neuron-stimulating compound;
a storage unit for the compound; and
a compound release unit that receives the control signal from the control unit and releases the compound from the storage unit for the compound.
   (B) The apparatus of the present invention may further include:
an outside air temperature gauge;
a deep body temperature;
an exhaled gas analysis unit that measures an oxygen concentration of exhaled gas; and
a recording unit that records a measured outside air temperature and at least one numerical value selected from the group consisting of a deep body temperature and the oxygen concentration. In configuration (B) of the apparatus of the present invention, it can be checked whether or not the deep body temperature (T_{B}) decreases with a decrease in outside air temperature (T_{A}) in the subject, and the oxygen consumption of the subject can be determined from exhaled gas analysis results to determine the theoretical setpoint temperature (T_{R}) and the negative feedback gain (H) of heat generation. Thereby, the apparatus of the present invention can determine whether or not the subject has induced a hibernation-like state.

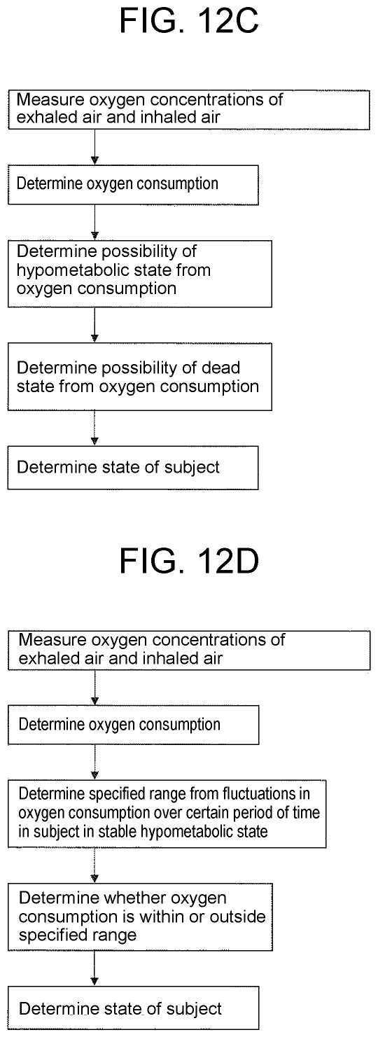

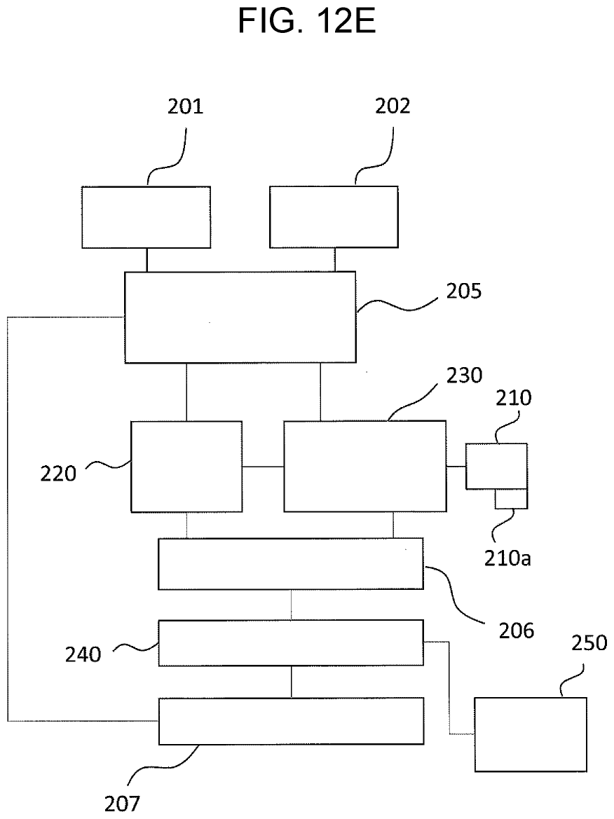

The apparatus (C) of the present invention, as shown in FIG. 12A, may further include:
a computation unit 205 that calculates an oxygen consumption of a subject from an oxygen concentration difference between exhaled air and inhaled air from the subject;
a hypometabolic state (preferably a hibernation-like state) possibility determination unit 206 that determines that the subject has entered a hypometabolic state (preferably a hibernation-like state) or may have entered a hypometabolic state (preferably a hibernation-like state) at least based on a decrease in oxygen consumption of the subject compared with an oxygen consumption of the subject before entering the hypometabolic state (preferably a hibernation-like state); and/or
a death possibility determination unit 207 that determines that the subject is dead or may be dead at least based on the oxygen consumption of the subject, a carbon dioxide concentration of the exhaled air of the subject, an electrocardiographic waveform of the subject, or a blood pressure of the subject of zero. The carbon dioxide concentration of the exhaled air of the subject, the electrocardiographic waveform of the subject, or data on the blood pressure of the subject can be input to the apparatus of the present invention by an emergency monitor that acquires an electrocardiogram and the blood pressure of the subject. In addition, the carbon dioxide concentration of the exhaled air of the subject can be input to the apparatus of the present invention by an exhaled air carbon dioxide partial pressure monitor. The apparatus (C) may further include a determination unit 240 that determines whether the subject is in a hibernating state or in a dead state by inputting the respective determinations from the hypometabolic state (preferably a hibernation-like state) possibility determination unit 206 and the death possibility determination unit 207. The determination unit 240 can be connected to an output device 250 (for example, a display or a speaker), and a result can be output from the output device 250. Therefore, the apparatus (C) may further include an exhaled air measurement unit 201 that measures the oxygen concentration of the exhaled air and an inhaled air measurement unit 202 that measures the oxygen concentration of the inhaled air. The determination unit 240 may determine the state of the subject, for example, according to the flowchart shown in FIG. 12C. The apparatus (C) of the present invention may record a fluctuation in oxygen consumption over a certain period of time in the subject in a stable hypometabolic state (preferably a hibernation-like state), and when the fluctuation falls outside the specified range, may output a notification from the output device 250. Here, when the fluctuation is within the specified range, the following evaluation can be provided: the stable hypometabolic state is maintained, and when the fluctuation is outside the specified range, the following evaluation can be provided: the subject has escaped from the stable hypometabolic state. The specified range can be determined based on a fluctuation in oxygen consumption over a certain period of time. For example, it is possible to obtain an average value of fluctuations in oxygen consumption for a certain period of time, and set the range from average value×lower preset value to average value×upper preset value as the specified range. The lower preset value can be, for example, 0.7 or less, 0.6 or less, 0.5 or less, 0.4 or less, 0.3 or less, 0.2 or less, or 0.1 or less. The upper preset value may also be, for example, 1.3 or more, 1.4 or more, 1.5 or more, 1.6 or more, 1.7 or more, 1.8 or more, 1.9 or more, 2 or more, 2.1 or more, 2.2 or more, 2.3 or more, 2.4 or more, 2.5 or more, 2.6 or more, 2.7 or more, 2.8 or more, 2.9 or more, or 3 or more. The lower and upper default values are 0.7 and 1.3, 0.6 and 1.4, 0.5 and 1.5, 0.4 and 1.6, 0.3 and 1.7, 0.2 and 1.8, and 0.1 and 1.9, respectively. Because of this, the apparatus (C) may further include a determination unit 241 that records a fluctuation in oxygen consumption over a certain period of time in the subject in a stable hypometabolic state (preferably a hibernation-like state), sets a specified range based on the fluctuation in oxygen consumption over a certain period of time (for example, the upper limit value and the lower limit value, or the average value, of the oxygen consumption in the certain period of time), and determines whether the oxygen consumption is within or outside the specified range. For example, the upper limit value of the specified range can be determined based on the upper limit value of the fluctuation in oxygen consumption over the certain period of time. For example, the upper limit value of the specified range can be set to be a first proportion higher than the upper limit value of the fluctuation in oxygen consumption over the certain period of time, and the first proportion can be 5% or more, 10% or more, 15% or more, 20% or more, 25% or more, 30% or more, 40% or more, 50% or more, 60% or more, 70% or more, 80% or more, 90% or more, or 100% or more. For example, the lower limit value of the specified range can be determined based on the lower limit value of the fluctuation in oxygen consumption over the certain period of time. For example, the lower limit value of the specified range can be set to be a second proportion lower than the lower limit value of the fluctuation in oxygen consumption over the certain period of time, and the second proportion can be 5% or more, 10% or more, 15% or more, 20% or more, 25% or more, 30% or more, 40% or more, 50% or more, 60% or more, 70% or more, 80% or more, 90% or more, or 100% or more. When the oxygen consumption exceeds the upper limit of the specified range, this means that the subject may be waking up from the controlled hypometabolic state, and when the oxygen consumption falls below the lower limit of the specified range, this means that the subject may be transitioning from the controlled hypometabolic state to greater hypometabolism (for example, be dying). Therefore, the determination result of whether the oxygen consumption is within or outside the specified range in the determination unit 241 is preferably output to the output device 250. The determination unit 241 may determine the state of the subject, for example, according to the flowchart shown in FIG. 12D. The determination results of the determination unit 240 and the determination result of the determination unit 241 may be displayed on the output device 250 at the same time. Here, when the fluctuation in oxygen consumption of the subject in a hypometabolic state is within the range of the above fluctuation in oxygen consumption over the certain period of time±20%, within the range±15%, within the range±10%, or within the range±5%, the following evaluation can be provided: the subject is in a stable hypometabolic state.

In an embodiment, the apparatus (C) includes an arithmetic circuit 260 (for example, a processor) that can execute:
an oxygen consumption calculation program that calculates an oxygen consumption of the subject based on an oxygen concentration difference between exhaled air and inhaled air from the subject;
a hypometabolic state possibility determination program that determines that the subject has entered a hypometabolic state (preferably a hibernation-like state) or may have entered a hypometabolic state (preferably a hibernation-like state) at least based on a decrease in oxygen consumption compared with an oxygen consumption of the subject before entering the hypometabolic state (preferably a hibernation-like state); and
a death possibility determination program that determines that the subject is dead or may be dead at least based on the oxygen consumption of the subject, a carbon dioxide concentration of the exhaled air of the subject, an electrocardiogram of the subject, or a blood pressure of the subject of zero. The apparatus (C) can transmit a determination result from the arithmetic circuit to the output device 250, and the output device 250 can output the result. In a preferable embodiment, for example, as shown in FIG. 12D, the arithmetic circuit 260 of the apparatus (C) may further execute a program that records a fluctuation in oxygen consumption of the subject in a stable hypometabolic state (preferably a hibernation-like state) over a certain period of time, determines a specified range, and then determines whether the oxygen consumption is within or outside the specified range. The arithmetic circuit 260 of the apparatus (C) can output, from the output device 250, a determination result of the program that determines whether the oxygen consumption is within or outside the specified range. The output result is, for example, displayed on a display, or output as a sound (for example, a warning sound) from a speaker, or displayed on a display and output as a sound (for example, warning sound) from a speaker. These programs can be executed by one or more arithmetic circuits.

In an embodiment, the apparatus (C) can be an apparatus including:
one or more processors;
a memory; and
one or more programs, wherein the one or more programs are stored in the memory and configured to be executable by the one or more processors, and the one or more programs include:
an oxygen consumption calculation program that calculates an oxygen consumption of a subject based on an oxygen concentration difference between exhaled air and inhaled air from the subject;
a hypometabolic state possibility determination program that determines that the subject has entered a hypometabolic state (preferably a hibernation-like state) or may have entered a hypometabolic state (preferably a hibernation-like state) at least based on a decrease in oxygen consumption compared with an oxygen consumption of the subject before entering the hypometabolic state (preferably a hibernation-like state); and
a death possibility determination program that determines that the subject is dead or may be dead at least based on the oxygen consumption of the subject, a carbon dioxide concentration of the exhaled air of the subject, an electrocardiogramaveform of the subject, or a blood pressure of the subject of zero.
   (C′) The apparatus of the present invention, as shown in FIG. 12B, may further include:
a computation unit 205 that calculates an oxygen consumption of a subject from an oxygen concentration difference between exhaled air and inhaled air from the subject;
a hibernation possibility determination unit 206 that determines that the subject has entered a hibernation-like state or may have entered a hibernation-like state at least based on a decrease in both oxygen consumption and theoretical setpoint temperature compared with an oxygen consumption and a theoretical setpoint temperature of the subject in a non-hibernation-like state; and/or
a death possibility determination unit 207 that determines that the subject is dead or may be dead at least based on the oxygen consumption, a carbon dioxide concentration of the exhaled air of the subject, an electrocardiogram of the subject, or a blood pressure of the subject of zero. The apparatus (C) may further include a computation unit 220 that calculates the theoretical setpoint temperature. The theoretical setpoint temperature (T_{R}) of body temperature is determined as an estimated value of a deep body temperature (T_{B}) when the relationship between the deep body temperature (T_{B}) and an oxygen consumption (VO₂) is determined while varying (for example, decreasing) the outside air temperature (or ambient temperature of the subject) (T_{A}) and the oxygen consumption (VO₂) is zero. Therefore, the apparatus (C) may further include a deep body thermometer 210 and a body temperature storage unit 210a that stores a body temperature measured by the thermometer. In the apparatus (C), the computation unit 220 that calculates the theoretical setpoint temperature can calculate, as the theoretical setpoint temperature of the subject, an estimated value of the deep body temperature (T_{B}) when the oxygen consumption (VO₂) is 0 from the relationship between the deep body temperature and the oxygen consumption of the subject stored. The relationship between the deep body temperature (T_{B}) and the oxygen consumption (VO₂) is determined by curve fitting to obtain a fitted curve, and in an embodiment, the fitted curve can be linear. In addition, according to the present invention, the feedback gain (H) of heat generation can be determined as H=ΔVO₂/ΔT_{B}. Therefore, the apparatus (C) may further include a computation unit 230 that calculates the feedback gain (H) of heat generation. The apparatus (C) may further include a determination unit 240 that determines whether the subject is in a hibernating state or in a dead state by inputting the respective determinations from the hibernation possibility determination unit 206 and the death possibility determination unit 207. The determination unit 240 can be connected to the output device 250, and a result can be output from the output device 250. Therefore, in an embodiment, the apparatus (C) may further include a deep body thermometer, a body temperature storage unit that stores a body temperature measured by the thermometer, a computation unit that calculates the theoretical setpoint temperature, and a computation unit that calculates the feedback gain (H) of heat generation. The apparatus (C) may further include an exhaled air measurement unit 201 that measures the oxygen concentration of the exhaled air and an inhaled air measurement unit 202 that measures the oxygen concentration of the inhaled air.

Hereinafter, the apparatus of the present invention will be specifically described.

### First Embodiment

In a first embodiment, the apparatus of the present invention has the configuration of (A1) above. Thereby, the apparatus of the present invention electrically stimulates a QRFP-producing neuron in the brain of a living subject to induce a hibernation-like state in the subject. Hereinafter, the first embodiment will be described with reference to FIGS. 5 and 6.

An apparatus 1 of the present invention include:
a control unit 10 that transmits a control signal controlling voltage generation;
a voltage generation unit 20 that receives the control signal from the control unit and generates a voltage; and
a stimulation probe 30 electrically connected proximally to the voltage generation unit and including an electrical stimulation electrode distally wherein the stimulation probe has a length sufficient to access a QRFP-producing neuron from a brain surface, and generates an electrical stimulus at a distal electrical stimulation electrode 40 with the voltage from the voltage generation unit.

In the apparatus 1 of the present invention, the control section 10 transmits a control signal controlling voltage generation. The control unit 10 can include a control element (a microprocessor and a power supply or a battery). The control signal can control one or more instances of voltage generation with one control signal. Alternatively, this control signal can be transmitted a plurality of times to control a plurality of instances of voltage generation. The control signal can apply one voltage stimulus, and for example, the control signal may control voltage generation such that a stimulus is applied a plurality of times until a hibernation-like state is induced in the subject {however, after the induction of the hibernation-like state, a stimulus may or may not be applied}.

In the apparatus 1 of the present invention, the voltage generation unit 20 is electrically coupled to the control unit 10 through a wiring 15, and can receive a control signal from the control unit 10 to generate a voltage. The voltage can be, for example, a voltage of 0 to 5 volts (V), and can be varied, for example, in steps of 0.1 volts. The voltage can be, for example, a pulse voltage, the pulse width can be, for example, several tens of microseconds, and the stimulation frequency can be, for example, tens to hundreds of pps. The voltage may, for example, start at 1 volt and be adjusted in such a way as to increase until an effect is observed.

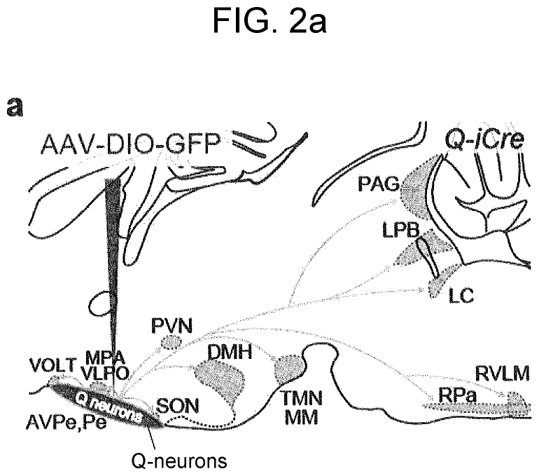

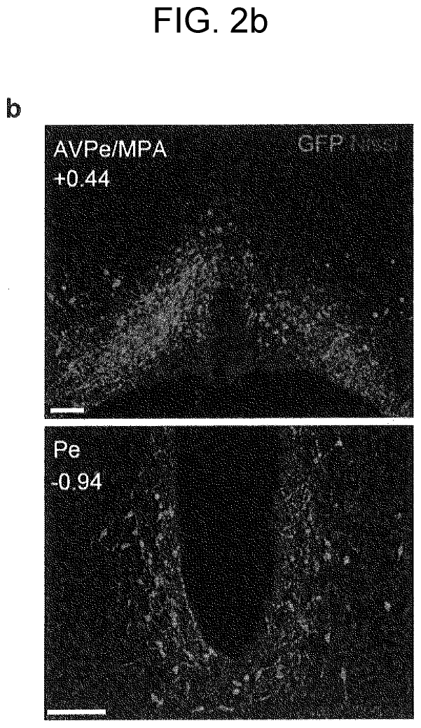

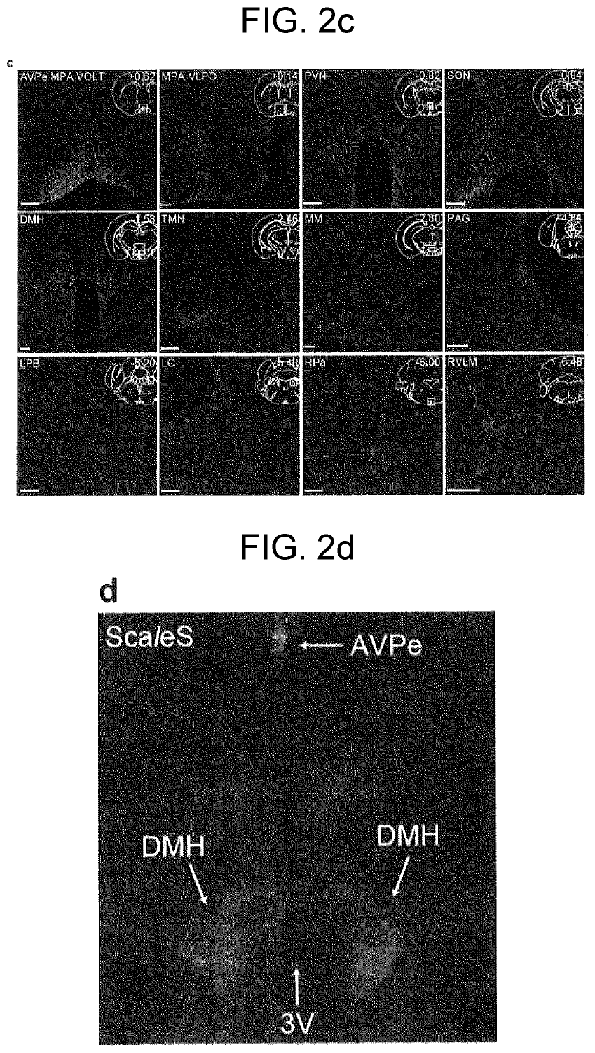

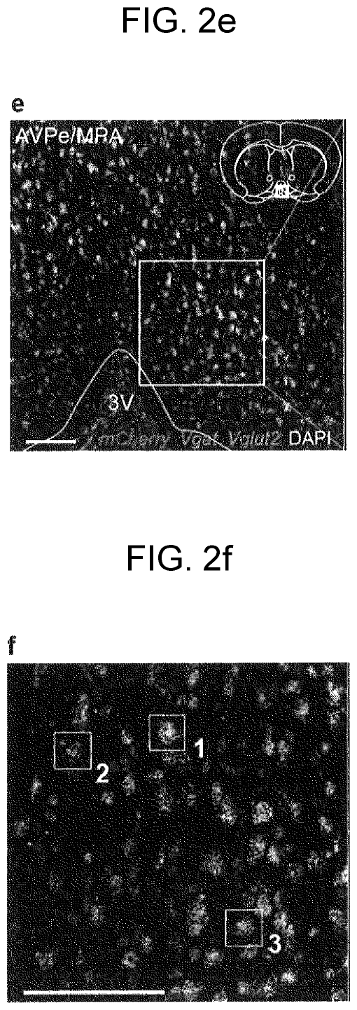

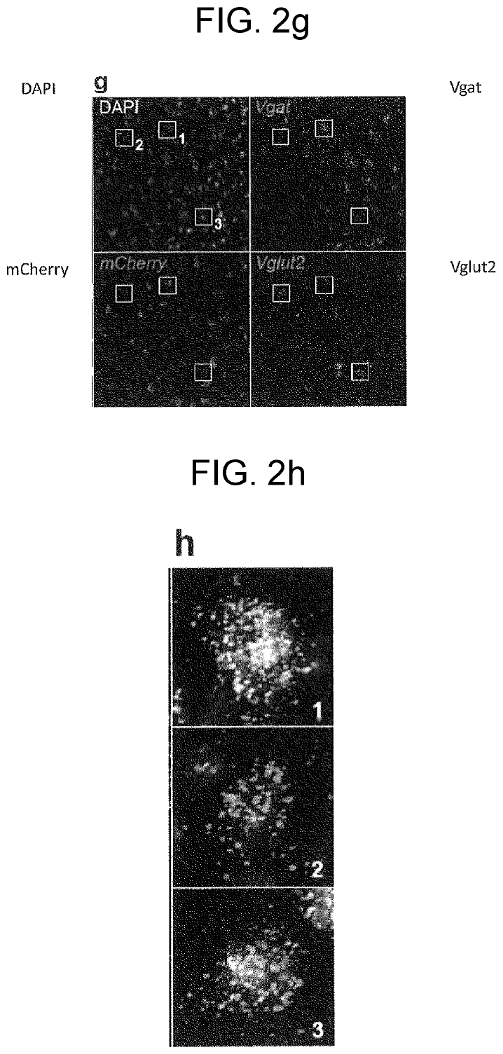

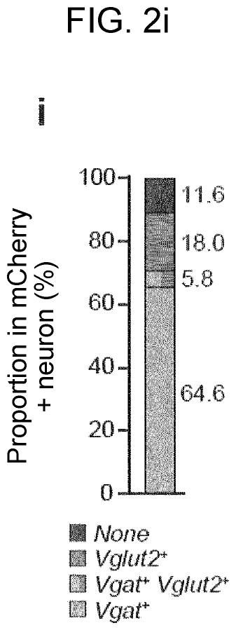

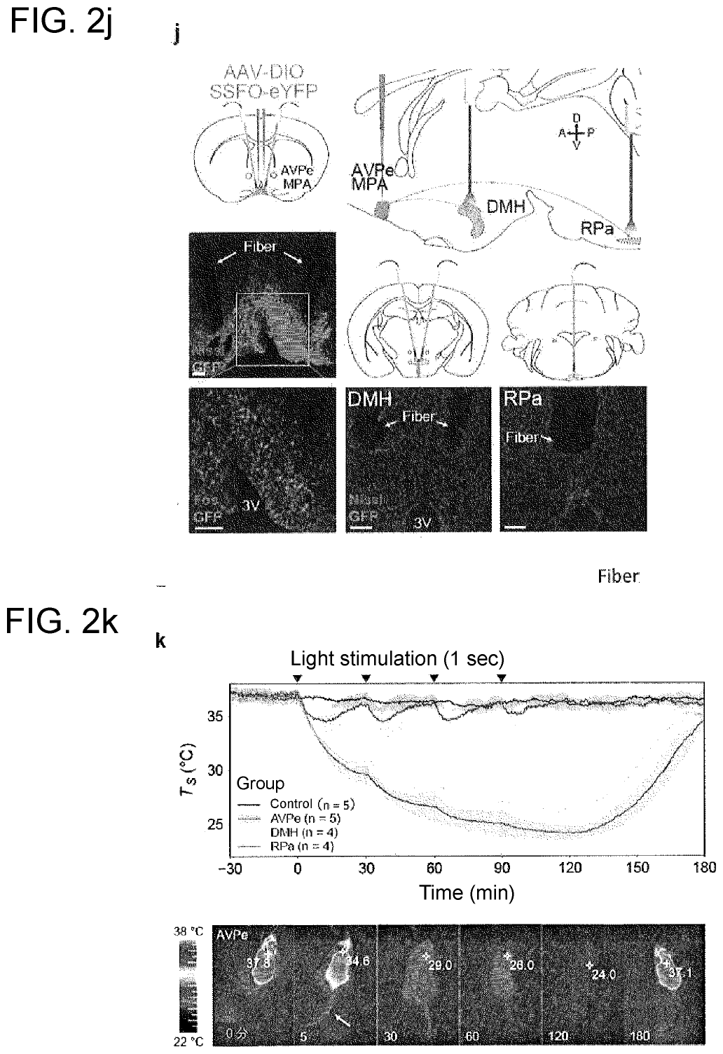

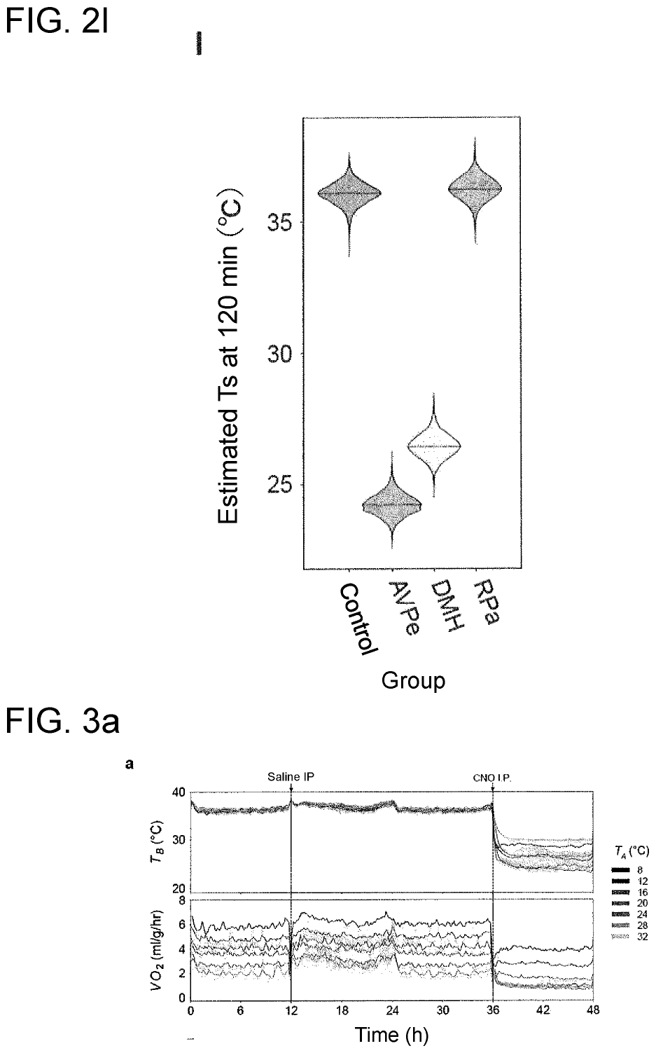

An example in which the control unit 10 and the voltage generation unit 20 are coupled through the wiring 15 was described, and in the apparatus 1 of the present invention, instead of the wiring 15, as shown in FIG. 2, the control unit 10 and the voltage generation unit 20 may be able to wirelessly communicate between a control signal transmission unit 11 included in the control unit 10 and a control signal reception unit 21 included in the voltage generation unit. In this embodiment, the voltage generation unit 20 can have a battery 20a. The battery 20a can be rechargeable in a non-contact manner. When charging is possible in a non-contact manner, the battery 20a can be charged from outside the body even when present in the body.

The voltage generation unit 20 transmits a voltage generated by the voltage generation unit 20 to the stimulation probe 30 and the stimulation electrode 40 present at the tip thereof, through an extension cable 25. The distal (that is, the tip) of the stimulation probe 30 has the stimulation electrode 40, and the stimulation electrode 40 can apply a voltage to the tissue of the brain.

The stimulation probe 30 can be inserted into the brain by stereotaxic surgery in order to cause the stimulation electrode 40 to accurately reach a QRFP-producing neuron. Stereotaxic surgery is a surgery in which the head is fixed with a measurement frame, and an electrode is inserted with a precision of 1 mm or less into a position at which the electrode is to be inserted, the position being determined by a CT scan or MRI. From the viewpoint of stereotaxic surgery, the stimulation probe 30 is formed from a material that is hard enough not to cause bending or extension (for example, a hard material such as tungsten) when puncturing toward a deep part of the brain. The stimulation probe 30 is not particularly limited in diameter, and can have a diameter of, for example, approximately 1 μm to 1 mm or 1 mm to 2.5 mm. The stimulation probe 30 has one or more (for example, two, three, or four) stimulation electrodes 40 distally. The stimulation electrode 40 can have a length of approximately 1 to 5 mm in the long axis direction of the stimulation probe 30. When the stimulation probe 30 has a plurality of stimulation electrodes 40, the stimulation electrodes 40 are not particularly limited in interval, and can be disposed at intervals of, for example, approximately 1 mm to 1.5 mm. Each of the stimulation electrodes 40 may be controlled collectively by one control signal, or each can preferably be separately controlled by an individual control signal. By separately controlling each by an individual control signal to selectively generate a voltage at an electrode that is best suited in relation to the insertion position of the electrode, the brain can be stimulated.

The apparatus 1 of the present invention induces a hibernation-like state in a subject, and does not need to be portable. Here, portable means moving together with the movement of a subject relative to a footing (for example, the ground, or when the subject is in a vehicle, the floor of the vehicle) where the subject is located. Therefore, the apparatus of the present invention can be fixed to an installation site. The apparatus of the present invention can be connected to a power supply, and thus, for example, the apparatus can have no battery or no rechargeable battery. The apparatus 1 of the present invention induces a hibernation-like state in a subject having a disease, and does not need to be portable, but the apparatus is preferably portable from the viewpoint of treating the disease on site when the disease is an acute disease. The apparatus of the present invention can be connected to a power supply, thus for example, the apparatus can have no battery or no rechargeable battery, and from the viewpoint of portability, the apparatus may have a battery or a rechargeable battery so that the apparatus can operate without being connected to a power supply.

### Second Embodiment

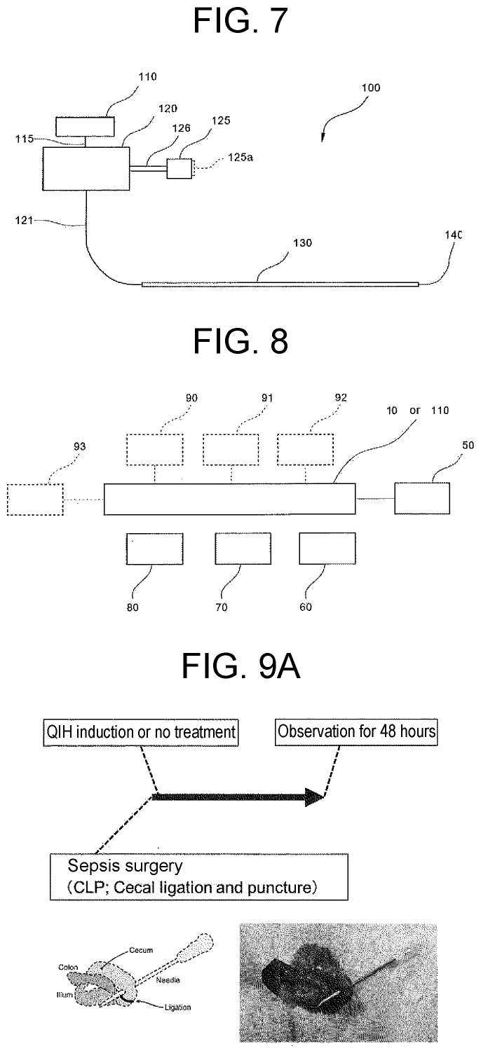

In the first embodiment, an apparatus that electrically stimulates a deep part of the brain was disclosed, and a second embodiment relates to an apparatus that chemically stimulates a deep part of the brain. Hereinafter, the second embodiment will be described with reference to FIG. 7.

In the second embodiment, an apparatus 100 of the present invention includes:
a control unit 110 that transmits a control signal controlling the release of a QRFP-producing neuron-stimulating compound;
a storage unit 125 for the compound;
a compound export unit 120 that receives the control signal from the control unit to export the compound from the storage unit 125 for the compound; and a guide 130 comprising a compound release port 140 and a channel for the compound to the release port 140 and delivering the compound to a QRFP-producing neuron. In the apparatus 100 of the present invention, the control unit 110 is electrically connected to the compound export unit 120 through a wiring 115. The compound export unit 120 receives a control signal from the control unit 110, and according to the control signal thereof, releases the compound accumulated in the storage unit 125 from the storage unit 125 through a channel 126 and a channel 121 and the guide 130 into the brain through the compound release port 140. The compound may be in the form of a solution in which the compound is dissolved in a solvent, and can be fed to the compound release port 140 by a feed mechanism using the compound export unit 120. The storage unit 125 for the compound may have a compound introduction port 125a through which the compound is introduced from the outside. The compound introduction port 125a can supply the compound into a compound storage unit. The compound storage unit 125 may be exposed outside the body. However, when the compound storage unit 125 is exposed outside the body, the compound storage unit 125 is maintained under a sterile condition. The control unit 110 transmits, to the compound export unit 120, a control signal that causes, for example, feeding of 1 μL to 100 μL of the compound per instance of export of the compound.

The guide 130 can be inserted into the brain through stereotaxic surgery in order to allow the compound release port 140 to accurately reach a QRFP-producing neuron. Stereotaxic surgery is a surgery in which the head is fixed with a measurement frame, and an electrode is inserted with a precision of 1 mm or less into a position at which the electrode is to be inserted, the position being determined by a CT scan or MRI. From the viewpoint of stereotaxic surgery, the guide 130 is formed from a material that is hard enough not to cause bending or extension (for example, a hard material such as tungsten) when puncturing toward a deep part of the brain. The stimulation probe 30 can have a diameter of, for example, approximately 1 mm to 2.5 mm.

The apparatus 100 of the present invention induces a hibernation-like state in a subject, and does not need to be portable. Here, portable means moving together with the movement of a subject relative to a footing (for example, the ground, or when the subject is in a vehicle, the floor of the vehicle) where the subject is located. Therefore, the apparatus of the present invention can be fixed to an installation site (for example, the bed on which a subject lies or the floor on which the bed is disposed). The apparatus of the present invention can be connected to a power supply, and thus, for example, the apparatus can have no battery or rechargeable battery. The apparatus 100 of the present invention induces a hibernation-like state in a subject having a disease, and does not need to be portable, but the apparatus is preferably portable from the viewpoint of treating the disease on site when the disease is an acute disease. The apparatus of the present invention can be connected to a power supply, thus for example, the apparatus can have no battery or rechargeable battery, and from the viewpoint of portability, the apparatus may have a battery or a rechargeable battery so that the apparatus can operate without being connected to a power supply.

### (Additional Configurations)

The apparatus 1 of the first embodiment and the apparatus 100 of the second embodiment can further include:
configuration (B):
an outside air temperature gauge 50;
a thermometer 60;
a computation unit 70 that calculates an oxygen consumption of a subject from an oxygen concentration difference between exhaled air and inhaled air from the subject; and
a recording unit 80 that records a measured outside air temperature and at least one numerical value selected from the group consisting of a body temperature and the oxygen consumption {here, the thermometer can preferably be a deep body thermometer that measures a deep body temperature of the subject}. As shown in FIG. 8, (B) above may be included, for example, in the control unit 10 or the control unit 110 (here, although illustration is omitted in FIG. 8, the control units 10 and 110 are connected, by wire or wirelessly, to the voltage generation unit 20 as described in the first embodiment and the second embodiment, respectively). When inducing a hibernation-like state in a subject, the outside air temperature (or ambient temperature of the subject) (T_{A}) is decreased, and also the deep body temperature (T_{B}) and metabolism are decreased. Therefore, by including an outside air temperature gauge that measures the outside air temperature (or ambient temperature of the subject) and a body thermometer (preferably deep body thermometer), the apparatus of the present invention can monitor the relationship between the body temperature (preferably deep body temperature) of the subject and the outside air temperature.

In addition to configuration (B), the apparatus 1 of the first embodiment and the apparatus 100 of the second embodiment may further include, as configuration (C):
a computation unit 205 that calculates the oxygen consumption of the subject from the oxygen concentration difference between the exhaled air and the inhaled air from the subject;
a hypometabolic state (preferably a hibernation-like state) possibility determination unit 206 that determines that the subject has entered a hypometabolic state (preferably a hibernation-like state) or may have entered a hypometabolic state (preferably a hibernation-like state) at least based on a decrease in oxygen consumption of the subject compared with an oxygen consumption of the subject before entering the hypometabolic state (preferably a hibernation-like state); and/or
a death possibility determination unit 207 that determines that the subject is dead or may be dead at least based on the oxygen consumption, a carbon dioxide concentration of the exhaled air of the subject, an electrocardiogram of the subject, or a blood pressure of the subject of zero. The apparatus having configuration (C) above can be used, for example, to determine whether the subject is in a hibernation-like state or may be dead when the subject has a serious disease. The apparatus (C) may further include a determination unit 240 that determines whether the subject is in a hibernating state or in a dead state by inputting the respective determinations from the hypometabolic state (preferably a hibernation-like state) possibility determination unit 206 and the death possibility determination unit 207. The determination unit 240 can be connected to the output device 250, and a result can be output from the output device 250. Therefore, the apparatus (C) may further include an exhaled air measurement unit 201 that measures the oxygen concentration of the exhaled air and an inhaled air measurement unit 202 that measures the oxygen concentration of the inhaled air.

In addition to configuration (B), the apparatus 1 of the first embodiment and the apparatus 100 of the second embodiment may further include:
configuration (C):
a computation unit that calculates an oxygen consumption of a subject from an oxygen concentration difference between exhaled air and inhaled air from the subject;
a hibernation possibility determination unit that determines that the subject has entered a hibernation-like state or may have entered a hibernation-like state at least based on a decrease in both oxygen consumption and theoretical setpoint temperature compared with an oxygen consumption and a theoretical setpoint temperature of the subject in a non-hibernation-like state; and/or
a death possibility determination unit that determines that the subject is dead or may be dead at least based on the oxygen consumption, a carbon dioxide concentration of the exhaled air of the subject, an electrocardiogramaform of the subject, or a blood pressure of the subject of zero. The apparatus having configuration (C) above can be used, for example, to determine whether the subject is in a hibernation-like state or may be dead when the subject has a serious disease. The apparatus having configuration (C) above may further include a deep body thermometer and a body temperature storage unit that stores a body temperature measured by the thermometer. The apparatus having configuration (C) above may further include a computation unit that calculates the theoretical setpoint temperature. The apparatus having configuration (C) above may further include a computation unit that calculates a feedback gain (H) of heat generation. The apparatus having configuration (C) above may further include a deep body thermometer, a body temperature storage unit that stores a body temperature measured by the thermometer, a computation unit that calculates the theoretical set temperature, and a computation unit that calculates a feedback gain (H) of heat generation. The hibernation possibility determination unit may determine that the subject has entered a hibernation-like state or may have entered a hibernation-like state at least based on a decrease in all of oxygen consumption, theoretical setpoint temperature, and feedback gain of heat generation, compared with an oxygen consumption, a theoretical setpoint temperature, and a feedback gain of heat generation of the subject in a non-hibernation-like state.

In addition, the apparatus of the present invention can estimate the oxygen consumption (VO₂) by the subject and can estimate the metabolic state of the subject from the oxygen consumption (VO₂), by including an exhaled air analysis unit 70 that measures an oxygen concentration of the exhaled gas.

In addition, from the deep body temperature (T_{B}) and the oxygen consumption (VO₂), it is also possible to estimate the theoretical setpoint temperature (T_{R}) Of body temperature and the feedback gain (H) of heat generation. The theoretical setpoint temperature (T_{R}) of body temperature is determined as an estimated value of a deep body temperature (T_{B}) when the relationship between the deep body temperature (T_{B}) and an oxygen consumption (VO₂) is determined while varying (for example, decreasing) the outside air temperature (or ambient temperature of the subject) (T_{A}) and the oxygen consumption (VO₂) is zero. The relationship between the deep body temperature (T_{B}) and the oxygen consumption (VO₂) can be determined, for example, by linear regression. In addition, the feedback gain (H) of heat generation can be determined as H=ΔVO₂/ΔT_{B}. Here, ΔVO₂ is the change in VO₂ and ΔT_{B} is the change in T_{B}.

The apparatus of the present invention can further include a recording unit 80 that records a measured outside air temperature and at least one numerical value selected from the group consisting of the body temperature (preferably, deep body temperature) and the oxygen concentration. The apparatus of the present invention can further include an oxygen consumption determination unit 90 that determines the oxygen consumption of the subject from the oxygen concentration of the exhaled gas. The apparatus of the present invention can further include an estimation unit 91 that estimates the theoretical setpoint temperature (T_{R}) of body temperature and the feedback gain (H) of heat generation. The apparatus of the present invention can further include a determination unit 92 that determines whether or not a hibernation-like state has been induced in the subject at least based on the theoretical setpoint temperature (T_{R}) of body temperature and the feedback gain (H) of heat generation, and/or whether or not the subject is dead at least based on an oxygen consumption of zero. The hibernation possibility determination unit and the death possibility determination unit described above can be the same, and may be separately provided in the apparatus as separate units. The apparatus of the present invention can further include an output unit 93 that outputs information on whether or not a hibernation-like state has been induced and/or whether or not the subject is dead. Examples of the output unit 93 include a display that displays the information and/or a printer that prints the information. Examples of the information on whether or not a hibernation-like state has been induced include information indicating that a hibernation-like state has been induced, and information indicating that a hibernation-like state has not been induced, and the information can be output by the output unit 93. Examples of the information on whether or not the subject is dead includes information indicating that the subject is dead and information indicating that the subject is not dead, and the information can be output by the output unit 93.

### Third Embodiment

According to the invention,
provided is an apparatus that determines hibernation, the apparatus including:
a recording unit that records an oxygen consumption (VO₂) and a deep body temperature (T_{B}) recorded under each of at least two different surrounding environment temperature (T_{A}) conditions each of before administration and after administration of a test compound, in a mammalian animal such as a human in whom the test compound has been administered to regions of an anteroventral periventricular nucleus (AVPe), a medial preoptic area (MPA), and a periventricular nucleus (Pe); and
a computation unit that estimates a correlation between the oxygen consumption and the deep body temperature each of before the administration and after the administration, and determines whether or not an extent of a decrease in oxygen consumption when the deep body temperature decreases after the administration compared with before the administration, and determines whether or not an estimated value of the deep body temperature when the oxygen consumption is assumed to be 0 decreases after the administration compared with before the administration, from the estimated correlation; and
including:
a determination unit that determines that the mammalian animal has hibernated at least based on a decrease in the extent of a decrease in oxygen consumption when the deep body temperature decreases, after the administration compared with before the administration, and a decrease in the estimated value of the deep body temperature when the oxygen consumption is assumed to be 0, after the administration compared with before the administration. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

The recording unit records the oxygen consumption (VO₂) and the deep body temperature (T_{B}) recorded under at least two different surrounding environment temperature (T_{A}) conditions. The recording unit stores one VO₂ and one T_{B} in association with one T_{A}. The oxygen consumption (VO₂) and the deep body temperature (T_{B}) recorded are read from the recording unit and then transmitted to the computation unit, and the correlation between the oxygen consumption and the deep body temperature is estimated in the computation unit. In an embodiment, the correlation is linear. After the correlation has been estimated, the computation unit determines whether or not the extent of a decrease in oxygen consumption when the deep body temperature decreases after the administration compared with before the administration, and determines whether or not the estimated value of the deep body temperature when the oxygen consumption is assumed to be 0 decreases after the administration compared with before the administration. Based on the determination by the computation unit, the determination unit can determine that the mammalian animal has hibernated when the extent of a decrease in oxygen consumption when the deep body temperature decreases after the administration compared with before the administration, and the estimated value of the deep body temperature when the oxygen consumption is assumed to be 0 decreases after the administration compared with before the administration. The determination unit can be configured to not determine that the mammalian animal has hibernated (or can determine that the mammalian animal has not hibernated) when the extent of a decrease in oxygen consumption when the deep body temperature decreases does not decrease after the administration compared with before the administration, or the estimated value of the deep body temperature when the oxygen consumption is assumed to be 0 does not decrease after administration compared with before the administration.

The apparatus that determines hibernation according to the third embodiment of the present invention may further include a deep body thermometer and an exhaled gas analysis unit that measures an oxygen concentration of exhaled gas. The apparatus according to the third embodiment may further include an output unit that receives information on determination relating to hibernation from the determination unit, and outputs the information. The information output unit can be a user interface such as a display, can be a recording device that records on a non-volatile memory such as a USB memory or an SD card, can be an information transmission device for wireless communication to the outside, or can be a printing device such as a printer for printing onto a medium such paper.

The apparatus of the first embodiment or the second embodiment may further include the apparatus that determines hibernation according to the third embodiment.

### Stimulation Method of the Present Invention

According to the present invention, a method for decreasing a theoretical setpoint temperature of body temperature and/or a feedback gain of heat generation in a subject is provided. According to the present invention, a method for inducing a hibernation-like state in a subject is provided. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

According to the method of the present invention, the method includes providing an excitatory stimulus to a pyroglutamylated RFamide peptide (QRFP)-producing neuron in the subject. According to the present invention, a method for decreasing a feedback gain of heat generation in a subject, the method including providing an excitatory stimulus to a pyroglutamylated RFamide peptide (QRFP)-producing neuron is also provided. According to the present invention, a method for decreasing a theoretical setpoint temperature of body temperature and a feedback gain of heat generation in a subject, the method including providing an excitatory stimulus to a pyroglutamylated RFamide peptide (QRFP)-producing neuron is also provided. According to the present invention, a method for inducing a hibernation-like state in a subject, the method including providing an excitatory stimulus to a pyroglutamylated RFamide peptide (QRFP)-producing neuron by using a drug or the like is also provided. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

In the method of the present invention, a pyroglutamylated RFamide peptide (QRFP)-producing neuron can be stimulated by using, for example, the apparatus of the present invention. In the method of the present invention, a QRFP-producing neuron can be loaded with a voltage, thereby stimulating the QRFP-producing neuron. In the method of the present invention, a stimulus can be applied to the QRFP-producing neuron by expressing a receptor (for example, hM3Dq) in a fashion specific to a pyroglutamylated RF amide peptide (QRFP)-producing neuron, for example, by using a DREADD method, and administering a ligand (for example, clozapine-N-oxide (CNO)) for the receptor. hM3Dq can be expressed in a QRFP-producing neuron by infecting the QRFP-producing neuron of the subject with a virus (for example, adenovirus or adeno-associated virus) having a gene encoding hM3Dq, which is operably linked to a QRFP promoter. CNO can be administered to the brain, for example, by the apparatus of the invention. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

In the method of the present invention, a pyroglutamylated RFamide peptide (QRFP)-producing neuron can also be stimulated by using, for example, an activating agent for the neuron, in a subject. The activating agent can be screened for by using a QRFP neuron or can be searched for by using a cultured cell in which a receptor that expresses in a QRFP neuron has been forcibly expressed. The activating agent for the neuron may be administered locally to a QRFP-producing neuron by using an applicator. The QRFP-producing neuron-specific activating agent may be administered by intracerebroventricular administration, intrathecal administration, or systemic administration such as intravenous administration. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

The method of the present invention may further include decreasing the outside air temperature. Thereby, T_{B} of the subject can be decreased. It is thought that when T_{B} decreases in a hibernation-like state, a hypometabolic state is created, energy consumption is decreased, and life can be maintained.

The method of the present invention can further include measuring the deep body temperature (T_{B}) of the subject. The method of the present invention can further include measuring the oxygen concentration of exhaled air of the subject.

The method of the present invention can further include estimating the oxygen consumption (VO₂) of the subject. The oxygen consumption (VO₂) of the subject can be estimated, for example, from the difference in oxygen concentration between inhaled air and exhaled air.

The method of the present invention can be a method including:
providing (or recording) an oxygen consumption (VO₂) and a deep body temperature recorded under each of at least two different surrounding environment temperature conditions each of before administration and after administration of a test compound, in a mammalian animal such as a human in whom the test compound has been administered to regions of an anteroventral periventricular nucleus (AVPe), a medial preoptic area (MPA), and a periventricular nucleus (Pe);
estimating a correlation between the oxygen consumption and the deep body temperature each of before the administration and after the administration; and
determining whether or not an extent of a decrease in oxygen consumption when the deep body temperature decreases after the administration compared with before the administration, and determining whether an estimated value of the deep body temperature when the oxygen consumption is assumed to be 0 decreases after the administration compared with before the administration, from the estimated correlation,
wherein a decrease in the extent of a decrease in oxygen consumption when the deep body temperature decreases, after the administration compared with before the administration, and a decrease in the estimated value of the deep body temperature when the oxygen consumption is assumed to be 0, after the administration compared with before the administration indicate that the mammalian animal has hibernated. In a preferable embodiment, the mammalian animal has a disease. In a preferable embodiment, the mammalian animal has a chronic disease. In a preferable embodiment, the mammalian animal has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

In this embodiment, the method of the present invention can further include estimating the theoretical setpoint temperature (T_{R}) of the body temperature of the subject. The theoretical setpoint temperature (T_{R}) is determined as an estimated value of a deep body temperature (T_{B}) when the relationship between the deep body temperature (T_{B}) and an oxygen consumption (VO₂) is determined while varying (for example, decreasing) the outside air temperature (or ambient temperature of the subject) (T_{A}) and the oxygen consumption (VO₂) is zero. The relationship between the deep body temperature (T_{B}) and the oxygen consumption (VO₂) can be determined, for example, by linear regression.

The method of the present invention can further include estimating the feedback gain (H) of heat generation of the subject. The feedback gain (H) of heat generation can be determined as H=ΔVO₂/ΔT_{B}.

The method of the present invention can further include determining whether or not the subject is in a hibernation-like state. Whether or not the subject is in a hibernation-like state can be determined based on whether or not both the theoretical setpoint temperature (T_{R}) of body temperature and the feedback gain (H) of heat generation decrease when the outside air temperature is decreased. If the theoretical setpoint temperature (T_{R}) of body temperature and the feedback gain of heat generation (H) both decrease when the outside air temperature is decreased, it can be determined that the subject is in a hibernation-like state.

The hibernation-like state can be beneficial in improving a life protection function by decreasing the metabolism of the living body. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

### Screening System of the Present Invention

According to the present invention, provided is a method for screening for a substance that provides an excitatory stimulus to a pyroglutamylated RFamide peptide (QRFP)-producing neuron present in regions of an anteroventral periventricular nucleus (AVPe), a medial preoptic area (MPA), and a periventricular nucleus (Pe), the method including:
contacting a test compound with the QRFP-producing neuron isolated;
measuring excitation of the QRFP-producing neuron; and
selecting a test compound that provides an excitatory stimulus to the QRFP-producing neuron. The method can be an in vitro method. The screening can be carried out in a subject. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. In a preferable embodiment, the subject is a disease model. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

The excitation of the QRFP-producing neuron can be measured electrically. The electrical measurement of the excitation of the neuron can be measured, for example, with the depolarization of a membrane potential as an index by an electrophysiological technique by using a conventional method. The membrane potential can be measured, for example, by a neural recording method such as a microelectrode method or by a patch clamp method, or may be measured by using a fluorescent probe for membrane potential measurement. The fluorescent probe for membrane potential measurement is not particularly limited, and examples thereof include 4-(4-(didecylamino) styryl)-N-methylpyridinium iodide (4-Di-10-ASP), bis-(1,3-dibutylbarbituric acid) trimethine oxonol (DiSBAC2(3)), 3,3′-dipropylthiadicarbocyanine iodide (DiSC3(5)), 5,5′,6,6′-tetrachloro-1,1′,3,3′-tetraethylbenzimidazolylcarbocyanine iodide (JC-1), and rhodamine 123. In addition, the excitation of the neuron can also be measured chemically. When the neuron is excited, the intracellular calcium concentration increases. For example, the excitation of the neuron can be measured by using a calcium concentration indicator. As the calcium concentration indicator, various probes such as 1-[6-amino-2-(5-carboxy-2-oxazolyl)-5-benzofuranyloxy]-2-(2-amino-5-methylphenoxy) ethane-N,N,N′,N′-tetraacetic acid and pentaacetoxymethyl ester (Fura 2-AM) are known and can be used in the present invention.

The QRFP-producing neuron is a neuron that is present in the regions of the anteroventral periventricular nucleus (AVPe), the medial preoptic area (MPA), and the periventricular nucleus (Pe), and can be an established cell line of a neuron. As the established cell line of neuron, a line obtained by selecting a line in which the neuron produces QRFP can be used. Whether or not the neuron produces QRFP can be confirmed by a conventional method by using an antibody against QRFP.

### Method for Determining Hibernation According to the Present Invention

The method for determining hibernation according to the present invention analyzes the effect of a drug that induces hibernation, a drug that is expected to induce hibernation, or a drug that has the potential to induce hibernation in a subject. When the subject has entered a hibernation-like state, the hibernation-like state can be maintained or canceled. When the subject does not enter a hibernating state, a further treatment can be provided or the treatment can be discontinued. In a preferable embodiment, the subject has a disease. In a preferable embodiment, the subject has a chronic disease. In a preferable embodiment, the subject has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

The method for determining hibernation according to the present invention can be a computational method. The method for determining hibernation according to the present invention can include no medical intervention. The method for determining hibernation according to the present invention can, for example, make a determination based on input of a measured value without including the measurement itself. The determination result can be basic information for diagnosis by a health care practitioner including a physician.

It may not be easy to quickly distinguish between a hibernation-like state and a dead state based on appearance alone. In an embodiment, the method for determining hibernation according to the present invention can be used to distinguish between hibernation and death. When the subject is in a dead state, a health care practitioner or the like can quickly promote resuscitation and exit from a hibernation-like state.

The method for determining hibernation according to the present invention can be a method for determining (testing, predicting, estimating, computationally determining) whether a test compound induces hibernation or may induce hibernation in a mammalian animal such as a human, the method comprising:
providing (or recording), an oxygen consumption and a deep body temperature recorded under each of at least two different surrounding environment temperature conditions each before administration and after administration of the test compound, in a mammalian animal such as a human in whom the test compound has been administered to regions of an anteroventral periventricular nucleus (AVPe), a medial preoptic area (MPA), and a periventricular nucleus (Pe);
estimating a correlation between the oxygen consumption and the deep body temperature each of before the administration and after the administration; and
determining whether or not an extent of a decrease in oxygen consumption when the deep body temperature decreases after the administration compared with before the administration, and determining whether or not an estimated value of the deep body temperature when the oxygen consumption is assumed to be 0 decreases after the administration compared with before the administration, from the estimated correlation,
wherein a decrease in the extent of a decrease in oxygen consumption when the deep body temperature decreases, after the administration compared with before the administration, and a decrease in the estimated value of the deep body temperature when the oxygen consumption is assumed to be 0, after the administration compared with before the administration indicate that the mammalian animal has hibernated. The mammalian animal can be a non-human mammalian animal. In a preferable embodiment, the mammalian animal has a disease. In a preferable embodiment, the mammalian animal has a chronic disease. In a preferable embodiment, the mammalian animal has an acute disease. In a preferable embodiment, the disease, the chronic disease, or the acute disease is a serious disease. The acute disease can be, for example, one or more selected from the group consisting of sepsis, acute renal failure, acute pneumonia, myocardial infarction, cerebral infarction, cerebral hemorrhage, and hemorrhagic shock. The chronic disease can be one or more selected from the group consisting of a tumor, an autoimmune disease, and aging.

The different surrounding environment temperature conditions can be set by using a temperature control unit (for example, the apparatus of the first embodiment or the second embodiment). The oxygen consumption and the deep body temperature can be determined by using an exhaled gas analysis device and a deep body thermometer, respectively. As the exhaled gas analysis device and the deep body thermometer, ones included in the apparatus of the first embodiment or the second embodiment can be used.

### (Treatment of Disease by Hypometabolic Therapy (Preferably Hibernation Induction))

According to the present invention, provided is a method for treating a subject having a disease or a disease, the method including inducing a hypometabolic state (preferably a hibernation-like state) in the subject. The hypometabolic state can halt or slow the progression of a symptom of any disease. The hypometabolic state can be induced, for example, by hypometabolic therapy. In addition, hibernation puts a subject in a hypothermic and hypometabolic state, and at the same time, can halt or slow the progression of a symptom of any disease. This can slow the progression of a chronic disease or allow ensuring the time until a further treatment in an acute disease, for example, ensuring the time for medical transport. In addition, by inducing a hypometabolic state (preferably a hibernation-like state) to slow progression of the disease, life support of the patient is enabled. Hibernation can be induced in a subject by exciting a QRFP nerve. For example, hibernation can be induced in a subject by the method described above. Hibernation can be induced in a subject by using the apparatus described above. The disease can be, for example, an acute disease. The acute disease can be, for example, one or more selected from the group consisting of sepsis, acute renal failure, acute pneumonia, myocardial infarction, cerebral infarction, cerebral hemorrhage, and hemorrhagic shock. From the viewpoint of promoting the delivery of the administered compound to a QRFP nerve, the acute disease is preferably a disease in which blood circulation is maintained. The disease can be, for example, a chronic disease or condition. The chronic disease can be one or more selected from the group consisting of a tumor, an autoimmune disease, and aging.

### Compound or Candidate Therefor Inducing Hibernation for Treating Disease

According to the present invention, a compound or a candidate therefor that induces hibernation can be obtained by administering a compound to a subject and then testing whether the compound induces hibernation in the subject. Therefore, according to the present invention, provided is a method for selecting a compound or a candidate therefor that induces hibernation, the method including administering a compound to a subject, then testing whether or not the compound induces hibernation in the subject, and selecting a compound that induces hibernation.

According to the invention, also provided is a method for testing a compound or a composition including the compound (for example, pharmaceutical formulation or pharmaceutical composition), the method including administering the compound or the composition to a subject, determining whether or not the compound or the composition induces hibernation in the subject, and obtaining a compound or a composition that induces hibernation. The obtained compound can then be formulated as a pharmaceutical composition. Alternatively, the obtained compound is included in the composition formulated as a pharmaceutical formulation. The subject can be a subject having a disease, particularly a subject having a serious disease, such as a patient having a serious acute disease or chronic disease. Examples of the disease can be as described above. According to the invention, a pharmaceutical composition including the compound thus selected is provided. The pharmaceutical composition can be used to induce hibernation used in the treatment of a disease.

### Apparatus for Treatment of Disease by Hibernation

According to the present invention, an apparatus for the treatment of a disease by hibernation is provided. According to the invention, for example, the apparatus for the treatment of a disease by hibernation can be the apparatus of the first embodiment, the second embodiment, or the third embodiment above. In this embodiment, the apparatus for the treatment of a disease by hibernation may further include a determination unit that estimates or determines that the subject is dead or may be dead at least based on an oxygen consumption of zero (for example, when the oxygen consumption of the subject disappears).

According to the present invention, for example, the apparatus for the treatment of a disease by hibernation is installed in a motor vehicle. According to the present invention, a motor vehicle including the apparatus for the treatment of a disease by hibernation is provided. The motor vehicle can be a vehicle for ambulance transport (for example, ambulance). The motor vehicle can include a bed for laying the patient. The motor vehicle can be able to include a stretcher. The motor vehicle can include a fixture for fixing the stretcher on the bottom of the interior. The fixture can be a vibration isolating trestle. The stretcher can be a roll-in type stretcher. The ambient temperature of the subject can be adjusted to a level comparable to the theoretical setpoint temperature (for example, theoretical setpoint temperature ±5° C., ±4° C., ±2° C., or ±1° C.) by air conditioning or the like. According to the invention, for example, the apparatus for the treatment of a disease by hibernation can be installed in a medical facility or a non-medical facility, preferably a medical facility. In a medical facility, the apparatus therefor can be preferably installed in a high care unit (HCU) and an intensive care unit (ICU). The ambient temperature can be adjusted to a level comparable to the theoretical setpoint temperature (for example, theoretical setpoint temperature ±5° C., ±4° C., ±2° C., or ±1° C.) by air conditioning or the like.

### Hypometabolic State Monitoring Apparatus

In a certain case, such as in the case of a serious disease, it is beneficial to monitor minute oxygen consumption during a hypometabolic state (preferably a hibernation-like state) to confirm patient survival. Monitoring of oxygen consumption can be carried out, for example, by measuring the concentration of oxygen included in each of inhaled air and exhaled air, and comparing the measured value of exhaled air and the measured value of inhaled air. When entering a hypometabolic state (preferably a hibernation-like state), oxygen consumption is lower than before the hypometabolic state (preferably a hibernation-like state) and the difference between the measured values of the oxygen concentrations of exhaled air and inhaled air is smaller, and when the subject dies, the difference between the measured values of the oxygen concentrations of exhaled air and inhaled air disappears. By monitoring the respiration of the patient as described above, the state of the patient (for example, patient having a serious disease or condition, such as patient having a serious acute disease or chronic disease) can be monitored.

According to the present invention, an apparatus that can monitor the hypometabolic state (preferably a hibernation-like state) or the survival state of the patient based on oxygen consumption is provided. According to the present invention, when entering a hypometabolic state (preferably a hibernation-like state), oxygen consumption is lower than before the hypometabolic state (preferably a hibernation-like state) and the difference between the measured values of the oxygen concentrations of exhaled air and inhaled air is smaller, and when the subject dies, the difference between the measured values of the oxygen concentrations of exhaled air and inhaled air disappears. Therefore, according to the present invention, provided is an apparatus that monitors a hypometabolic state (preferably a hibernation-like state) in a subject having a disease in a hypometabolic state (preferably a hibernation-like state), the apparatus including:
a computation unit that calculates an oxygen consumption of the subject from an oxygen concentration difference between exhaled air and inhaled air from the subject;
a hypometabolic state (preferably a hibernation-like state) possibility estimation unit that estimates that the subject has entered a hypometabolic state (preferably a hibernation-like state) or may have entered a hypometabolic state (preferably a hibernation-like state) at least based on a decrease in oxygen consumption compared with an oxygen consumption of the subject before entering the hypometabolic state (preferably a hibernation-like state); and
a death possibility estimation unit that estimates that the subject is dead or may be dead at least based on an oxygen consumption of zero. In addition, according to the present invention, provided is an apparatus that monitors a hibernation-like state in a subject having a disease in a hibernation-like state, the apparatus including:
a computation unit that calculates an oxygen consumption of the subject from an oxygen concentration difference between exhaled air and inhaled air from the subject;
a hibernation possibility estimation unit that estimates that the subject has entered a hibernation-like state or may have entered a hibernation-like state at least based on a decrease in oxygen consumption compared with an oxygen consumption of the subject before entering the hibernation-like state; and
a death possibility estimation unit that estimates that the subject is dead or may be dead at least based on an oxygen consumption of zero. According to the present invention, provided is an apparatus that monitors a hibernation-like state in a subject having a disease in a hibernation-like state, the apparatus including:
a computation unit that calculates an oxygen consumption of the subject from an oxygen concentration difference between exhaled air and inhaled air from the subject;
a hibernation possibility estimation unit that estimates that the subject has entered a hibernation-like state or may have entered a hibernation-like state at least based on a decrease in both oxygen consumption and theoretical setpoint temperature compared with an oxygen consumption and a theoretical setpoint temperature of the subject in a non-hibernation-like state; and
a death possibility estimation unit that estimates that the subject is dead or may be dead at least based on an oxygen consumption of zero.

In addition, according to the present invention, provided is an apparatus that monitors a hypometabolic state (preferably a hibernation-like state) in a subject having a disease in a hypometabolism state (preferably a hibernation-like state), the apparatus including: an exhaled air measurement unit that measures an oxygen concentration of exhaled air of the subject over time; an inhaled air measurement unit that measures an oxygen concentration of inhaled air over time; a computation unit (for example, a processor) that calculates, over time, an oxygen consumption from the oxygen concentration of the inhaled air and the oxygen concentration of the exhaled air at the same time obtained; a memory (for example, a volatile memory or a non-volatile memory) that stores the oxygen consumption calculated over time; and a state determination unit (for example, a processor) that determines that the subject may have entered a hypometabolism state (preferably a hibernation-like state) or may be dead at least based on a change in oxygen consumption over time (preferably also based on a decrease in theoretical setpoint temperature of the subject). In addition, according to the present invention, provided is an apparatus that monitors a hibernation-like state in a subject having a disease in a hibernation-like state, the apparatus including: an exhaled air measurement unit that measures an oxygen concentration of exhaled air of the subject over time; an inhaled air measurement unit that measures an oxygen concentration of inhaled air over time; a computation unit (for example, a processor) that calculates, over time, an oxygen consumption from the oxygen concentration of the inhaled air and the oxygen concentration of the exhaled air at the same time obtained; a memory (for example, a volatile memory or a non-volatile memory) that stores the oxygen consumption calculated over time; and a state determination unit (for example, a processor) that determines that the subject may have entered a hibernating state or may be dead at least based on a change in oxygen consumption over time (preferably also based on a decrease in theoretical setpoint temperature of the subject). The computation unit and the state determination unit may be implemented in the same processor. In an embodiment, the oxygen consumption monitoring apparatus can further include an output unit (for example, a display) that outputs the oxygen consumption or a change therein over time. In an embodiment, the computation unit calculates a graph including a change in oxygen consumption over time, and the output unit outputs the graph. The graph can, for example, have the oxygen consumption on the vertical axis and the time on the horizontal axis. In this embodiment, the output unit may output a graph showing a change in oxygen consumption over time, and the possibility of a hibernating state and/or the possibility of a dead state of the subject. The output unit is, for example, a display, and can display a graph showing a change in oxygen consumption over time and the possibility of a hibernating state of the subject and/or the possibility of a dead state thereof. When the subject may have entered a dead state, a physician or a health care practitioner can examine the subject and if necessary, administer first aid, treatment, or resuscitation.

According to the present invention, an apparatus that monitors a state of a patient by measuring oxygen consumption (the hibernation monitoring apparatus of the present invention) can be incorporated into, coupled to, or caused to cooperate with, the apparatus that determines hibernation according to the third embodiment of the present invention to be operated. According to the present invention, a hibernation monitoring apparatus of the present invention that is incorporated into, or coupled to or caused to cooperate with, the apparatus that determines hibernation according to the third embodiment of the present invention is provided. In an embodiment, according to the present invention, the oxygen consumption monitoring apparatus may further include a reception unit that receives the theoretical setpoint temperature of the subject obtained from the apparatus that determines hibernation according to the third embodiment of the present invention, and may further include a state determination unit (for example, a processor) that determines that the subject may have entered a hibernating state or may be dead at least based on information received in the reception unit and information on the oxygen consumption or the change therein over time. Thereby, it can be possible to improve the determination accuracy of hibernation. In addition, it is possible to more accurately know the state of a patient having an acute stage disease.

According to the present invention, provided are a program that can cause a processor to execute the monitoring, and a method for causing a processor to execute the monitoring, and a method for controlling the apparatus and a program that controls the apparatus, by an apparatus that can monitor a hypometabolic state (preferably a hibernation-like state) or a survival state of a patient based on oxygen consumption. The method for controlling the apparatus that can monitor a hypometabolic state (preferably a hibernation-like state) or a survival state of a patient based on oxygen consumption includes acquiring (for example, in an exhaled air measurement unit) an oxygen concentration of exhaled air of the subject, acquiring (for example, in an inhaled air measurement unit) an oxygen concentration of inhaled air of the subject, and estimating or determining (for example, in a computation unit, for example, a processor) oxygen consumptions of the subject at least at a plurality of time points from the oxygen concentration of the exhaled air and the oxygen concentration of the inhaled air acquired. In this embodiment, the method may further include outputting the oxygen consumption or a change thereof over time. Whether or not the oxygen consumption is lower than normal can be used by a health care practitioner including a physician as information for determining whether the subject is in a hypometabolic state (preferably a hibernation-like state). In addition, in this embodiment, the method may further include determining (for example, in a state determination unit, such as a processor) the possibility that the subject is in a hypometabolic state (preferably a hibernation-like state) and/or the possibility that the subject is dead based on the estimated or determined oxygen consumption, and outputting a result thereof (for example, by a display device, such as a display). A health care practitioner including a physician can use the output result as information for determining whether or not the subject is in a hibernating state and/or whether or not the subject is dead.

According to the present invention, a program for causing the apparatus of the present invention to execute this method is provided.

### Method for Inducing Hypometabolic State in Peripheral Tissue

According to the present invention, a method for inducing a hypometabolic state in a mammalian animal is provided. According to the present invention, a method for inducing a hypometabolic state in a peripheral tissue in a mammalian animal having a disease is provided. According to the present invention, a method for inducing a hypometabolic state in a peripheral tissue in a mammalian animal having a disease, the method including inducing a hypometabolic state in the mammalian animal, thereby inducing a hypometabolic state in a peripheral tissue is provided. In an embodiment, the mammalian animal can be a non-human.

According to the present invention, a method for treating a mammalian animal having a disease (or the disease in the mammalian animal), the method including inducing a hypometabolic state in the mammalian animal is provided. According to the present invention, inducing a hypometabolic state in the mammalian animal can decrease the rate of progression of the disease in the mammalian animal. According to the present invention, inducing a hypometabolic state in the mammalian animal can also prolong survival time (or time to death) in the mammalian animal. According to the present invention, inducing a hypometabolic state in the mammalian animal can also decrease the death probability in the mammalian animal.

According to the present invention, a hypometabolic state can be induced by hypometabolic therapy. According to the invention, the hypometabolic state can be a hibernation-like state. A hibernation-like state can be induced by the method according to the invention. For example, a hibernation-like state can be induced by stimulating a QRFP neuron. The stimulation of a QRFP neuron can be carried out by using the apparatus of the present invention.

A mammalian animal in a hypometabolic state can be followed up. The follow-up can be carried out by monitoring a change in oxygen consumption over time.

According to the invention, the treatment can be emergency care or life-sustaining treatment. According to the present invention, for example, the treatment can be carried out during transport of the animal to a medical facility (for example, hospital) (for example, during ambulance transport) or in an intensive care unit of a medical facility.

According to the present invention, a method for testing effectiveness of a treatment, the method including:
subjecting a mammalian animal having a disease to the treatment;
following up the treated mammalian animal; and
determining any selected from whether or not the treatment decreases a rate of progression of the disease, whether or not the treatment decreases mortality, and whether or not the treatment prolongs survival time compared with a control group not receiving the treatment. This method can further include determining that the treatment is effective based on corresponding to at least one or more selected from decreasing the rate of progression of the disease, decreasing mortality, and prolonging survival time with the treatment compared with a control group not receiving the treatment. The treatment can be, for example, a treatment that induces a hypometabolic state (preferably a hibernation-like state), and in this case, it can be shown that the treatment is effective.

### EXAMPLES

### Experimental Method

### (1) Animals

All animal experiments were carried out at the International Institute for Integrative Sleep Medicine (IIIS) of the University of Tsukuba, and the RIKEN Center for Biosystems Dynamics Research (BDR) according to guidelines for animal experiments. The approval of the animal experiments committee of each institution was obtained, and thus the NIH guidelines were followed. With the exception of the torpor-inducing experiment, mice were given free access to feed and water and maintained at a T_{A} of 22° C. and a relative humidity of 50% on a 12-hour light/12-hour dark cycle. It was found that mice having a body weight of 34 g or more did not exhibit reproducible FIT, and thus heavy mice weighing 34 g or more were excluded from the torpor experiment.

Qrfp-iCre mice were prepared by homologous recombination in C57BL/6N embryonic stem cells and transplantation in 8-cell stage embryos (ICR). A targeting vector was designed to replace the entire coding region of the prepro-Qrfp sequence in exon 2 of the Orfp gene with iCre and a pgk-Neo cassette so that the endogenous Qrfp promoter promoted the expression of iCre. Chimeric mice were crossbred with C57BL/6J females (Jackson Labs). The pgk-Neo cassette was deleted by crossbreeding with FLP66 mice backcrossed to C57BL/6J mice at least ten times. First, F1 hybrids were prepared from heterozygotes crossbred with heterozygotes. These mice were backcrossed to the C57BL/6J mice at least eight times.

Rosa26^{dreaddm3} and Rosa26^{dreaddm4} mice were prepared by homologous recombination in C57BL/6N embryonic stem cells, and after that the same procedure as described above for the Qrfp-iCre mice was followed.

### (2) Virus

As previously described³³, AAV was prepared by using triple transfection and a helper-free method. The final purified virus was stored at −80° C. The titers of recombinant AAV vectors were measured by quantitative PCR. AAV10-EF1α-DIO-TVA-mCherry, 4×10¹³; AAV10-CAG-DIO-RG, 1×10¹³; AAV10-EF1α-DIO-hM3Dq-mCherry, 1.64×10¹²; AAV10-EF1α-DIO-mCherry, 1.44×10¹²; AAV10-EF1α-DIO-SSFO-EYFP, 1.35×10¹²; AAV2/9-hsyn-DIO-TeTxLC-GFP, 6.24×10¹⁴; AAV_{2/9}-hsyn-DIO-GFP, 4×10¹² genome copies/ml. A recombinant rabies vector was prepared by a previously reported method^{22,34}. The titer of SADAG-GFP (EnvA) was 4.2×10⁸ infectious units/ml.

### (3) Surgery

In order to inject the AAV vector, male Orfp-iCre heterozygous mice (8 to 12 weeks old) were anesthetized with isoflurane and placed in a stereotactic frame (David Kopf Instruments).

For chemogenetic manipulation, the Qrfp-iCre mice were injected with AAV10-EF1α-DIO-hM3Dq-mCherry at a rate of 0.1 μm/min by using a Hamilton injection needle into the hypothalamus (for MB injection, anteroposterior direction (AP), −0.46 mm; mediolateral direction (ML), +0.25 mm; dorsoventral direction (DV), −5.75 mm; 0.50 μl at each site; LH injection; AP, −1.00 mm; ML, +1.00 mm; DV, −5.00 mm; 0.30 μl at each site). The needle was left in place for 10 minutes after the injection.

For optogenetic manipulation, AVPe (AP, 0.38 mm; ML, 0.25 mm; DV, −5.50 mm from bregma) was unilaterally injected with AAV10-EF1α-DIO-SSFO-EYFP. After that, optical fibers were implanted bilaterally above AVPe (AP: 0.38 mm, ML: +0.25 mm, DV: −5.20 mm), bilaterally in DMH (AP: − 1.70 mm, ML: +0.25 mm, DV: − 4.75 mm), or unilaterally in RPa (AP: − 6.00 mm, ML: 0.00 mm, DV: − 5.50 mm) (FIG. 2j). After a recovery period of at least 2 weeks in individual cages post-injection, and then the mice were subjected to an infrared thermal imaging experiment. Behavioral data was included only when these viruses were specifically targeted to Q-neurons and the optical fiber implants were accurately disposed.

### (4) Recording Biological Signal

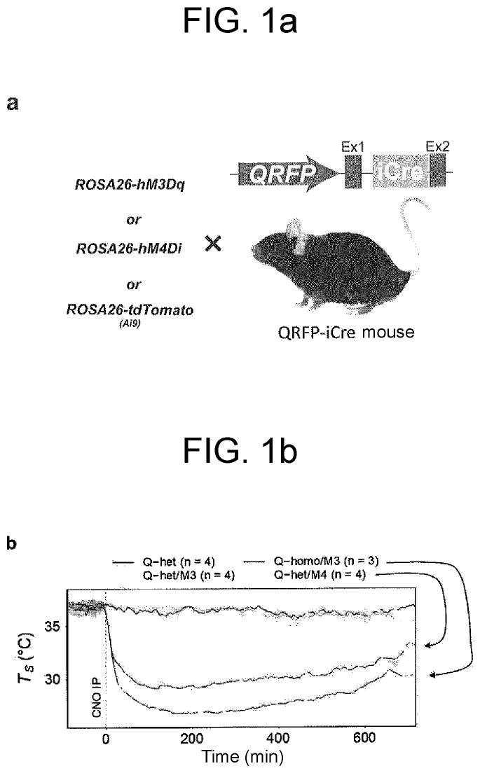

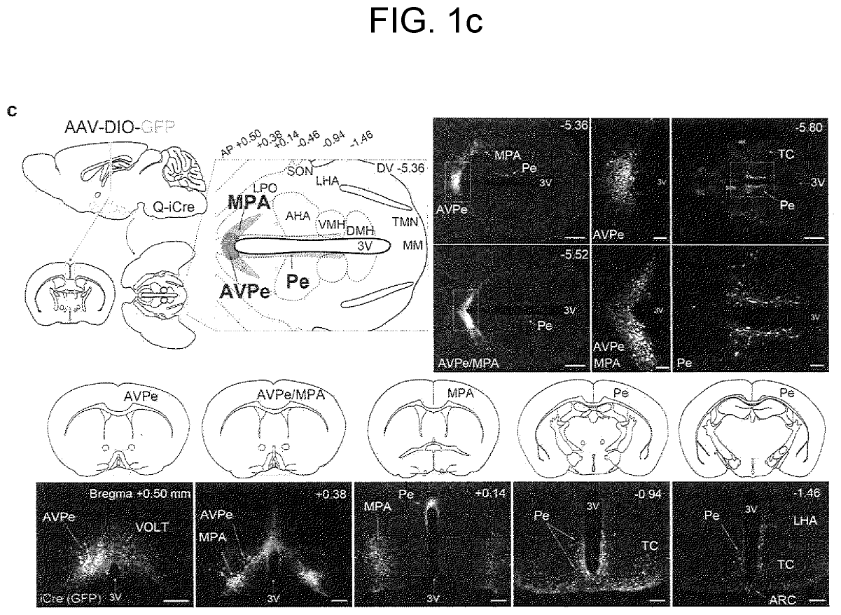

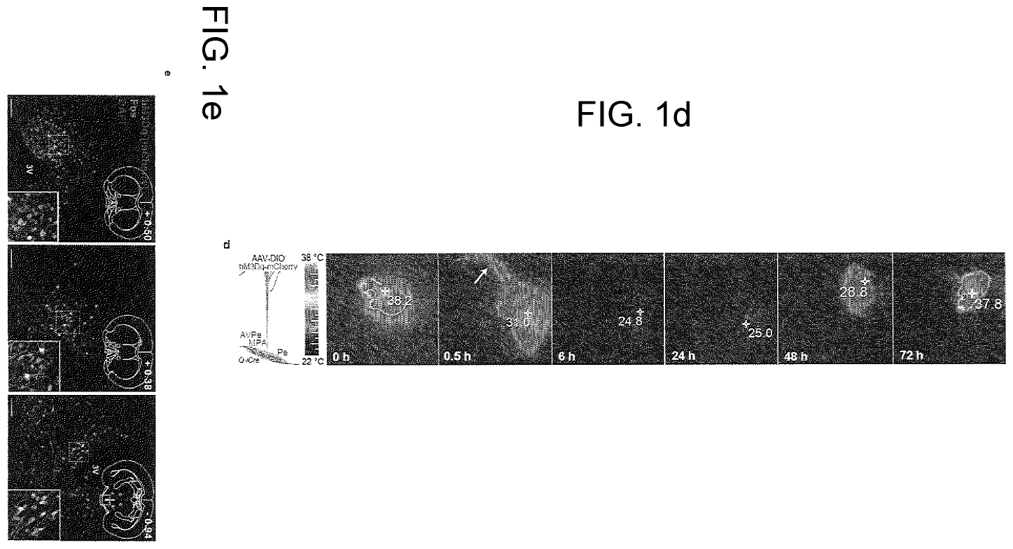

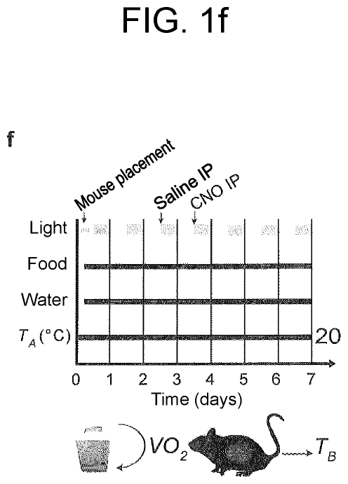

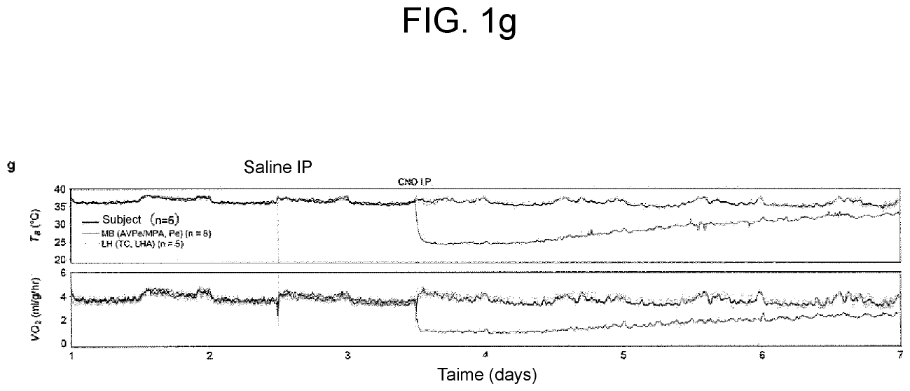

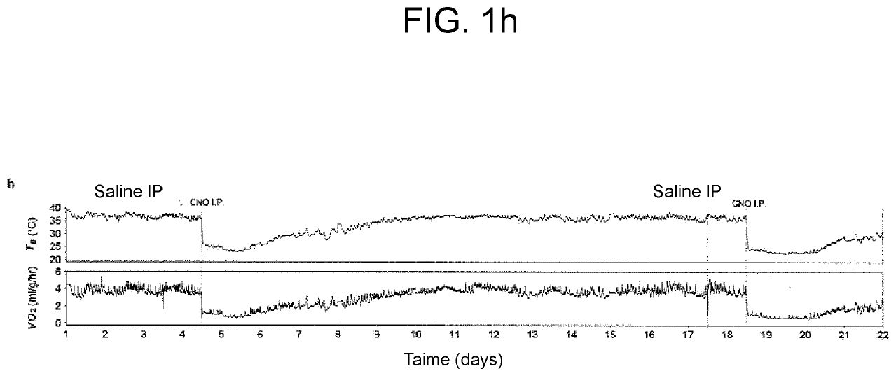

For thermographic analysis, the mice were placed in an experimental cage (25×15×10 cm) and monitored by using an infrared thermal imaging camera (InfReC R500 EX; NIPPON AVIONICS) placed 30 cm above the cage floor. In order to clearly detect the surface temperature, the dorsal hair was removed by using hair clippers one day before the start of the experiment. Thermograms of DREADD and optogenetic experiments were collected at 0.5 Hz and 1 Hz, respectively, and analyzed by using the InfReC Analyzer NS9500 Professional software (NIPPON AVIONICS). The highest temperature in one frame was used as T_{S} of the animal (FIG. 1d).

In order to record deep body temperature, oxygen consumption, EEGs, ECGs, and respiratory patterns, each animal was housed in a temperature-controlled chamber (HC-100, Shin Factory or LP-400P-AR, Nippon Medical & Chemical Instruments Co., Ltd.). In order to continuously record T_{B} (intraperitoneal temperature), a telemetry temperature sensor (TA11TA-F10, DSI) was implanted in the abdominal cavity of the animal under general inhalation anesthesia at least 7 days before recording. VO₂ and the carbon dioxide discharge rate (VCO₂) of the animal were continuously recorded by using a respiratory gas analyzer (ARCO-2000 mass spectrometer, ARCO System). The respiratory coefficient was calculated from the VCO₂/VO₂ ratio.

An EEG and an ECG were recorded by using an implanted remote measurement transmitter (F20-EET or HD-X02, DSI). For EEG recording, two stainless steel screws (1 mm in diameter) were soldered to wires of a telemetry transmitter and inserted through the skull of the cortex (AP, 1.00 mm; right, 1.50 mm from bregma or lambda) under general anesthesia. Two other wires from the transmitter were placed on the surface of the thoracic cavity, and an ECG was recorded. The mice were allowed to recover from surgery for at least 10 days. An EEG/ECG data collection system was composed of a transmitter, an analog-digital converter, and a recording computer with the software Ponemah Physiology Platform (version 6.30, DSI). The sampling rate was 500 Hz for both the EEG and the ECG, and the data was converted into ASCII format for review. The heart rate was evaluated by visual observation of the waveform.

The respiratory flow was recorded by using a non-invasive respiratory flow recording system³⁵. Specifically, mice were placed in a metabolic chamber (TMC-1213-PMMA, Minamiderika Shokai) having an air flow of at least 0.3 L/min. The chamber was connected to a pressure sensor (PMD-8203-3G, Biotex), and the pressure difference between the outside and the inside of the chamber was detected. When the animal is respiring, the outside-to-inside pressure difference increases during inhalation and decreases during exhalation³⁵. An analog signal output from the sensor was converted to a digital signal by an AD converter (NI-9205, National Instruments) at 250 Hz, and stored in a computer by using data logging software developed by Biotex, Inc.

### (5) FIT Induction

An experiment to induce daily torpor was designed to record the metabolism of the animal for at least three days. The animal was introduced into the chamber the day before the start of recording (day 0). Food and water were available ad libitum. T_{A} was set as indicated on day 0 and maintained at a constant level throughout the experiment. The telemetry temperature sensor implanted in the mouse was turned on before the mouse was placed in the chamber. The standard experimental design was as follows. On day 2, the food was removed at ZT-0 in order to induce daily torpor. After 24 hours, the food was returned to each animal at ZT-0 on day 3.

### (6) Recording Metabolism During Drug Administration

The DREADD agonist CNO (clozapine N-oxide, Abcam, ab141704) was dissolved in physiological saline at a dose of 100 μg/mL, and the resulting solution was frozen at −20° C. The CNO solution was thawed on site and then intraperitoneally administered to each mouse at a dose of 1 mg/kg. The adenosinea receptor agonist CHA (N6-cyclohexyladenosine, Sigma-Aldrich, C9901) was dissolved in physiological saline at a concentration of 250 μg/mL, and the resulting solution was intraperitoneally administered to each mouse at a dose of 2.5 mg/kg.

### (7) Recording Metabolism During General Anesthesia

In addition to the above T_{B}, VO₂, and video recording (see “Recording biological signals”), the entrance to the metabolic chamber was directly connected to the exit of an inhalation anesthesia apparatus (NARCOBIT-E, Natsume Seisakusho Co., Ltd.). 1% isoflurane was given for 30 minutes at T_{A}=28° C. and then for 90 minutes at T_{A}=12° C. After the experiment, the animals were warmed on a hot plate, and the recovery was confirmed.

### (8) Immunohistochemical Staining

Each mouse was deeply anesthetized with isoflurane, transcardially perfused with 10% sucrose in water, and subsequently perfused with ice-cooled 4% paraformaldehyde (4% PFA) in a 0.1 M phosphate buffer having a pH of 7.4, and the brain was removed. The brain was left in 4% PFA overnight at 4° ° C., then fixed, incubated overnight at 4° C. in 30% sucrose in a 0.1 M phosphate buffered saline (PBS) having a pH of 7.4, immersed in a Tissuue-Tek O.C.T. compound (Sakura) in a cryomold, and frozen at −80° C. until sectioned. The brain was sliced coronally by using a cryostat (CM1860, Leica) every 50 μm into four equal series, collected in a 6-well plate filled with ice-cooled PBS, and washed three times with PBS at room temperature (RT). Unless otherwise specified, the following incubation step was carried out with gently shaking on an orbital shaker. Brain sections were incubated for 1 hour at room temperature in 1% Triton X-100 in PBS. The sections were blocked at room temperature for 1 hour by using 10% Blocking One (NACALAI TESQUE) in 0.3% Triton X-100-treated PBS (blocking solution) without shaking. The sections were incubated overnight at 4° C. in primary antibodies diluted with a blocking solution (the diluted solution and the type of each antibody are shown below), and then washed three times, incubated overnight at 4° C. along with secondary antibodies, washed with PBS, and then mounted and covered with a cover glass by using HardSet Antifade Mounting Medium (VECTASHIELD) with DAPI.

The primary antibodies used in the present study were rabbit anti-cFos (1:4000, ABE457, Millipore), goat anti-mCherry (1:15000, AB0040-200, SICGEN), rat anti-GFP (1:5000, 04404-84, NACALAI TESQUE), mouse anti-TH (1:1000, sc-25269, Santa Cruz Biotechnology), mouse anti-orexin A (1:200, sc-80263, Santa Cruz Biotechnology), and rabbit anti-MCH (1:2000, M8440, SIGMA). The secondary antibodies are as follows: Alexa Fluor 488 donkey anti-rat, 488 donkey anti-rabbit, 594-donkey anti-rabbit, 594 donkey anti-goat, 647 donkey anti-mouse, and 647 donkey anti-rabbit (1:1000, Invitrogen). For Nissl staining, sections were counterstained with NeuroTrace 435/455 Blue Flue Fluorescent Nissl Stain (1:500, N-21479, Invitrogen) during the secondary antibody step, and covered with a cover glass by using FluorSave Reagent (Millipore). Brain regions were determined by using mouse brain maps from Paxinos and Franklin³⁶.

### (9) In Situ Hybridization

Fluorescence in situ hybridization was carried out by using RNAscope Fluorescent Multiple Kit (Advanced Cell Diagnostics) and by using probes designed for RNAscope Fluorescent Multiplex in situ hybridization (ACDBio RNAscope Probe-Mm-Qrfp #4643411, mCherry #43201, Mm-Slc32a1 #319191, mM-Slc17a6 #319171). The brain was dissected and immediately frozen in 2-methylbutane on dry ice and stored in a frozen embedding medium at −80° C. Prior to the dissection, the brain was cooled to −16° C. in a cryostat for 1 hour. The brain was cut into 20 μm coronal sections by using a cryostat (Leica CM1860UV), and the sections were mounted on Superfrost Plus Microscope Slides (Fisherbrand). Pretreatment methods and RNAscope Fluorescent Multiplex Assay were carried out accurately according to the RNAscope Assay Guide (document numbers 320513 and 320293, respectively).

### (10) Retrograde Tracing of Q-Neurons

Male Qrfp-iCre mice (10 to 12 weeks old) were injected with the following viruses. AAV10-DIO-TVA-mCherry and AAV10-DIO-RG were delivered to express TVA-mCherry and RG in Q-neurons in the MB region (see above for the procedure and the coordinates). After two weeks, SADAG-GFP (EnvA) was injected into the same site. Starter neurons and input (single GFP-positive) neurons were detected in whole brain sections by using each of a Leica TCS SP8 laser confocal microscope and Zeiss Axio Zoom. V16.

(11) Blood chemistry test
Blood was Collected from the Mice Under Anesthesia by a left ventricular puncture by using a 25 gauge needle. The collected blood was stored on ice for not more than 2 hours. The samples were centrifuged at 2000 G for 10 minutes at 4° C., and the supernatants were collected and frozen at −30° C. The frozen serum specimens were sent to FUJIFILM Wako Pure Chemical Corporation, and the Na (mEq/L), K (mEq/L), Cl (mEq/L), AST (IU/L), ALT (IU/L), LDH (IU/L), CK (IU/L), GLU (mg/dL), and total ketone body (umol/L) concentrations were measured.

### (12) Electrophysiological Analysis

The mice were decapitated under deep anesthesia using isoflurane (Pfizer). The brains were extracted and cooled in an ice-cold cutting solution containing the following (mM): 125 mM choline chloride, 25 mM NaHCO₃, 10 mM D(+)-glucose, 7 mM MgCl₂, 2.5 mM KCl, 1.25 mM NaH₂PO₄, and 0.5 mM CaCl₂ bubbled with O₂ (95%) and CO₂ (5%). Horizontal brain slices (250 μm in thickness) including the hypothalamus were prepared by using a vibratome (VT1200S, Leica) and were maintained at room temperature for 1 hour in artificial CSF (ACSF) containing the following (mM): 125 mM NaCl, 26 mM NaHCO₃, 10 mM D(+)-glucose, 2.5 mM KCl, 2 mM CaCl₂), and 1 mM MgSO₄ bubbled with O₂ (95%) and CO₂ (5%). The electrodes (5 to 8 MΩ) were filled with an internal solution containing the following (mM): 125 mM K-gluconate, 10 mM HEPES, 10 mM phosphocreatine, 0.05 mM tolbutamide, 4 mM NaCl, 4 mM ATP, 2 mM MgCl₂, 0.4 mM GTP, and 0.2 mM EGTA, pH 7.3, adjusted with KOH). Firing of hM3Dq-mCherry-expressing neurons was recorded in current-clamp mode at a temperature of 30° C. CNO (1 μM) was bath-applied, and the effect was investigated. The membrane potential and data acquisition were controlled by using a combination of a MultiClamp 700B amplifier, a Digidata 1440A A/D converter, and Clampex 10.3 software (Molecular Devices).

### (13) 3D Imaging of Transparent Mouse Brain

Transparent mouse brains were prepared by the Scales method as previously described³⁷. Scale solutions were prepared by using a urea crystal (Wako Pure Chemical Industries, Ltd., 217-00615), D(−)-sorbitol (Wako Pure Chemical Industries, Ltd., 199-14731), methyl-β-cyclodextrin (Tokyo Chemical Industry Co., Ltd., M1356), γ-cyclodextrin (Wako Pure Chemical Industries, Ltd., 037-10643), N-acetyl-L-hydroxyproline (Skin Essential Actives, Taiwan), dimethyl sulfoxide (DMSO) (Wako Pure Chemical Industries, Ltd., 043-07216), glycerol (Sigma, G9012), and Triton X-100 (Nacalai Tesque, 35501-15). The brains of the Qrfp-iCre mice injected with AAV-DIO-GFP were fixed and made transparent by using ScaleS. Images were then obtained by using a laser confocal microscope (Olympus, XLSLPN25XGMP (NA 1.00, WD: 8 mm) (RI: 1.41 to 1.52)).

### (14) Statistical Analysis

In the present study, Bayesian statistics were applied to evaluate the hypothesis of the inventors and the experimental results. The inventors designed a statistical model having parameters representing the structure of the hypothesis and fit the model to the experimental results. Bayesian inference estimates a posterior probability distribution of model parameters from a likelihood distribution and a prior probability distribution of the parameters. The posterior distribution provides information about how the model can explain the hypothesis from experimental results. Bayesian modeling can explicitly include all types of uncertainty, and thus the Bayesian modeling can handle data with noise in an observation, or can sufficiently utilize information from a small number of samples potentially having a wide range of uncertainty. Further, the Bayesian modeling can handle a plurality of layers of a plurality of groups having different numbers of samples by using hierarchical modeling. These advantages of Bayesian inference are all suitable for addressing problems often encountered in animal experiments. Model fitting was carried out by using Hamiltonian Monte Carlo having a No-U-Turn Sampler, an adaptive variant, as run with version 2.18.0 of Stan having the RStan library³⁸ of version 3.52 of R39. Convergence was evaluated by examining the trace plots:

Gelman−Rubin\hat{R} [Expression 1]
and estimating the number of effective samples. The prior probability density function of the model was defined to be weakly informative and conservative, and was stipulated in the following sections. The basic principles and techniques for designing the statistical models were based on the book Statistical Rethinking⁴⁰. The source code of the models and the data used in the analysis can both be obtained from https://briefcase.riken.jp/public/JjtgwAnqQ81AgyI. (These will be protected with the password “qih” and made public for evaluation.)

The body weights of the Qrfp-iCre mice were modeled by a state-space hierarchical model (code folder QRFP_KO_BW) with a predetermined age and strain. Animals in each group; wild-type (n=9), heterozygous (n=9), and homozygous (n=10) Orfp-iCre mice were raised in their respective cages without identification of individuals. When the unobservable baseline of body weight is defined as the time variable B_{t,s}, where t is the time point, and the strain index (1, 2, and 3 for the wild-type, heterozygous, and homozygous Qrfp-iCre mice, respectively) is expressed by the trend n_{t,s} and the total time point T, the observed state Y_{t,i} can be described by modeling the observation error through a log-normal distribution as follows.

$$\begin{matrix}[ Expression 2 ] & \\ {Y}_{t , s} ∼ Log - normal ( log ( {B}_{t , s} ) - \frac{{( {σ}_{1} )}^{2}}{2} , {σ}_{1} ) & ( 1 ) \\ \begin{matrix}{B}_{1 , s} = + {η}_{1} , t = 1 \\ {B}_{2 , s} - {B}_{1 , s} = {B}_{1 , s} + {η}_{2 , s} , t = 2 \\ {B}_{t , s} - {B}_{t - 1 , s} = {B}_{t - 1 , s} - {B}_{t - 2 , s} + {η}_{t , s} , t \geq 3\end{matrix} \} & ( 2 ) \\ {η}_{t , s} ∼ Normal ( 0 , {σ}_{2} ) & ( 3 ) \\ t = 1 ⋯ T & ( 4 ) \\ s = \{ 1 , 2 , 3 \} & ( 5 )\end{matrix}$$

A uniform prior probability density function was applied to all parameters except σ1 and σ2, which were drawn from standard half-normal distributions.

The spike frequency of Qrfp-positive neurons in brain slices was modeled by parameterizing the difference in spike frequency when neurons were activated by CNO (code folder Patch M3_CNO). When the total number of slices is K, and the observed spike frequencies of the control and the CNO administration recording of the i-th slice are Bᵢ and Cᵢ, respectively, Bᵢ is modeled by β_{BASE} with observation error, and Cᵢ is modeled by the sum of β_{CNO} and β_{BASE} with observation error. The spiking frequency is a positive real number, and thus the error can be modeled by a log-normal distribution, and thus Bᵢ and Cᵢ can be described as follows.

$$\begin{matrix}[ Expression 3 ] & \\ {B}_{i} ∼ Log - normal ( log ( {β}_{BASE} ) - \frac{{( {σ}_{ERROR} )}^{2}}{2} , {σ}_{ERROR} ) & ( 6 ) \\ {C}_{i} ∼ Log - normal ( log ( {β}_{BASE} + {β}_{CNO} ) - \frac{{( {σ}_{ERROR} )}^{2}}{2} , {σ}_{ERROR} ) & ( 7 ) \\ {β}_{BASE} ∼ Normal ( 0 , {σ}_{BASE} ) & ( 8 ) \\ {β}_{CNO} ∼ Normal ( 0 , {σ}_{CNO} ) & ( 9 ) \\ i = 1 ⋯ K & ( 10 )\end{matrix}$$

All σ were sampled from standard half-normal distributions.

T_{S} of light stimulated animals was modeled with a hierarchical multilayer model (FIG. 2l, code folder SSFO_Opto). Four groups of animals were included in this experiment. T_{S} was recorded at 1 Hz, the median value was saved every 10 seconds, and further analysis was carried out. All T_{S} recorded 115 to 125 minutes after the first light stimulation were included in the analysis. When K is the total number of animals and Y is T_{S} during a time of interest for mouse j belonging to i, Y can be expressed as the sum of the global average parameter β, the group parameter β_{GROUP}, and the individual mouse parameter β_{MOUSE}, with observation noise modeled with a Cauchy distribution of the scale parameter σ_{ERROR}.

[Expression 4]

Y_{i,j}˜Cauchy(β+β_{GROUP[i]}+β_{MOUSE[j]},σ_{ERROR}) (11)

β_{GROUP}˜Normal(0,σ_{GROUP}) (12)

β_{MOUSE}˜Normal(0,σ_{MOUSE}) (13)

i={1,2,3,4} (14)

j=1 . . . K (15)

All σ were sampled from standard half-normal distributions. The between-group differences in T_{S} were compared by estimating the average T_{S} of each group from the posterior distribution, which is the sum of β and β_{GROUP} having normally distributed noise with a standard deviation of σ_{MOUSE}.

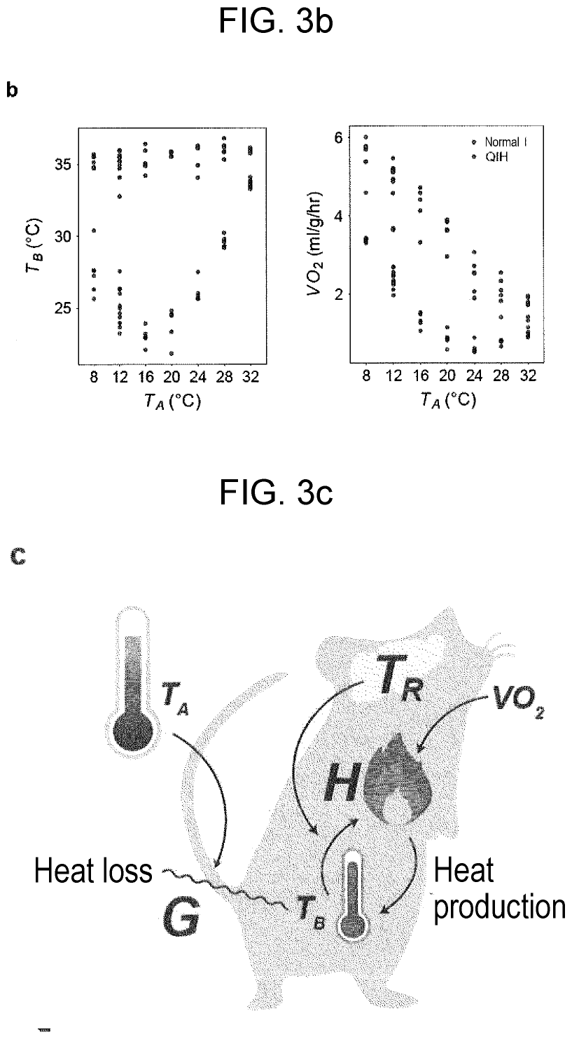

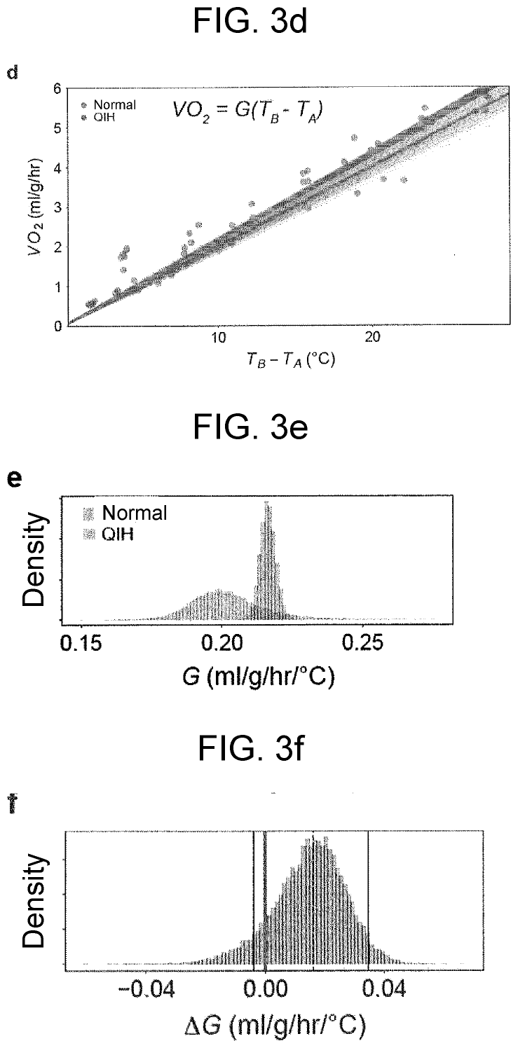

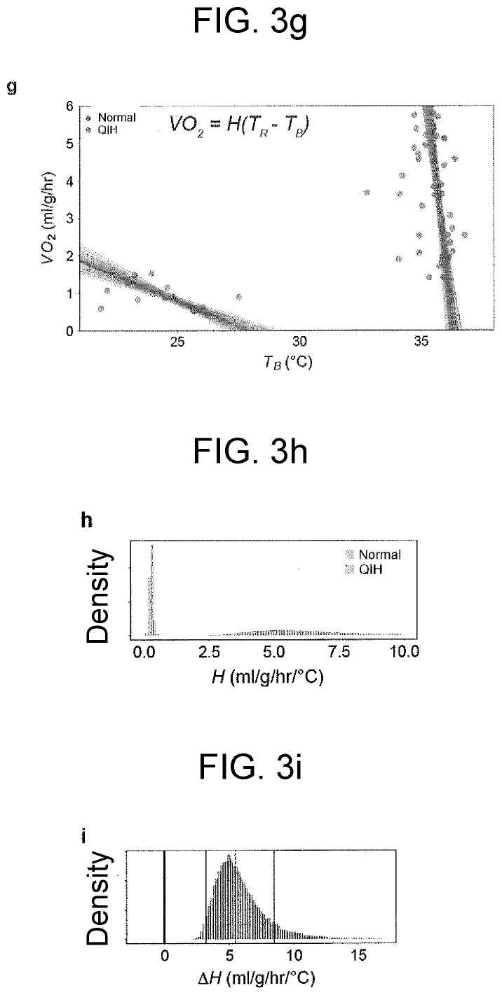

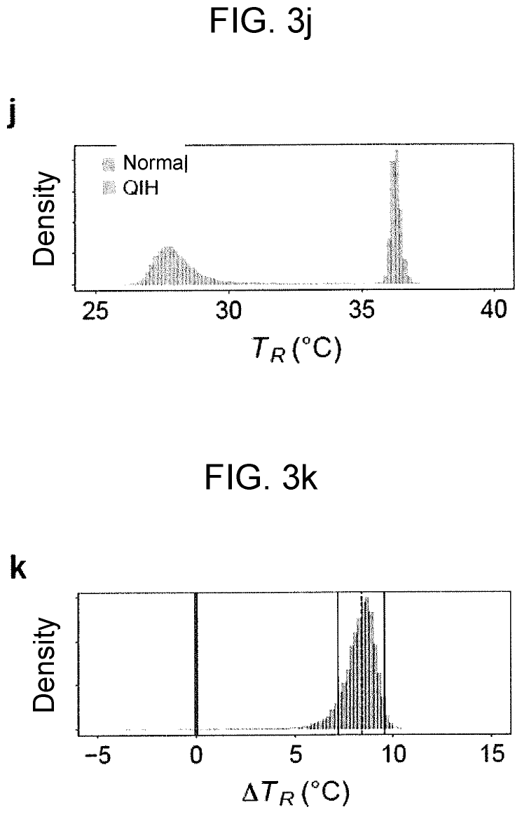

In order to evaluate the thermoregulatory system under QIH and normal conditions, the heat loss and the heat production of the animals were described by a hierarchical multilayer model (FIGS. 3c to 3k, code folder QIH_GTRH). Three parameters of G, T_{R}, and H under two metabolic conditions, that is, normal and QIH, were estimated from a metabolically stable state of the animals at various T_{A}s. The detailed method was previously described³. In brief, a linear model consisting of the controllable parameter T_{A} and the observable parameters T_{B} and VO₂ was fitted to the experimental results for both T_{B} and VO₂ by using T_{A} as a predictor having normally distributed noise. Next, G, T_{R}, and H were estimated by using the posterior distributions of the slope and the intercept coefficient of each model. In this analysis, the prior probability density function of the standard deviation of noise was a standard half-normal distribution, and the other parameters used a positive region of a uniform distribution except for the intercept coefficient of T_{B}, which used a uniform distribution due to negative values.

Circadian changes in metabolism in Q-TeTxLC mice were analyzed by modeling the metabolism by clustering the recorded values into the L-phase and the D-phase (code folder TeTxLC_LD). In particular, when Y is the observed T_{B} of group i in phase j, Y can be expressed as the sum of the basal metabolism (L-phase metabolism) and the difference between the D-phases, and the normally distributed observation noise is as follows.

$$\begin{matrix}[ Expression 5 ] & \\ {Y}_{i , j} ∼ Normal ( {β}_{BASE [ i ]} + {β}_{DARK [ i ]} {P}_{j} , {σ}_{ERROR} ) & ( 16 ) \\ {β}_{BASE} ∼ Normal ( 0 , {σ}_{BASE} ) & ( 17 ) \\ {β}_{DARK} ∼ Normal ( 0 , {σ}_{DARK} ) & ( 18 ) \\ i = \{ 1 : control , 2 : TeTxLC \} & ( 19 ) \\ j = \{ 1 : L - phase , 2 : D - phase \} & ( 20 ) \\ \{ \begin{matrix}{P}_{1} = 0 \\ {P}_{2} = 1\end{matrix} & ( 21 )\end{matrix}$$

All σ were sampled from standard half-normal distributions. In the modeling of VO₂, VO₂ assumes only a positive real number, and thus the basic model structure was the same as that of the T_{B} modeling, except that the observation error was modeled as a log-normal distribution.

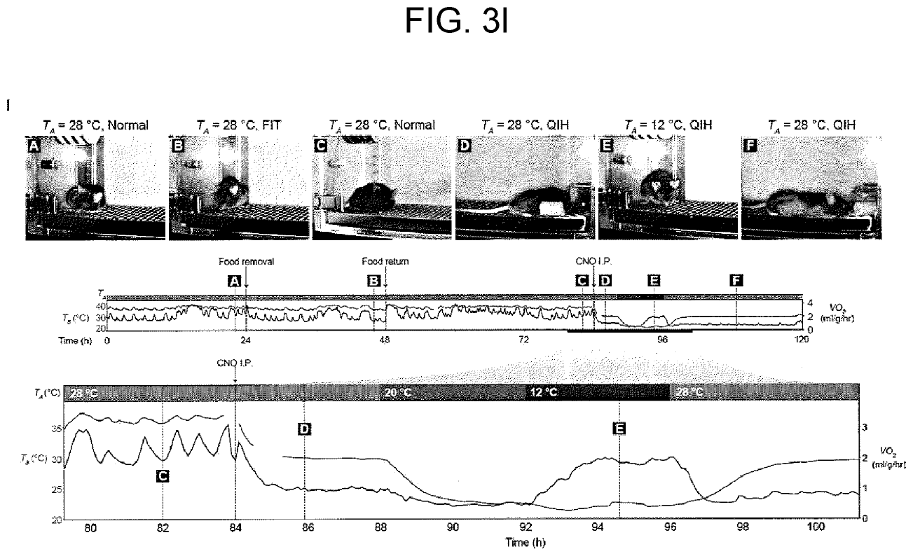

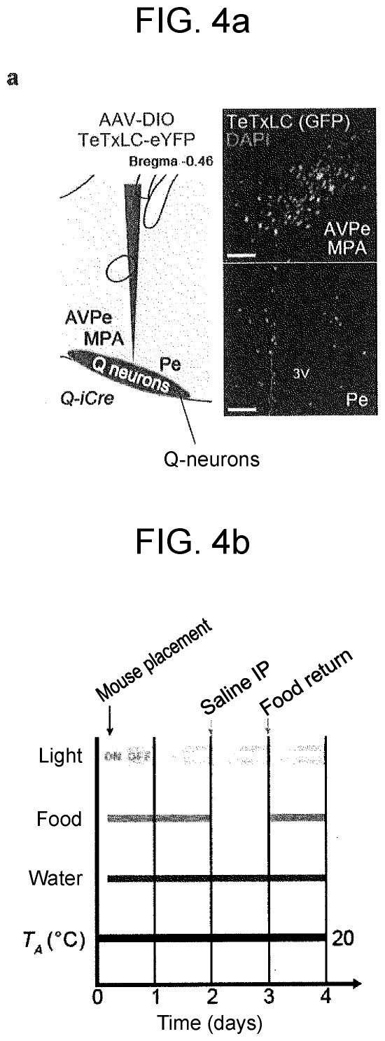

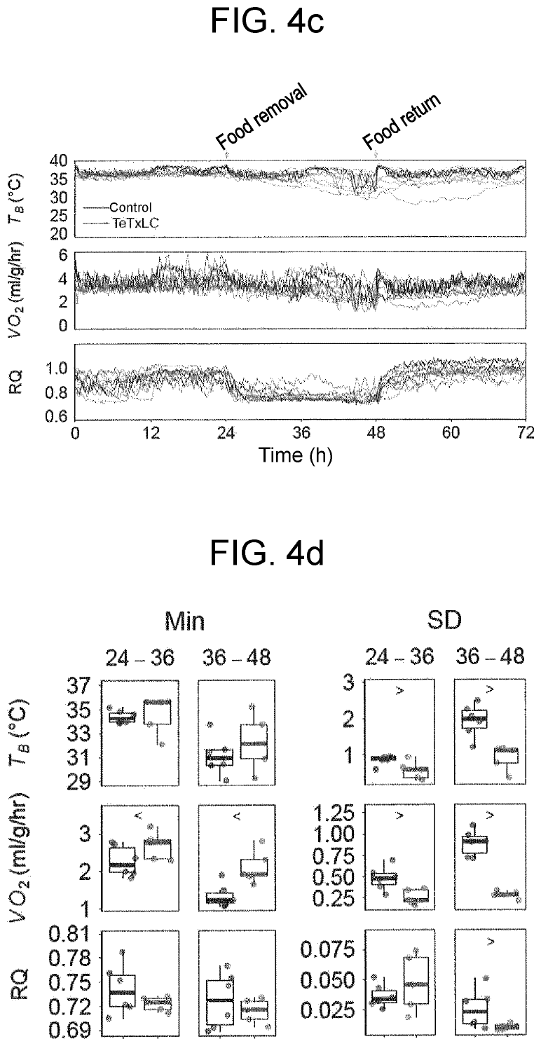

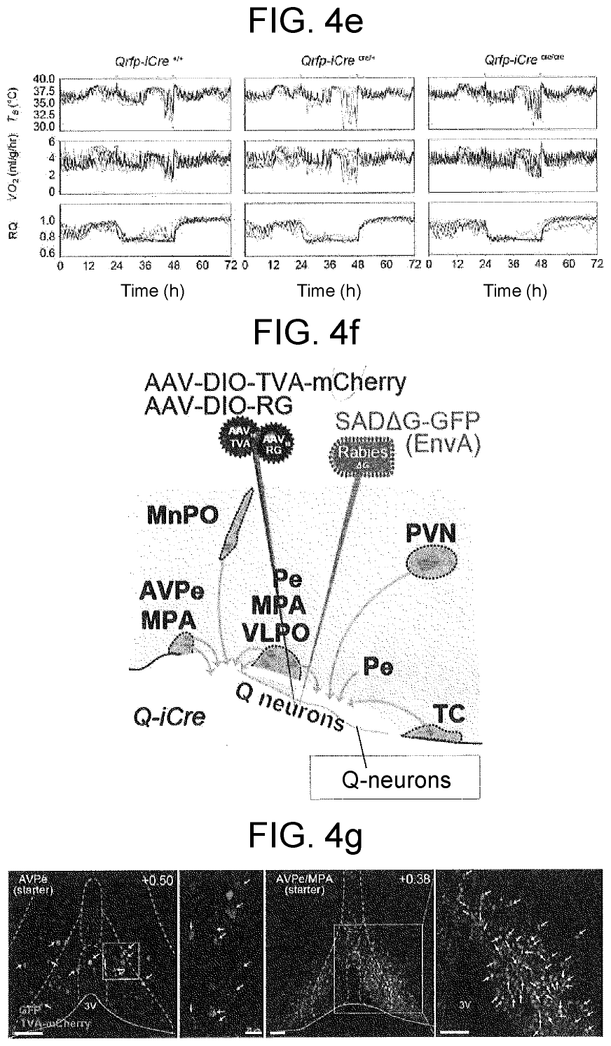

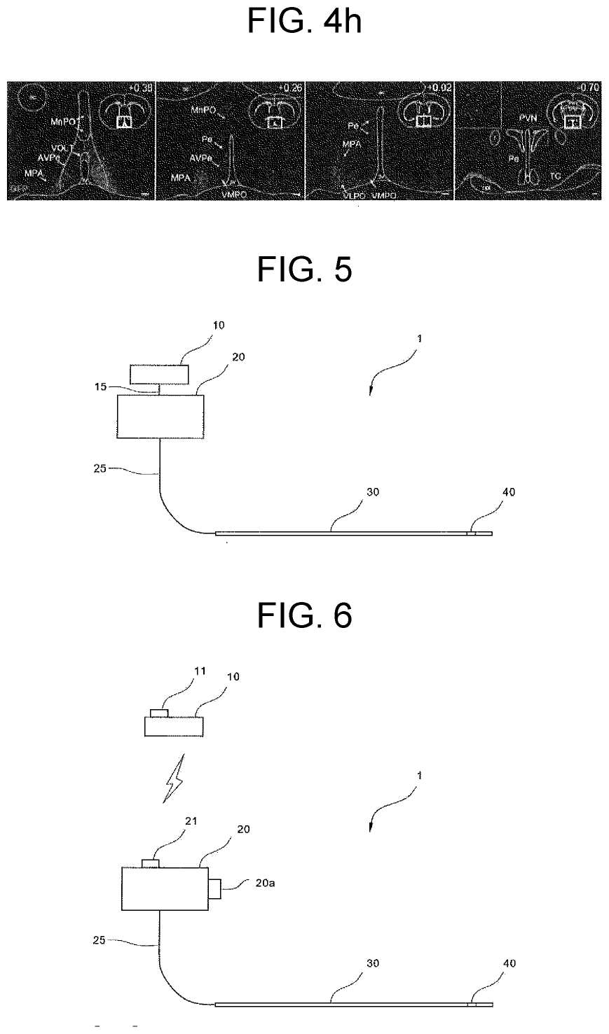

The metabolism during FIT in the Q-TeTxLC mice was modeled with a hierarchical multilayer model (FIG. 4d, code folder TeTxLC FIT). The minimum value Y of group i in section j can be expressed as the sum of the average metabolism of group β_{0[i]} and the difference parameter β_{1[i,j]}.

[Expression 6]

Y_{i,j}˜Normal(β_{0[i]}+β_{1[i,j]},σ_{ERROR}) (22)

β₀˜Normal(0,σ₀) (23)

β₁˜Normal(0,σ₁) (24)

i={1:24 to 36 h,2:36 to 48 h} (25)

j={1:Control,2: Q-TeTxLC} (26)

The identity of the mice was included as a predictor of observed values Y in order to model the dispersion of metabolism. As described above, Y of a predetermined group of a certain SECTION was modeled as a normal distribution, and this normal distribution used the mouse-dependent average α_{MOUSE} and the group and section-dependent parameter β_{GROUP}, SECTION as averages, and σ_{GROUP}, SECTION as the standard deviation.

[Expression 7]

Y_{i,j,k}˜Normal(αₖ+β_{i,j},σ_{i,j}) (27)

αₖ˜Normal(0,σ_{α}) (28)

β_{i,j}˜Normal(0,σ_{β}) (29)

σ_{i,j}˜Normal(0,σ_{σ}) (30)

i={1:Control,2: Q-TeTxLC} (31)

j={1:24-36 h,2:36-48 h} (32)

All σ of expressions (22) to (24) and (28) to (30) were sampled from standard half-normal distributions. These models were used in the modeling of T_{B}, VO₂, and RQ. Even Y of these models can theoretically accept a negative real number, and in this case, good convergence occurred, and thus this model was also applied to VO₂ and RQ.

### EXPERIMENTS AND RESULTS

### Example 1: Induction of Hypometabolism by Chemically Defined Hypothalamic Neuron Population

The pyroglutamylated RFamide peptide (QRFP), a hypothalamic neuropeptide, was originally discovered through a bioinformatics approach aimed at the discovery of a new RFamide peptide^{9,10}. The Qrfp peptide was also identified and purified from the rat brain as an endogenous ligand for the orphan G-protein-coupled receptor hGPR103¹¹. The prepro-Qrfp mRNA is localized only in the hypothalamus and is distributed in the periventricular nucleus (Pe), the lateral hypothalamus (LHA), and the tuber cinereum (TC) 11. Orfp has previously been implicated in food intake, sympathetic regulation, and anxiety^{11,12}. The inventors prepared mice (Qrfp-iCre mice) having codon-improved Cre recombinase (iCre) knocked-in into the Qrfp gene. In order to obtain mice (Qrfp-iCre; Rosa26^{dreaddm3} mice) in which hM3Dq-mCherry was expressed only in iCre-expressing neurons, the inventors crossbred CAG-hM3Dq-mCherry with Rosa26^{dreadm3} mice having an upstream floxed transcription arrest element inserted at the Rosa26 gene locus. During an excitatory chemogenetic experiment using the Qrfp-iCre; Rosa26^{dreaddm3} mice, these mice exhibited a remarkable decrease in motor activity and ultimately entered a severe and sustained immobile state that started about 30 minutes after intraperitoneal (IP) injection of clozapine-N-oxide (CNO). It was noticed that the posture of these mice was similar to the posture observed during daily torpor, and thus it was initially hypothesized that activation of iCre-positive cells in Qrfp-iCre mice induces a daily torpor-like state, which is characterized by immobility and low T_{B} (as described later, it has been revealed that the hypothermia induced here is not daily torpor, but a hibernation-like state). In order to evaluate this hypothesis, the surface body temperature (Tₛ) was measured by using a thermographic camera, and it was found that the CNO-induced immobile state of the Qrfp-iCre; Rosa26^{dreaddm3} mice was accompanied by remarkable and sustained hypothermia (FIG. 1b). The decrease in Tₛ started about 5 minutes after CNO administration and lasted for approximately 12 hours. After that, the mice spontaneously recovered from the hypothermic state without external rewarming.

In contrast, inhibitory DREADD manipulation through activation of hM4Di in iCre-positive neurons of Qrfp-iCre; Rosa26^{dreaddm4} mice did not have any effect on T_{S} (FIG. 1b). Importantly, hM3Dq-mediated activation of iCre-positive neurons in the Qrfp-iCre; Rosa26^{dreadm3} mice induced severe hypothermia, even in homozygous Qrfp-iCre mice in which the prepro-Qrfp sequence was completely replaced with iCre in both alleles (FIG. 1b). This suggests that the Qrfp peptide itself is not essential for inducing hypothermia. Rather, the degree of hypothermia is more remarkable in Qrfp knockout (Qrf-iCre homozygous) mice, suggesting the possibility that endogenous Qrfp itself counteracts hypothermia. This is consistent with the following previous observation of the inventors: Qrfp, when centrally administered, increases sympathetic nerve outflow and increases the heart rate and the blood pressure¹¹.

Therefore, Qrfp was identified as a chemical marker for hypothermia-inducing neurons. Next, iCre-positive neurons are observed only in the hypothalamus, and these are distributed in several discrete hypothalamic regions of Qrfp-iCre mice, and thus an attempt was made to identify a hypothalamic region inducing hypothermia. A Cre-activatable AAV vector having a flip-excision (FLEX) switch¹³ was injected into the hypothalamus of the Qrfp-iCre mice by using two different stereotaxic coordinates; mediobasal (MB) injection or lateral (LH) injection (see the Methods section), and thereby iCre-positive neurons in the lateral and medial regions of the hypothalamus were separately manipulated. By MB injection of the Cre-dependent AAV vector, it was possible to express specific genes of the iCre-positive neurons in the medial regions of the hypothalamus, that is, the anteroventral periventricular nucleus (AVPe), the medial preoptic area (MPA), and Pe, but it was impossible to express those in LHA (FIG. 1c). By multi-color fluorescence in situ hybridization analysis, it was confirmed that most mCherry-positive cells in these regions expressed the Qrfp mRNA. By MB injection of AAV₁₀-EF1α-DIO-hM3Dq-mCherry into the Qrfp-iCre mice, hM3Dq was expressed in this region, then an electrophysiological study using hypothalamus slices prepared from these mice was carried out, and it was confirmed that bath application of CNO strongly excited the mCherry-positive neurons. It was found that IP injection of CNO into these mice caused a deeper, longer-lasting hypothermia than the severe immobile state observed in the Qrfp-iCre; Rosa26^{dreaddm3} mice (FIG. 1b, FIG. 1d). A very low T_{B} state (below 30° C.) lasted for 48 hours or more (FIG. 1d). Immunostaining using anti-Fos and anti-mCherry antibodies revealed numerous mCherry and Fos double-positive neurons in AVPe, MPA, and Pe, confirming the in vivo excitation of these neurons by CNO (FIG. 1e).

From these observations, it was concluded that the iCre-positive neurons (these neurons will be referred later to as quiescence-inducing neurons or Q-neurons) in AVPe/MPA and Pe of the Qrfp-iCre mice are mainly responsible for the induced hypothermic state. In the following experiments, unless otherwise stated, the inventors basically used Qrfp-iCre mice that were MB injected with AAV₁₀-EF1a-DIO-hM3Dq-mCherry (referred to as Q-hM3D mice) in order to induce hypothermia.

In order to further analyze the induced hypothermic state, a telemetry temperature sensor was implanted in the abdominal cavity of each of the Q-hM3D mice, and metabolism was continuously analyzed by respiratory gas analysis (FIG. 1f). The present study confirmed that the CNO-induced hypothermic state in the Q-hM3D mice was accompanied by a remarkable decrease in O₂ consumption rate (VO₂: oxygen consumption) (FIG. 1g), and that T_{B} decreased concurrently with T_{S} after CNO administration. In contrast, excitatory DREADD manipulation of iCre-positive neurons in the lateral regions (LHA and TC) of the hypothalamus of the Qrfp-iCre mice through LH injection of AAV₁₀-EF1a-DIO-hM3Dq-mCherry did not induce hypothermia (FIG. 1g).

During the state of Q-neuron-induced hypothermia/hypometabolism (QIH), the heart rate remarkably decreased (from 758 beats/min two hours before CNO injection to 215 beats/min two hours after CNO injection) (average of n=3). The respiratory rate decreased from 333 breaths/min to an undetectable state (the tidal volume was below the detection limit). At these timings, VO₂ decreased from 3.60 to 1.17 ml/g/hr. During QIH, the mice exhibited a very-low-amplitude electroencephalogram (EEG), and this EEG clearly differed from those observed in non-rapid eye movement sleep characterized by high-amplitude slow waves. Blood serum chemical data suggests that blood glucose levels decrease during QIH, which is presumably due to a decrease in gluconeogenesis due to a decrease in sympathetic tone. These observations further suggest that many bodily functions robustly decrease along with the decrease in T_{B} and VO₂ during QIH.

A DREADD-mediated effect usually lasts for only a few hours after CNO injection, but the DREADD-induced QIH in the Q-hM3D mice lasted for a very long period of time. Surprisingly, at T_{A}=20° C., QIH with a T_{B} of less than 30° C. lasted for 48 hours or more with only one dose (1 mg/kg) of CNO, and it took about 1 week for VO₂ to fully return to normal (FIG. 1h). After recovery from QIH, the mice were healthy and seemed to behave normally. QIH was reproducible in the same mice after repeated CNO injections, indicating the reversibility of this operation (FIG. 1h).

### Example 2: Q-Neurons Act on Dorsomedial Hypothalamus to Induce QIH

Axonal projections of Q-neurons were analyzed in order to clarify the mechanism for inducing QIH. Qrfp-iCre mice were injected with AAV₁₀-EF1a-DIO-GFP to express GFP specifically in Q-neurons (FIGS. 2a, 2b), then GFP-positive fibers were observed in MPA, VOLT, the paraventricular nucleus (PVN), the supraoptic nucleus (SON), the dorsomedial hypothalamus (DMH), LHA, the tuberomammillary nucleus (TMN), the medial mammillary nucleus (MM), the periaqueductal gray (PAG), the lateral parabrachial nucleus (LPB), the locus ceruleus (LLC), the rostral ventrolateral medulla (RVLM), and the raphe pallidus nucleus (RPa) (regions involved in body temperature regulation and sympathetic nerve control) (FIG. 2c)¹⁴. The inventors found that DMH received particularly abundant projections. The positions of Q-neurons and projections to DMH were further suggested from analysis of the brain that was clarified by the ScaleS method (FIG. 2d).

Next, whether these Q-neurons are inhibitory or excitatory was confirmed by using triple-color in situ hybridization of the Q-neurons. It was confirmed that the CNO injection effectively induced QIH in Q-hM3D mice, and then these mice were subjected to in situ hybridization histochemistry. A transcription product encoding mCherry and probes encoding vesicular glutamate transporter 2 (Vglut 2) and vesicular GABA transporter (Vgat), which are excitatory and inhibitory markers, were used. The inventors also found that about two-thirds of the Q-neurons were Vgat-positive and about two-fifths were Vglut2-positive (FIGS. 2e to 2i).

Among the regions containing abundant projections by Q-neurons (FIG. 2c), the inventors focused attention on DMH. This is because heat production-promoting neurons were previously identified in DMH¹⁵. An optogenetic approach was used in order to clarify the function of axonal projections of Q-neurons to DMH. Stabilized step function opsin (SSFO) 16 was expressed in Q-neurons by injecting Qrfp-iCre mice (Q-SSFO mice) with AAV₁₀-DIO-SSFO-eYFP (FIG. 2j). The expression of SSFO in AVPe, MPA, and Pe was confirmed. In order to confirm the effect of optogenetic excitation on Tₛ, first, optical fibers were implanted in AVPe/MPA, where many cell bodies of Q-neurons are found (FIG. 2j), and the mice were subjected to photogenerated excitation of SSFO-positive cell bodies by applying a light pulse (light pulse having a 1-second width). In this state, optogenetic excitation of the Q-neurons rapidly induced strong hypothermia, which lasted for about 20 minutes (FIG. 2k). When the Q-neurons was repeatedly excited every 30 minutes a total of four times, marked hypothermia with Tₛ as low as T_{A} (22° C.) occurred. Many Fos-positive neurons were identified in SSFO-eYFP-positive cells in AVPe/MPA after excitation (FIG. 2j). Optogenetically induced QIH clearly lasted for a shorter period of time than QIH induced by hM3Dq-mediated pharmacogenetic excitation of Q-neurons (FIG. 2k), and this suggests the possibility that Gq-mediated metabotropic signaling in Q-neurons leading to a change in gene expression profile serves to create the long-lasting nature of QIH.

Next, optical fibers were implanted in DMH bilaterally in the Q-SSFO mice, and light stimulation was applied to the axonal fibers. This manipulation effectively reduced Tₛ, but this reduction was slightly weaker than that induced by somatic stimulation in AVPe/MPA (FIGS. 2k, 2l). It is known that RPa includes sympathetic premotor neurons for heat production via brown adipose tissue control¹⁷, and thus as a control, the influence of light stimulation on Q-neuron fibers in RPa was also examined. In addition, subtle actions of optogenetic excitation of the Q-neuron fibers in RPa on Tₛ were also observed (FIGS. 2k, 2l). From these results, it was hypothesized that Q-neurons act mainly on DMH and act to a lesser extent on RPa to induce QIH.

### Example 3: Theoretical Setpoint Temperature Decreases During QIH

An increase in the temperature of the tail of the mice was observed immediately after QIH induction, and QIH was induced by optogenetic or pharmacogenetic excitation of Q-neurons, thus suggesting that peripheral vessels dilate to release heat during T_{B} reduction (FIG. 1d, FIG. 2k). Peripheral vasodilatation without an increase in T_{B} suggests that the theoretical body temperature setpoint (T_{R}) was reset to a value lower than in the normal state, as seen in the hibernating state of hibernating animals. In order to evaluate this, feature analysis of the thermoregulatory system of mice during QIH was carried out. Heat conductance (G), H, and T_{R} can be estimated from T_{B} and VO₂ at a plurality of ambient temperatures (T_{A}) under conditions in which an animal is not engaged in external work and has a stable metabolism³. Q-hM3D mice were prepared, and T_{B} and VO₂ were recorded during QIH at various T_{A}s (8, 12, 16, 20, 24, 28, and 32° C.) (FIG. 3a). The average T_{B} and VO₂ eleven hours after IP injection of physiological saline or CNO were compared. During QIH, the animals were compared with a corresponding control, and exhibited lower T_{B} and VO₂ at every T_{A} (FIG. 3b). When the heat production system is functioning properly, that is, when T_{R} is higher than T_{B} and the thermoregulatory system increases VO₂ in an attempt to reach T_{R} (FIG. 3c), T_{B} increases and VO₂ decreases as T_{A} increases. T_{B} and VO₂ exhibited minimum values at different T_{A} from each other, and a coordinated heat generation property was exhibited only in a T_{A} range of 16 to 24° C. (FIG. 3b). Therefore, further analysis was carried out by using metabolism data at T_{A}=16, 20, and 24° C. during QIH. First, G was estimated from the relationship of T_{B}−T_{A} and VO₂ (FIG. 3d). Under normal and QIH conditions, the 89% highest posterior density interval (HPDI) of G was [0.212, 0.221] ml/g/hr/° C. and [0.182, 0.220] ml/g/hr/° ° C., respectively (FIG. 3e; hereafter, the 89% HPDI is indicated by two numbers in square brackets). Quantitatively, the posterior distribution of the difference between both G (AG) is [−0.0040, 0.0348] ml/g/hr/° C. (FIG. 3f), which includes 0, suggesting that G under normal conditions and G under QIH conditions are indistinguishable. This differed from daily torpor, during which a lower G was exhibited than under normal conditions³. Second, H and T_{R} were estimated from T_{B} and VO₂ (FIG. 3g). H was [3.43, 8.72] ml/g/hr/° C. in the normal state and was [0.181, 0.369] ml/g/hr/° C. in QIH (FIG. 3h), indicating a 95.3% reduction in the median in QIH from that in the normal state. The posterior distribution of the difference (ΔH) was [3.17, 8.48] ml/g/hr/° C. (FIG. 3i), which was positive, suggesting that the probability that these conditions differ is 89% or more. This decrease in H was similar to the decrease in H during fasting-induced daily torpor (FIT) 3. In particular, T_{R} was estimated to be [36.04, 36.60]° C. in the normal state, and [26.83, 29.13]° C. in QIH (FIG. 3j). The difference between the medians of T_{R} was 8.41° C., and the posterior distribution of the difference (ΔT_{R}) was [7.18, 9.57]° C., clearly indicating a decrease in T_{R} during QIH (FIG. 3k). In consideration of the very small shift³ in theoretical setpoint temperature in FIT, this observation emphasizes the similarity between QIH and hibernation and the difference between QIH and daily torpor.

In order to provide further evidence of the reduction in T_{R} during QIH, which is a remarkable feature of hibernation, the relationship between the postures and the metabolism of each mouse was observed when T_{A} was dynamically changed during QIH (n=4, representative data for one mouse in FIG. 3l, and data for other three mice). The very stable, long-term hypometabolic state in QIH as in a hibernating animal made it possible to investigate this in mice. A Q-hM3D mouse was set at T_{A}=28° C., and FIT was induced (A and B in FIG. 3l). After a recovery period of 24 hours, QIH was induced by administering CNO (C, D, E, and F in FIG. 3l).

Interestingly, at T_{A}=28° C., the mouse exhibited an extended posture during QIH, and this posture is normally seen in animals exposed to a high temperature environment (D in FIG. 3l). This was clearly different from the typical sitting posture that was observed during FIT at T_{A}=28° C. (B in FIG. 3l). This behavior observation further shows that T_{R} is lower in QIH than in FIT and in the normal state. Further, when T_{A} was lowered to 12° C., the mouse returned to a sitting posture (E in FIG. 3l) similar to that in daily torpor, and started shivering. These results strongly support the hypothesis that during QIH, T_{R} decreases, but bodily functions and behavior are still regulated to adapt to a change in T_{A}.

It is well known that during hibernation, the metabolic rate of animals is low and that the metabolism is actively regulated in response to T_{A}. Similarly, in QIH, animals exposed to a T_{A} of 16° C. or less exhibited a considerably larger VO₂ than animals exposed to a T_{A} of 20° C. or 28° C. (FIG. 3b). In fact, this is similar to a previous report¹⁸ relating to hibernating animals that exhibited an increase in metabolism when T_{A} is lowered to a certain level. This feature of active hypometabolism in QIH was also confirmed in individual animals (FIG. 3l). The behavior and metabolic reactions of mice during QIH were completely different than those observed in the normal state in which the bodily functions attempted to maintain T_{B} in a narrow range.

In order to compare metabolic functions during QIH with those in an anesthetized state, the inventors recorded the metabolic transition during general anesthesia at a plurality of T_{A}. As expected, animals under anesthesia did not exhibit an increase in VO₂ or a change in posture even when exposed to a low T_{A}. In addition, the metabolic state induced by systemic delivery of the adenosine A1AR agonist (6)N-cyclohexyladenosine (CHA), which is used to induce a hypothermic state, was also investigated¹⁹. IP injection of CHA (2.5 mg/kg) into wild-type mice effectively induced a hypothermic/hypometabolic state, but the mice did not react to a low T_{A} (12° C.) through either an increase in VO₂ or behavior (posture and shivering). T_{B} at 20° ° C. tended to be higher in CHA-induced hypothermia than in QIH, but T_{B} and VO₂ further decreased in CHA-induced hypothermia. On the other hand, when T_{A} was set to 12° C., these parameters increased in QIH (FIG. 3b). These observations indicate that QIH is completely different from general anesthesia or CHA-induced passive hypothermia and that a hypothermic state is induced by blocking the T_{B} regulatory system.

### Example 4: Q-Neurons are Involved in Normal Fasting-Induced Daily Torpor

QIH is more similar to hibernation than daily torpor, but daily torpor may be thought to be a mild state of hibernation, and thus whether or not Q-neurons are also involved in daily torpor was examined. In addition, a common or similar mechanism may serve to induce hibernation and daily torpor²⁰. In order to investigate the role of Q-neurons in daily torpor, Qrfp-iCre mice (Q-TeTxLC mice) were injected with AAV_{2/9}-hSyn-DIO-TeTxLC-eYFP to express a tetanus toxin light chain (TeTxLC) specifically in Q-neurons, and whether or not blocking of SNARE complex-mediated neurotransmission in the Q-neurons affects FIT was investigated (FIG. 4b). Simultaneous injection of AAV_{2/9}-hSyn-DIO-TeTxLC-eYFP and AAV₁₀-EF1α-DIO-hM3Dq-mCherry completely eliminated the action of CNO on T_{B}, suggesting that blocking of the SNARE complex eliminates the QIH-inducing capacity of Q-neurons. The inventors found that the normal structure of FIT was disrupted in all of the Q-TeTxLC mice. No rapid oscillatory fluctuations in metabolism were observed in these mice during fasting (FIGS. 4c, 4d). This suggests that the function of Q-neurons is necessary for inducing a rapid decrease in T_{B} during FIT. Interestingly, the gradual decrease in T_{B} observed in these mice means that a Q-neuron-independent metabolism-decreasing mechanism is present during FIT. In addition, the Q-TeTxLC mice had fewer circadian fluctuations in T_{B} than the control mice, suggesting that Q-neurons have a major role in the circadian regulation of T_{B}. In particular, homozygous Qrfp-iCre mice lacking the QRFP peptide exhibited normal FIT (FIG. 4e). These observations suggest that Q-neurons are an essential constituent element for inducing daily torpor and play an important role in the rapid shift in body temperature in daily torpor, but QRFP does not.

In order to clarify a neuronal mechanism for regulating the activity of Q-neurons, the inventors identified an upstream neuronal population coming into direct synaptic contact with Q-neurons by recombinant pseudotyped rabies virus vector (SADΔG(EnvA))-mediated labeling²¹ (FIG. 4f). TVA-mCherry and rabies glycoprotein (RG) were expressed in Q-neurons by using Cre-activatable AAV vectors²² in Qrfp-iCre mice, and then SADΔG-GFP (EnvA) was injected into the same site. Starter cells that were double-positive for TVA-mCherry and GFP were found in AVPe/MPA and Pe (FIG. 4g). Input neurons providing a direct synaptic input to Q-neurons (GFP-positive but mCherry-negative) were identified in the median preoptic nucleus (MnPO), PVN, and MPA (FIG. 4h). The input neurons were also observed inside and around AVPe/Pe, suggesting the presence of local interneurons regulating the function of Q-neurons, and the possibility that Q-neurons constitute microcircuits with interneurons within AVPe/MPA and Pe. These observations suggest that Q-neurons receive relatively sparse direct inputs from intrahypothalmic regions. FIT is induced by fasting, and thus Q-neurons are expected to monitor negative energy balance. Neurons in PVH have been shown to receive abundant input from ARC²³, and thus the input from PVH to Q-neurons may serve to convey information about the nutritional state. The PVH input may also convey circadian information from the suprachiasmatic nucleus (SCN).

MPA is involved in the regulation of T_{B}^{24,25}, and thus reciprocal interaction between Q-neurons and MPA may play an important role in body temperature regulation. Input neurons are also included in VMPO. In a previous study, warmth-sensitive neurons in ventromedial preoptic nucleus (VMPO) were identified as BDNF and PACAP double-positive neurons. Excitation of these cells also induced hypothermia²⁶. This effect is much smaller than that induced by the excitation of Q-neurons, and functional interaction may be present between VMPOPACAP/BDNF neurons and Q-neurons. In addition, DREADD excitation of TRPM2-positive cells in POA was shown to induce hypothermia. TRPM2 is highly expressed ubiquitously in AVPe/MPA and POA including a region containing input neurons to Q-neurons, and thus TRPM2-induced hypothermia may be induced by direct and/or indirect activation of Q-neurons²⁷.

Q-neurons are localized along the third ventricle (3V), and dendrites of these neurons extend along the ependyma of 3V and regions near circumventricular organs (FIG. 1c), and thus the Q-neurons may also sense humoral factors that are released by tanycytes and ependymal cells, factors in the cerebrospinal fluid, or capillary vessels.

### [Discussion]

Here, the presence of a novel hypothalamic neuron population having specific chemical (=expressing Qrfp) and tissue-based (AVPe/MPA) characteristics in mice is shown, and excitation of this population induces an active hypometabolism that is very similar to hibernation. QIH, which is this state, shares two major properties with hibernation. The first is a decrease in T_{R}, and the second is actively regulated hypometabolism. During QIH, mice actively regulate bodily functions thereof according to the external environment. Many other physiological parameters such as a decrease in heart rate, weak respiration, and a low-voltage electroencephalogram, suggest a similarity between QIH and hibernation²⁹. Through analysis of Fos expression, a previous study showed that cells near 3V are activated during hibernation in thirteen-lined ground squirrels³⁰. This activation pattern is very similar to the region in which Q-neurons are localized, suggesting the possibility that hibernation nerves also use Q-neurons to induce hibernation.

It is very surprising that the mice were able to enter a hibernation-like state (=QIH). Distantly related mammals including rodents, the California, and even primates have the ability to hibernate, and thus the neuronal mechanism of hibernation is conserved among a wide range of mammalian species, and it is reasonable to hypothesize that these systems are not mobilized in non-hibernating animals in the normal state. The Qrfp gene is conserved even in humans, and thus it can also be inferred that an active hypometabolic state may be exhibited when Q-neurons are excited. In the present study, it was also confirmed that DMH is a major effector site for Q-neurons. Future studies identifying QIH-induced neurons in DMH will further clarify the mechanism of QIH. Q-neurons may also act on other regions identified in the present study. For example, it was recently reported that SON plays an important role in general anesthesia and sleep³².

In addition, it was found that Q-neurons are necessary for fasting-induced daily torpor in mice. However, the detection of an increase in Q-neuron activity during fasting of mice through Fos staining or fiber photometry (data not shown) was unable to be repeated, suggesting the possibility that the low level activation of Q-neurons is sufficient for inducing hypothermia in daily torpor.

The induced hibernation in non-hibernating animals shown in the present study is a promising advance toward understanding the neuronal mechanism of active hypometabolism, and the present study provides a method for examining how each tissue adopts a hibernation-like hypometabolic state. Further, with the future development of a method for selectively exciting Q-neurons, QIH could provide a new approach for the development of a method that enables clinical application of synthetic hibernation in humans with great benefits in medicine that may reduce systemic tissue damage after heart attack or stroke or is useful in organ transplant preservation.

When the experiments described above were carried out in rats instead of mice, a QIH state was able to be induced⁴³. Specifically, AAV₁₀-CaMKIIα-hM3Dq-mCherry was injected into AVPe/MPA in the rat brain. Addition of CNO activated left and right AVPe/MPA including Q-neurons and induced QIH with hypothermia and hypometabolism in rats. In the rats having the QIH, an extended posture was observed with a decrease in VO₂. These were similar to those in mice in the QIH state. Selective activation of Q-neurons also induced QIH in mammalian animals that do not normally hibernate, such as rats.

### Example 5: Treatment of Acute Disease by Hibernation

In the present invention, the effect of treating a subject having an acute disease by hibernation was confirmed. As the acute disease, sepsis and acute renal failure were tested.

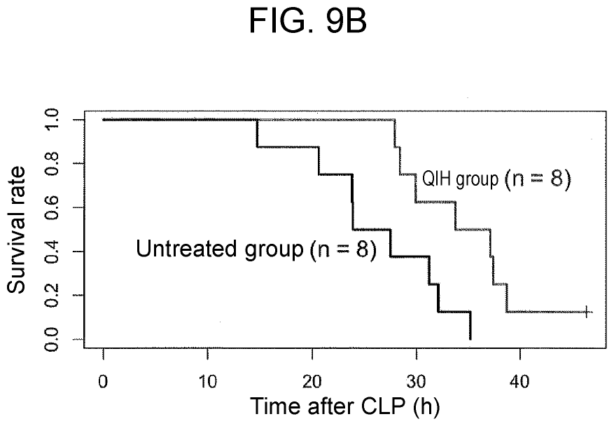

Sepsis was induced in a cecal ligation and puncture (CLP) model (see FIG. 9A).

Mice were anesthetized with isoflurane. A longitudinal midline incision was made in the skin with a scalpel while taking care not to enter the peritoneal cavity. After the initial incision, the incised part was widened with small scissors. The cecum was removed and ligated in a predetermined place. Before cecal perforation, the contents of the cecum were gently pushed toward the distal cecum, and during cecal perforation, trapped air or gas was gently aspirated. The cecum was perforated with a single through-puncture in a mesenteric to anti-mesenteric direction at the midpoint between the ligated part and the tip of the cecum. After withdrawal of the needle, a small amount of excreta (droplets) was extruded from both the mesenteric through-hole and the anti-mesenteric through-hole to confirm patency. Feces from the cecum were redisposed into the abdominal cavity without spreading over the peritoneal wall wound edge. The peritoneum, the fascia, and the abdominal fascia were closed with simple running sutures. The surgery was performed under proper temperature control.

After CLP, hibernation (QIH) was induced in the mice. Hibernation was not induced in the negative control. The survival rates of the mice were observed for 48 hours after CLP (see FIG. 9A). Results thereof are as shown in FIG. 9B. As shown in FIG. 9B, the group in which hibernation was induced (QIH group) showed prolonged survival time compared with the negative control.

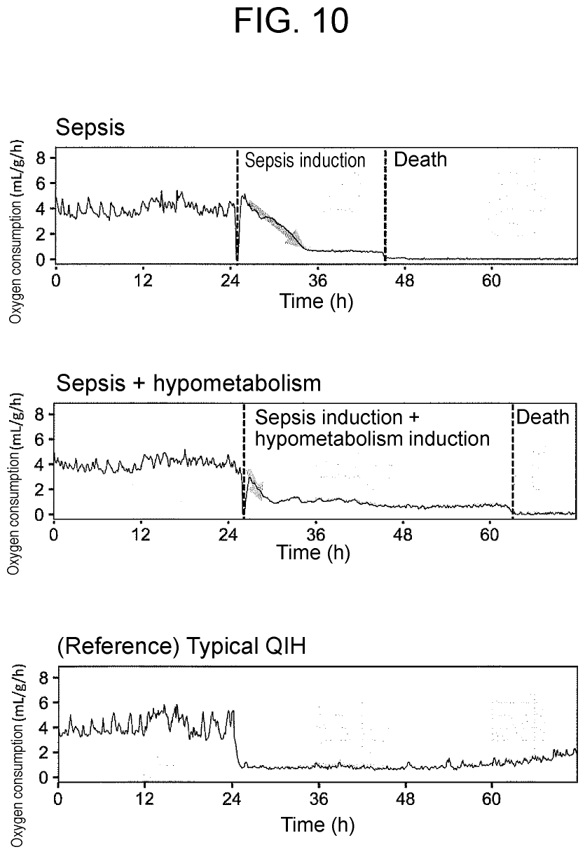

FIG. 10 shows changes in oxygen consumption over time in the sepsis model, changes in oxygen consumption over time in the model in which hibernation was induced immediately after induction of sepsis, and changes in oxygen consumption over time in typical QIH. In the sepsis model, the oxygen consumption decreases over about 10 hours, whereas in hibernation induction, the oxygen consumption decreases in about 1 to 2 hours. Hypometabolism induced in sepsis leads to decreased oxygen consumption in about 1 to 2 hours, supporting this being QIH.

Acute renal failure was induced in a renal pedicle clamp model.

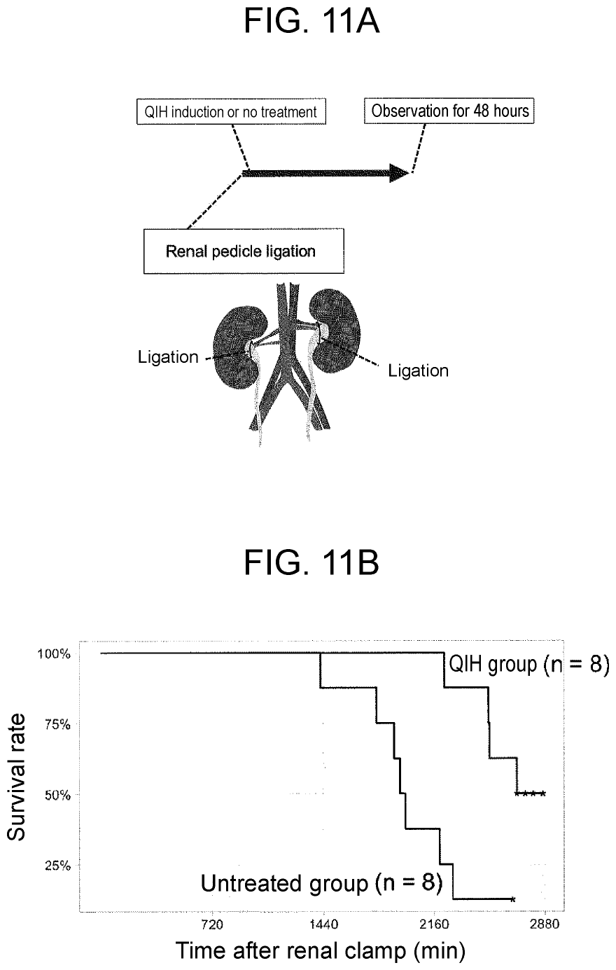

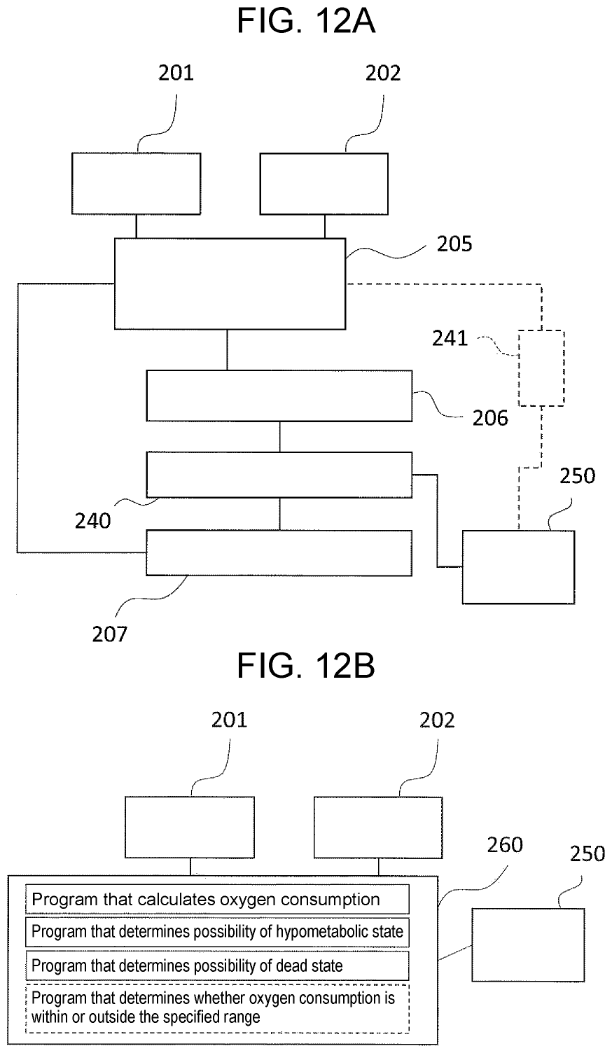

As shown in FIG. 11A, mice were subjected to general anesthesia with inhalation anesthesia. A skin incision and an abdominal wall muscle incision were made to expose the kidney. The adipose tissue surrounding the kidney was stripped to expose the renal pedicle, and the renal pedicle was ligated. After that, the wound was closed. After renal pedicle ligation, hibernation (QIH) was induced in the mice. Hibernation was not induced in the negative control. The survival rates of mice were observed over 48 hours from CLP. Results thereof are as shown in FIG. 11B. As shown in FIG. 11B, the group in which hibernation was induced (QIH group) showed prolonged survival time compared with the negative control.

### REFERENCES

1. Bouma, H. R. et al. Induction of torpor: Mimicking natural metabolic suppression for biomedical applications. J. Cell. Physiol. 227, 12851290 (2012).
2. Geiser, F. Metabolic Rate and Body Temperature Reduction During Hibernation and Daily Torpor. Annu. Rev. Physiol. 66, 239274 (2004).
3. Sunagawa, G. A. & Takahashi, M. Hypometabolism during Daily Torpor in Mice is Dominated by Reduction in the Sensitivity of the Thermoregulatory System. Sci. Rep. 6, 37011 (2016).
4. Florant, G. L. & Heller, H. C. CNS regulation of body temperature in euthermic and hibernating marmots (Marmota flaviventris). Am. J. Physiol. 232, R203-8 (1977).
5. Heller, H. C. & Colliver, G. W. CNS regulation of body temperature during hibernation. Am. J. Physiol. 227, 5839 (1974).
6. Iliff, B. W. & Swoap, S. J. Central adenosine receptor signaling is necessary for daily torpor in mice. AJP Regul. Integr. Comp. Physiol. 303, R477R484 (2012).
7. Melvin, R. G. & Andrews, M. T. Torpor induction in mammals: recent discoveries fueling new ideas. Trends Endocrinol. Metab. 20, 490498 (2009).
8. Griko, Y. & Regan, M. D. Synthetic torpor: A method for safely and practically transporting experimental animals aboard spaceflight missions to deep space. Life Sci. Sp. Res. 16, 101107 (2018).
9. Fukusumi, S. et al. A New Peptidic Ligand and Its Receptor Regulating Adrenal Function in Rats. J. Biol. Chem. 278, 4638746395 (2003).
10. Chartrel, N. et al. Identification of 26RFa, a hypothalamic neuropeptide of the RFamide peptide family with orexigenic activity. Proc. Natl. Acad. Sci. 100, 1524715252 (2003).
11. Takayasu, S. et al. A neuropeptide ligand of the G protein-coupled receptor GPR103 regulates feeding, behavioral arousal, and blood pressure in mice. Proc Natl Acad Sci USA 103, 74387443 (2006).
12. Okamoto, K. et al. QRFP-Deficient Mice Are Hypophagic, Lean, Hypoactive and Exhibit Increased Anxiety-Like Behavior. PLOS One 11, e0164716 (2016).
13. Atasoy, D., Aponte, Y., Su, H. H. & Sternson, S. M. A FLEX switch targets Channelrhodopsin-2 to multiple cell types for imaging and long-range circuit mapping. J. Neurosci. 28, 702530 (2008).
14. Nakamura, K. Central circuitries for body temperature regulation and fever. Am. J. Physiol. Integr. Comp. Physiol. 301, R1207R1228 (2011).
15. Zhao, Z.-D. et al. A hypothalamic circuit that controls body temperature. Proc. Natl. Acad. Sci. U.S.A. 114, 20422047 (2017).
16. Yizhar, O. et al. Neocortical excitation/inhibition balance in information processing and social dysfunction. Nature 477, 171 (2011).
17. Morrison, S. F. Central control of body temperature. F1000Research 5, (2016).
18. Ortmann, S. & Heldmaier, G. Regulation of body temperature and energy requirements of hibernating alpine marmots (Marmota marmota). Am. J. Physiol. Regul. Integr. Comp. Physiol. 278, R698-704 (2000).
19. Tupone, D., Madden, C. J. & Morrison, S. F. Central activation of the A1 adenosine receptor (A1AR) induces a hypothermic, torpor-like state in the rat. J. Neurosci. 33, 1451225 (2013).
20. Heldmaier, G., Ortmann, S. & Elvert, R. Natural hypometabolism during hibernation and daily torpor in mammals. Respir. Physiol. Neurobiol. 141, 317329 (2004).
21. Wickersham, I. R. et al. Monosynaptic Restriction of Transsynaptic Tracing from Single, Genetically Targeted Neurons. Neuron 53, 639647 (2007).
22. Saito, Y. C. et al. Monoamines Inhibit GABAergic Neurons in Ventrolateral Preoptic Area That Make Direct Synaptic Connections to Hypothalamic Arousal Neurons. J. Neurosci. 38, 63666378 (2018).
23. Andermann, M. L. & Lowell, B. B. Toward a Wiring Diagram Understanding of Ap petite Control. Neuron 95, 757778 (2017).
24. Wang, T. A. et al. Thermoregulation via Temperature-Dependent PGD2 Production in Mouse Preoptic Area. Neuron 114 (2019). doi:10.1016/j.neuron.2019.04.035
25. Tan, C. L. & Knight, Z. A. Review Regulation of Body Temperature by the Nervous System. Neuron 98, 3148 (2018).
26. Tan, C. L. et al. Warm-Sensitive Neurons that Control Body Temperature. Cell 167, (2016).
27. Song, K. et al. The TRPM2 channel is a hypothalamic heat sensor that limits fever and can drive hypothermia. Science (80-.). 353, 13931398 (2016).
28. Oomura, Y. et al. A new brain glucosensor and its physiological significance. Am. J. Clin. Nutr. 55, 278S-282S (1992).
29. Walker, J. M., Glotzbach, S. F., Berger, R. J. & Heller, H. C. Sleep and hibernation in ground squirrels (Citellus spp): electrophysiological observations. Am. J. Physiol. Integr. Comp. Physiol. 233, R213R221 (1977).
30. Bratincsak, A. et al. Spatial and temporal activation of brain regions in hibernation: c-fos expression during the hibernation bout in thirteen-lined ground squirrel. J. Comp. Neurol. 505, 44358 (2007).
31. Dausmann, K. H., Glos, J., Ganzhorn, J. U. & Heldmaier, G. Hibernation in a tropical primate. Nature 429, 825826 (2004).
32. Jiang-Xie, L.-F. et al. A Common Neuroendocrine Substrate for Diverse General Anesthetics and Sleep. Neuron 113 (2019). doi:10.1016/j.neuron.2019.03.033
33. Mieda, M. et al. Cellular clocks in AVP neurons of the scn are critical for interneuronal coupling regulating circadian behavior rhythm. Neuron 85, 11031116 (2015).
34. Osakada, F. & Callaway, E. M. Design and generation of recombinant rabies virus vectors. Nat. Protoc. 8, 1583601 (2013).
35. Sunagawa, G. A. et al. Mammalian Reverse Genetics without Crossing Reveals Nr3a as a Short-Sleeper Gene. Cell Rep. 14, 662677 (2016).
36. Keith, B. J., Franklin & Paxinos, G. The mouse brain in stereotaxic coordinates. (Academic Press, 2007).
37. Hama, H. et al. ScaleS: an optical clearing palette for biological imaging. Nat. Neurosci. 18, 15181529 (2015).
38. Stan Development Team. RStan: the R interface to Stan. (2018).
39. R Core Team. R: A language and environment for statistical computing. (2017).
40. McElreath, R. Statistical Rethinking: A Bayesian Course with Examples in R and Stan. (CRC Press, 2016).
41. Gunn, A. J., Laptook, A. R., Robertson, N. J., Barks, J. D., Thoresen, M., Wassink, G., and Bennet, L. (2017). Therapeutic hypothermia translates from ancient history in to practice. Pediatr. Res. 81, 202-209.
42. Nielsen, N., Wetterslev, J., Cronberg, T., Erlinge, D., Gasche, Y., Hassager, C., Horn, J., Hovdenes, J., Kjaergaard, J., Kuiper, M., et al. (2013). Targeted temperature management at 33 C versus 36 C after cardiac arrest. N. Engl. J. Med. 369, 2197-2206.
43. Takahashi, T. et al. (2020). A discrete neuronal circuit induces a hibernation-like state in rodents. Nature, 583, 109-114.

1. An apparatus that monitors a hypometabolic state in a subject having a disease in a controlled hypometabolic state, the apparatus comprising:
a measurement unit that measures an oxygen concentration of each exhaled air and inhaled air from the subject;
a computation unit that calculates an oxygen consumption of the subject from an oxygen concentration difference between the exhaled air and the inhaled air from the subject; and
a hypometabolic state monitoring unit that monitors the oxygen consumption over time, and estimates that the subject is in a hypometabolic state when the oxygen consumption decreases compared with before the hypometabolic state, and estimates that the subject is dead or may be dead when the oxygen consumption is zero.

2. The apparatus according to claim 1, wherein the controlled hypometabolic state is a hibernation-like state.

3. The apparatus according to claim 1, wherein the apparatus further comprises a determination unit that records a fluctuation in oxygen consumption for a certain period of time in the subject in a stable hypometabolism state, sets a specified range based on the fluctuation in oxygen consumption for a certain period of time, and determines whether the oxygen consumption is within or outside the specified range.

4. The apparatus according to claim 1, wherein the apparatus further comprises an output device, and the apparatus, when the oxygen consumption exceeds an upper limit of the specified range, outputs to the output device an indication that the subject may be waking up from the controlled hypometabolic state; when the oxygen consumption decreases below a lower limit of the specified range, outputs to the output device an indication that the subject may be transitioning from the controlled hypometabolic state to greater hypometabolism; and when the oxygen consumption is within the specified range, outputs to the output device an indication that the subject is in the controlled hypometabolism state.

5. The apparatus according to claim 1, wherein the apparatus further comprises a deep body thermometer, a body temperature storage unit that stores a body temperature measured by the thermometer, a computation unit that calculates a theoretical setpoint temperature, and a computation unit that calculates a feedback gain (H) of heat generation.

6. An apparatus that stimulates a pyroglutamylated RFamide peptide (QRFP)-producing neuron within one or more regions selected from the group consisting of an anteroventral periventricular nucleus (AVPe), a medial preoptic area (MPA), and a periventricular nucleus (Pe) in a brain of a living subject, wherein the subject has a disease, and
the apparatus comprises:
a control unit that transmits a control signal controlling voltage generation;
a voltage generation unit that receives the control signal from the control unit and generates a voltage;
a stimulation probe electrically connected proximally to the voltage generation unit and comprising an electrical stimulation electrode distally wherein the stimulation probe has a length sufficient to access the QRFP-producing neuron from a brain surface, and generates an electrical stimulus at a distal electrical stimulation electrode with the voltage from the voltage generation unit;
an outside air temperature gauge;
a deep body thermometer;
a gas analysis unit that measures an oxygen concentration of each of exhaled gas and inhaled gas;
a recording unit that records a measured outside air temperature and at least one numerical value selected from the group consisting of a deep body temperature and the oxygen concentration;
a computation unit that calculates an oxygen consumption of the subject from an oxygen concentration difference between exhaled air and inhaled air from the subject;
a hibernation possibility estimation unit that estimates that the subject has entered a hibernation-like state or may have entered a hibernation-like state at least based on a decrease in both oxygen consumption and theoretical setpoint temperature compared with an oxygen consumption and a theoretical setpoint temperature of the subject in a non-hibernation-like state; and
a death possibility estimation unit that estimates that the subject is dead or may be dead at least when the oxygen consumption is zero.

7. (canceled)

8. The apparatus according to claim 6, wherein the apparatus further comprises a determination unit that determines whether the subject is in a hypothermic state from the outside air temperature and the deep body temperature recorded in the recording unit.

9. The apparatus according to claim 6, wherein the apparatus further comprises a determination unit that determines whether or not the subject is in a hypometabolic state from the outside air temperature, the deep body temperature, and the oxygen concentration recorded in the recording unit.

10. The apparatus according to claim 6, wherein a hibernation possibility determination unit further comprises a determination unit that determines whether or not the subject is in a hibernation-like state based on the outside air temperature, the deep body temperature, and the oxygen concentration recorded in the recording unit.

11. The apparatus according to claim 8, wherein the control unit transmits a control signal for stimulating the GRFP-producing neuron continuously or intermittently until it is determined that the subject is in any one state selected from the group consisting of a hypothermic state, a hypometabolic state, and a hibernation-like state.

12.-17. (canceled)

18. A method for treating a mammalian animal having a disease, the method comprising:
inducing a controlled hypometabolic state in the animal.

19. The method according to claim 18, wherein
the inducing a controlled hypometabolic state in the mammalian animal is carried out in such a way as to slow progression of the disease in the mammalian animal.

20. The method according to claim 18, wherein
the inducing a controlled hypometabolic state in the mammalian animal is carried out in such a way as to decrease mortality of the mammalian animal.

21. The method according to claim 18, wherein the controlled hypometabolic state is a hibernation-like state.

22. The method according to claim 18, wherein the inducing a controlled hypometabolic state is carried out by stimulating a QRFP neuron.

23. The method according to claim 18, wherein the disease is a serious acute disease.

24. The method according to claim 18, wherein the method further comprises following up the animal.

25. The method according to claim 24, wherein the following up comprises measuring a change in oxygen consumption of the animal over time.

26.-28. (canceled)
